(19) United States
(12) Patent Application Publication
(10) Pub. No.: US 2026/0117215 A1
(43) Pub. Date: Apr. 30, 2026

(54) PROTEOLYTIC ENZYMES

(71) Applicant: DIGESTIVA, INC., Davis, CA (US)

(72) Inventors: Kevin E. MCBRIDE, Davis, CA (US); Wai Shun MAK, Davis, CA (US)

(21) Appl. No.: 19/418,612

(22) Filed: Dec. 12, 2025

Related U.S. Application Data
(60) Provisional application No. 63/683,600, filed on Aug. 15, 2024.

(51) Int. Cl. C12N 9/52

(52) U.S. Cl. CPC C12N 9/52; C12Y 304/211

(57) ABSTRACT

The present disclosure provides proteolytic enzymes, i.e., S53 family proteases, having increased thermostability, increased enzymatic activity, and/or increased substrate specificity and which can digest a variety of food proteins, thereby enhancing a food's protein bioavailability. The S53 family proteases have amino acid sequences that are at least 80% identical to SEQ ID NO: 1 and comprises at least one mutation in the amino acid sequence's 513-loop which corresponds to amino acids 513 to 517 of SEQ ID NO: 1, wherein the at least one mutation is located at position 513, 514, 515, and/or 517.

### CROSS-REFERENCE TO RELATED APPLICATIONS

This application claims priority to U.S. Provisional Application No. 63/683,600, filed 15 Aug. 2024, the entire contents of which are incorporated herein by reference.

### SEQUENCE LISTING

The instant application contains a Sequence Listing which has been submitted electronically in XML format and is hereby incorporated by reference in its entirety. Said XML copy, created on 8 Jan. 2026, is named “3000203-001001_Sequence_Listing.xml” and is 12,368 bytes in size.

### BACKGROUND

The nutritional value of a food protein lies, in part, in its bioavailability following proteolytic digestion in one's digestive systems. Not all food proteins are sufficiently digested and/or resist proteolytic digesting in the gut, thereby limiting the food protein's nutritional value and bioavailability. Thus, the full nutritional value of the food protein cannot be achieved. The present disclosure addresses this and other needs.

### SUMMARY

The present disclosure provides proteolytic enzymes, i.e., proteases, having increased thermostability, increased enzymatic activity, and/or increased substrate specificity and which can digest a variety of food proteins, thereby enhancing a food's protein bioavailability.

An aspect of the present disclosure provides an S53 family protease having an amino acid sequence that is at least 80% identical to SEQ ID NO: 1 and includes at least one mutation in the amino acid sequence's 513-loop which corresponds to amino acids 513 to 517 of SEQ ID NO: 1, wherein the at least one mutation is located at position 513, 514, 515, and/or 517.

Another aspect of the present disclosure provides an S53 family protease having an amino acid sequence that is identical to SEQ ID NO: 1 except it includes at least one mutation in the amino acid sequence's 513-loop which corresponds to amino acids 513 to 517 of SEQ ID NO: 1, wherein the at least one mutation is located at position 513, 514, 515, and/or 517.

A further aspect of the present disclosure provides an S53 family protease having an amino acid sequence that is at least 80% identical to SEQ ID NO: 1 and includes at least one mutation in the amino acid sequence's 513-loop which corresponds to amino acids 513 to 517 of SEQ ID NO: 1, wherein the at least one mutation is located at position 513, 514, 515, and/or 517, wherein the hydrophobicity for the S53 family protease's 513-loop is greater than −4.6, the enzymatic activity of the protease is increased relative to an enzyme including a 513-loop sequence as shown in SEQ ID NO: 1 and which has a hydrophobicity of about −4.6.

An additional aspect of the present disclosure provides an S53 family protease having an amino acid sequence that is at least 80% identical to SEQ ID NO: 1 and includes at least one mutation in the amino acid sequence's 513-loop which corresponds to amino acids 513 to 517 of SEQ ID NO: 1, wherein the at least one mutation is located at position 513, 514, 515, and/or 517, wherein when the substrate is casein and the hydrophobicity for the S53 family protease's 513-loop is greater than −4.6, the enzymatic activity of the protease is increased relative to an enzyme including a 513-loop sequence as shown in SEQ ID NO: 1 and which has a hydrophobicity of about −4.6.

In an aspect, the present disclosure provides an S53 family protease having an amino acid sequence that is at least 80% identical to SEQ ID NO: 1 and includes at least one mutation in the amino acid sequence's 513-loop which corresponds to amino acids 513 to 517 of SEQ ID NO: 1, wherein the at least one mutation is located at position 513, 514, 515, and/or 517, wherein when the substrate is zein and the hydrophobicity for the S53 family protease's 513-loop is greater than −4.6, the enzymatic activity of the protease is increased relative to an enzyme including a 513-loop sequence as shown in SEQ ID NO: 1 and which has a hydrophobicity of about −4.6.

In another aspect, the present disclosure provides an S53 family protease having an amino acid sequence that is at least 80% identical to SEQ ID NO: 1 and includes at least one mutation in the amino acid sequence's 513-loop which corresponds to amino acids 513 to 517 of SEQ ID NO: 1, wherein the at least one mutation is located at position 513, 514, 515, and/or 517, wherein when the substrate is pea protein and the hydrophobicity for the S53 family protease's 513-loop is greater than −4.6, the enzymatic activity of the protease is increased relative to an enzyme including a 513-loop sequence as shown in SEQ ID NO: 1 and which has a hydrophobicity of about −4.6.

In a further aspect, the present disclosure provides a food product including an S53 family protease having an amino acid sequence that is at least 80% identical to SEQ ID NO: 1 and includes at least one mutation in the amino acid sequence's 513-loop which corresponds to amino acids 513 to 517 of SEQ ID NO: 1 and a protein obtained from or naturally present in Adzuki beans, Almonds, Amaranth, Asparagus, Baby Lima, Barley, Beef, Black beans, Black eyed peas, Broccoli, Buckwheat, Cannellini beans, Cashews, Chia seeds, Chicken, Chickpea, Chlorella, Corn, Cowpea, Cranberry beans, Crowder pea, Egg, e.g., chicken egg, Fava beans, Field Peas, Flounder, Great Northern Beans, Green beans, Hemp protein powder, Kamut, Kidney beans, Lady cream peas, Lentil, Lupine Beans, Masoor Dal (Indian Red lentils), Milk or other milk products, e.g., cheese and yoghurt, Mung beans, Navy pea, Pea, Peanut, Pigeon Peas, Pink beans, Pinto beans, Pistachios, Pork, Quinoa, Royal Canin, Rye berries, Salmon, Soy, Spirulina, Sunflower seeds, Turkey, White beans, and Yellow split peas.

In an additional aspect, the present disclosure provides a food supplement including an S53 family protease having an amino acid sequence that is at least 80% identical to SEQ ID NO: 1 and includes at least one mutation in the amino acid sequence's 513-loop which corresponds to amino acids 513 to 517 of SEQ ID NO: 1.

In yet another aspect, the present disclosure provides a method for improving the digestion of proteins in a food product by a subject, the method including ingesting with the food product, a food supplement including an S53 family protease having an amino acid sequence that is at least 80% identical to SEQ ID NO: 1 and includes at least one mutation in the amino acid sequence's 513-loop which corresponds to amino acids 513 to 517 of SEQ ID NO: 1.

In yet a further aspect, the present disclosure provides a method for degrading a protein, the method including contacting the protein with an S53 family protease having an amino acid sequence that is at least 80% identical to SEQ ID NO: 1 and includes at least one mutation in the amino acid sequence's 513-loop which corresponds to amino acids 513 to 517 of SEQ ID NO: 1

In embodiments of any of the above aspects, the at least one mutation provides increased thermostability, increased enzymatic activity, and/or increased substrate specificity, relative to an S53 family protease having an amino acid sequence that lacks the at least one mutation located at position 513, 514, 515, and/or 517 with respect to SEQ ID NO: 1.

In embodiments of any of the above aspects, the S53 family protease includes at least two mutations in the 513-loop relative to SEQ ID NO: 1. In some cases, the at least two mutations are located at positions 513 and 514; at positions 513 and 515; at positions 513 and 517; at positions 514 and 515; at positions 514 and 517; or at positions 515 and 517.

In embodiments of any of the above aspects, the S53 family protease includes at least three mutations in the 513-loop relative to SEQ ID NO: 1. In some cases, the at least three mutations are located at positions 513, 515, and 517; at positions 513, 514, and 517; at positions 513, 514, and 515; or at positions 514, 515, and 517.

In embodiments of any of the above aspects, the S53 family protease includes at least four mutations in the 513-loop relative to SEQ ID NO: 1.

In embodiments of any of the above aspects, the S53 family protease includes five mutations in the 513-loop relative to SEQ ID NO: 1 which provide increased enzymatic activity and/or increased substrate specificity.

In embodiments of any of the above aspects, a mutation at position 513 includes an A to C substitution, A to D substitution, A to E substitution, A to F substitution, A to G substitution, A to H substitution, A to I substitution, A to K substitution, A to L substitution, A to M substitution, A to N substitution, A to P substitution, A to Q substitution, A to R substitution, A to S substitution, A to T substitution, A to V substitution, A to W substitution, or A to Y substitution.

In embodiments of any of the above aspects, a mutation at position 514 includes an N to A substitution, N to C substitution, N to D substitution, N to E substitution, N to F substitution, N to G substitution, N to H substitution, N to I substitution, N to K substitution, N to L substitution, N to M substitution, N to Q substitution, N to R substitution, N to S substitution, N to T substitution, N to V substitution, N to W substitution, or N to Y substitution.

In embodiments of any of the above aspects, a mutation at position 515 includes an R to A substitution, R to C substitution, R to D substitution, R to E substitution, R to F substitution, R to G substitution, R to I substitution, R to K substitution, R to L substitution, R to M substitution, R to N substitution, R to P substitution, R to Q substitution, R to S substitution, R to T substitution, R to V substitution, R to W substitution, or R to Y substitution.

In embodiments of any of the above aspects, a mutation at position 517 includes an R to A substitution, R to C substitution, R to D substitution, R to E substitution, R to F substitution, R to G substitution, R to H substitution, R to I substitution, R to K substitution, R to L substitution, R to M substitution, R to N substitution, R to P substitution, R to Q substitution, R to S substitution, R to T substitution, R to V substitution, R to W substitution, or R to Y substitution.

In embodiments of any of the above aspects, the at least one mutation in the amino acid sequence's 513-loop is as shown in one of Table 1 to Table 9.

Any protease, food product food supplement, or method disclosed herein is applicable to any herein-disclosed protease, food product food supplement, or method. In other words, any aspect or embodiment described herein can be combined with any other aspect or embodiment as disclosed herein.

### BRIEF DESCRIPTION OF THE DRAWINGS

FIG. 1 is a graph showing activity changes for various S53 mutants relative to the molecular weight of the amino acids in the A513 loop.

FIG. 2 is a graph showing activity changes for various S53 mutants relative to the molecular weight of the amino acids in the A513 loop and when the proteases were previously exposed to a heat treatment.

FIG. 3 is a graph showing activity changes for various S53 mutants relative to the hydrophobicity of the amino acids in the A513 loop.

FIG. 4 is a graph showing activity changes for various S53 mutants relative to the SVGER score of the amino acids in the A513 loop.

### DETAILED DESCRIPTION

The present disclosure provides proteolytic enzymes, e.g., S53 family proteases, having increased thermostability, increased enzymatic activity, and/or increased substrate specificity and which can digest a variety of food proteins, thereby enhancing a food's protein bioavailability.

In some embodiments, the S53 family proteases refer to and include proteases within and/or identified by MEROPS Accession MER0000995 (e.g., sedolisin, sedolisin-b, tripeptidyl-peptidase I, kumamolisin, kumamolisin-B, physarolisin, aorsin, physarolisin II, kumamolisin-As, grifolisin, scytalidolisin, among others). In some embodiments, the S53 protease is a pro-Kumamolisin. Pro-Kumamolisin generally refers to and includes the thermostable calcium-dependent endopeptidase derived from an acid/thermophilic Bacillus (Bacillus novosp. MN-32). In some embodiments, pro-Kumamolisin refers to and includes NCBI Gene ID: 18765799 (NCBI Reference Sequence XP_007297753.1, XM_007297691.1 to XP_007297753, and/or NW_006763082.1 (137488 . . . 139728); the contents of each of which is incorporated by reference in its entirety.

In various embodiments, the S53 family proteases of the present disclosure are variants of the protein having the amino acid sequence of SEQ ID NO: 1.

(SEQ ID NO: 1)
MSDMEKPWKEEEKREVLAGHARRQAPQAVDKGPVTGDQRISVTVVLRRQ
RGDELEAHVERQAALAPHARVHLEREAFAASHGASLDDFAEIRKFAEAH
GLTLDRAHVAAGTAVLSGPVDAVNQAFGVELRHFDHPDGSYRSYVGDVR
VPASIAPLIEAVLGLDTRPVARPHFRLRRRAEGEFEARSQSAAPTAYTP
LDVAQAYQFPEGLDGQGQCIAIIELGGGYDETSLAQYFASLGVSAPQVV
SVSVDGATNQPTGDPNGPDGEVELDIEVAGALAPGAKIAVYFAPNTDAG
FLNAITTAVHDPTHKPSIVSISWGGPEDSWAPASIAAMNRAFLDAAALG
VTVLAAAGDSGSTDGEQDGLYHVDFPAASPYVLACGGTRLVASAGRIER
ETVWNDGPDGGSTGGGVSRIFPLPSWQERANVPPSANPGAGSGRGVPDV
AGNADPATGYEVVIDGETTVIGGTSAVAPLFAALVARINQKLGKPVGYL
NPTLYQLPPEVFHDITEGNNDIANRARIYQAGPGWDPCTGLGSPIGIRL
LQALLPSASQAQP

In embodiments, the 513-loop sequence of an S53 protease lacking the at least one mutation comprises the sequence ANRAR (SEQ ID NO: 2) or ANRAQ (SEQ ID NO: 3).

### Illustrative S53 Family Proteases

An aspect of the present disclosure provides an S53 family protease having an amino acid sequence that is at least 80% identical to SEQ ID NO: 1 and includes at least one mutation in the amino acid sequence's 513-loop which corresponds to amino acids 513 to 517 of SEQ ID NO: 1, wherein the at least one mutation is located at position 513, 514, 515, and/or 517.

In embodiments, the at least one mutation provides increased thermostability, increased enzymatic activity, and/or increased substrate specificity, relative to an S53 family protease having an amino acid sequence that lacks the at least one mutation located at position 513, 514, 515, and/or 517 with respect to SEQ ID NO: 1.

In embodiments, the S53 family protease includes at least two mutations in the 513-loop relative to SEQ ID NO: 1. In some cases, the at least two mutations are located at positions 513 and 514; at positions 513 and 515; at positions 513 and 517; at positions 514 and 515; at positions 514 and 517; or at positions 515 and 517.

In embodiments, the S53 family protease includes at least three mutations in the 513-loop relative to SEQ ID NO: 1. In some cases, the at least three mutations are located at positions 513, 515, and 517; at positions 513, 514, and 517; at positions 513, 514, and 515; or at positions 514, 515, and 517.

In embodiments, the S53 family protease includes at least four mutations in the 513-loop relative to SEQ ID NO: 1.

In embodiments, the S53 family protease includes five mutations in the 513-loop relative to SEQ ID NO: 1 which provide increased enzymatic activity and/or increased substrate specificity.

In embodiments, a mutation at position 513 includes an A to C substitution, A to D substitution, A to E substitution, A to F substitution, A to G substitution, A to H substitution, A to I substitution, A to K substitution, A to L substitution, A to M substitution, A to N substitution, A to P substitution, A to Q substitution, A to R substitution, A to S substitution, A to T substitution, A to V substitution, A to W substitution, or A to Y substitution.

In embodiments, a mutation at position 514 includes an N to A substitution, N to C substitution, N to D substitution, N to E substitution, N to F substitution, N to G substitution, N to H substitution, N to I substitution, N to K substitution, N to L substitution, N to M substitution, N to Q substitution, N to R substitution, N to S substitution, N to T substitution, N to V substitution, N to W substitution, or N to Y substitution.

In embodiments, a mutation at position 515 includes an R to A substitution, R to C substitution, R to D substitution, R to E substitution, R to F substitution, R to G substitution, R to I substitution, R to K substitution, R to L substitution, R to M substitution, R to N substitution, R to P substitution, R to Q substitution, R to S substitution, R to T substitution, R to V substitution, R to W substitution, or R to Y substitution.

In embodiments, a mutation at position 517 includes an R to A substitution, R to C substitution, R to D substitution, R to E substitution, R to F substitution, R to G substitution, R to H substitution, R to I substitution, R to K substitution, R to L substitution, R to M substitution, R to N substitution, R to P substitution, R to Q substitution, R to S substitution, R to T substitution, R to V substitution, R to W substitution, or R to Y substitution.

In embodiments, the at least one mutation in the amino acid sequence's 513-loop is as shown in one of Table 1 to Table 9.

In embodiments, the S53 family protease further includes one or more additional mutations at position 13, 15, 18, 23, 35, 38, 40, 51, 52, 55, 64, 70, 73, 91, 96, 101, 121, 123, 143, 145, 147, 155, 174, 175, 181, 188, 198, 218, 228, 229, 236, 240, 243, 253, 261, 265, 268, 273, 280, 283, 286, 290, 292, 296, 297, 300, 303, 308, 312, 314, 319, 323, 324, 325, 326, 327, 328, 329, 330, 332, 341, 353, 359, 360, 371, 377, 385, 387, 390, 391, 392, 398, 404, 411, 417, 421, 422, 432, 433, 434, 441, 446, 448, 452, 459, 469, 474, 486, 499, 500, 501, 509, 513, 514, 515, 516, 517, 537, and/or 550 relative to SEQ ID NO: 1. In some cases, the one or more additional mutations includes substitutions selected from K13A, E15A, A18Q, R23A, T35A, Q38E, 140M, G51A, D52G, E55A, L641, V70E, E73K, 191 L, E96D, T101A, V121I, Q123R, V143L, D145E, R147T, L155M, L174M, R175Q, E181G, S188A, T228A, S236S, S240P, T253S, N261S, I283F, N297D, V303I, H308L, I312V, A325T, P326S, S328A, A387G, E391A, R392Q, S404A, S417A, R421H, G433S, V441 L, T459A, P486A, P499A, E500D, R517Q, 1537V, and/or A550P relative to SEQ ID NO: 1. In some cases, the one or more additional mutations includes substitutions selected from A325K; A325L; P326F; P326L; P326W; S328Q; S328H; E391V; E391L; E391I; E391M; S417W; V441W; T459W; T459R; P486Q; and/or R517G relative to SEQ ID NO: 1.

In embodiments, the present disclosure relates to an S53 family protease, further including one or more substrate selectivity mutations wherein the one or more substrate selectivity mutations increase protease substrate selectivity relative to a protease including an amino acid sequence as shown in SEQ ID NO: 1 and wherein the one or more additional mutations is at position 198, 218, 229, 243, 265, 268, 273, 280, 286, 290, 292, 296, 300, 314, 319, 323, 324, 325, 326, 327, 328, 329, 330, 332, 341, 353, 359, 360, 371, 377, 385, 390, 391, 398, 411, 417, 422, 432, 433, 434, 441, 446, 448, 452, 459, 469, 474, 486, 501, 509, 513, 514, 515, 516, and/or 517 relative to SEQ ID NO: 1.

In embodiments, the one or more substrate selectivity mutations includes substitutions selected from D198E, I218L, S229D, Q243G, G265A, E268M, E268T, E268C, E268H, E268Q, V273I, G280R, Y286K, N290T, N290S, D292F, D292Y, L296T, T300S, S314P, G319Q, S323R, W324K, A325K, A325L, P326F, P326W, P326L, A327Y, A327D, S328H, S328Q, I329L, A330F, M332I, M332L, A341 R, S353E, E359V, E359L, Q360C, A371S, A377G, A385V, A385L, I390W, E391V, E391M, E391 L, E391I, D398S, R411Q, R411M, R411E, R411D, S417W, A422V, A432G, G433G, S434I, S434R, S434T, S434V, V441W, D446S, A448N, E452Y, E452W, E452F, T459R, T459W, A469V, A474V, P486Q, V501C, N509H, A513T, A513D, A513Y, N514G, R515L, R515E, R515I, A516T, A516S, and/or R517G relative to SEQ ID NO: 1. In some cases, the S53 family protease further includes one or more substrate selectivity mutations wherein the one or more substrate selectivity mutations increase protease substrate selectivity when casein is a substrate relative to a protease including an amino acid sequence as shown in SEQ ID NO: 1 and wherein the one or more additional mutations is at position 198, 218, 229, 243, 265, 268, 273, 280, 286, 290, 292, 296, 300, 314, 319, 323, 324, 325, 326, 327, 328, 329, 330, 332, 341, 353, 359, 360, 371, 377, 385, 390, 391, 398, 411, 417, 422, 432, 433, 434, 441, 446, 448, 452, 459, 469, 474, 486, 501, 509, 513, 514, 515, 516, and/or 517 relative to SEQ ID NO: 1. In some cases, the one or more substrate selectivity mutations includes substitutions selected from D198E, I218L, S229D, Q243G, G265A, E268M, E268T, E268C, E268H, E268Q, V273I, G280R, Y286K, N290T, N290S, D292F, D292Y, L296T, T300S, S314P, G319Q, S323R, W324K, A325K, A325L, P326F, P326W, P326L, A327D, A327Y, S328Q, S328H, I329L, A330F, M332L, M332I, A341 R, S353E, E359L, E359V, Q360C, A371S, A377G, A385L, A385V, I390W, E391V, E391L, E391I, E391M, D398S, R411D, R411E, R411M, R411Q, S417W, A422V, A432G, G433G, S434I, S434R, S434T, S434V, V441W, D446S, A448N, A448Y, E452Y, E452W, E452F, T459R, T459W, A469V, A474V, P486Q, V501C, N509H, A513Y, A513D, A513T, N514G, R515I, R515E, R515L, A516S, A516T, and/or R517G relative to SEQ ID NO: 1.

In embodiments, the S53 family protease further includes one or more substrate selectivity mutations wherein the one or more substrate selectivity mutations increase protease substrate selectivity when zein is a substrate relative to a protease including an amino acid sequence as shown in SEQ ID NO: 1 and wherein the one or more additional mutations is at position 198, 218, 229, 243, 265, 268, 273, 280, 286, 290, 292, 296, 300, 314, 319, 323, 324, 325, 326, 327, 328, 329, 330, 332, 341, 353, 359, 360, 371, 377, 385, 390, 391, 398, 411, 417, 422, 432, 433, 434, 441, 446, 448, 452, 459, 469, 474, 486, 501, 509, 513, 514, 515, 516, and/or 517 relative to SEQ ID NO: 1. In some cases, the one or more substrate selectivity mutations includes substitutions selected from D198E, I218L, S229D, Q243G, G265A, E268M, E268T, E268C, E268H, E268Q, V273I, G280R, Y286K, N290T, N290S, D292Y, D292F, L296T, T300S, S314P, G319Q, S323R, W324K, A325K, A325L, P326F, P326L, P326W, A327D, A327Y, S328Q, S328H, I329L, A330F, M332I, M332L, A341 R, S353E, E359L, E359V, Q360C, A371S, A377G, A385L, A385V, I390W, E391V, E391M, E391I, E391L, D398S, R411Q, R411M, R411E, R411D, S417W, A422V, A432G, G433G, S434I, S434R, S434T, S434V, V441W, D446S, A448N, E452F, E452W, E452F, T459R, T459W, A469V, A474V, P486Q, V501 C, N509H, A513T, A513D, A513Y, N514G, R515I, R515E, R515L, A516S, A516T, and/or R517G relative to SEQ ID NO: 1.

In embodiments, the amino acid sequence lacks a mutation at position 198, 218, 229, 243, 265, 268, 273, 280, 286, 290, 292, 296, 300, 314, 319, 323, 324, 325, 326, 327, 328, 329, 330, 332, 341, 353, 359, 360, 371, 377, 385, 390, 391, 398, 411, 417, 422, 432, 433, 434, 441, 446, 448, 452, 459, 469, 474, 486, 501, and/or 509 relative to SEQ ID NO: 1.

In embodiments, the amino acid sequence lacks a mutation at position 266, 270, 352, and/or 466 relative to SEQ ID NO: 1.

In embodiments, the amino acid sequence lacks a mutation at each of positions 266, 270, 352, and 466 relative to SEQ ID NO: 1.

In embodiments, the amino acid sequence lacks a mutation position 516 relative to an amino acid SEQ ID NO: 1.

In embodiments, the amino acid sequence includes a mutation position 516 relative to an amino acid SEQ ID NO: 1. In some cases, the mutation at position 516 includes T.

In embodiments, the amino acid sequence is at least 85% identical to SEQ ID NO: 1, is at least 90% identical to SEQ ID NO: 1, is at least 95% identical to SEQ ID NO: 1, is at least 96% identical to SEQ ID NO: 1, is at least 97% identical to SEQ ID NO: 1, is at least 98% identical to SEQ ID NO: 1, or is at least 99% identical to SEQ ID NO: 1 and maintains or increases thermostability, enzymatic activity, and/or substrate specificity relative to an S53 family protease having the amino acid sequence of SEQ ID NO: 1.

In embodiments, the increased enzymatic activity occurs when the substrate is casein and wherein the at least one mutation in the amino acid sequence's 513-loop includes one or more substitutions selected from A513D, A513E, A513F, A513G, A513H, A513N, A513R, A513S, A513T, A513V, A513W, A513Y, N514A, N514C, N514D, N514E, N514F, N514G, N514N, N514Q, N514R, N514S, N514T, N514W, R515A, R515C, R515F, R515G, R515I, R515K, R515L, R515M, R515R, R515W, A516T, R515Y, R517A, R517D, R517F, R517G, R517H, R517K, R517L, R517M, R517N, R517P, R517R, R517T, R517W, or R517Y. In some cases, the at least one mutation in the amino acid sequence's 513-loop comprises a substitution selected from A513D, A513E, A513F, A513G, A513H, A513N, A513R, A513S, A513T, A513V, A513W, or A513Y; a substitution selected from N514A, N514C, N514D, N514E, N514F, N514G, N514N, N514Q, N514R, N514S, N514T, or N514W; a substitution selected from R515A, R515C, R515F, R515G, R515I, R515K, R515L, R515M, R515R, R515W, or R515Y; a A516T substitution; and/or a substitution selected from R517A, R517D, R517F, R517G, R517H, R517K, R517L, R517M, R517N, R517P, R517R, R517T, R517W, or R517Y. In some cases, the sequence for the amino acids of positions of 513 to 517 include one of ARWAM, ADYAG, ANRAR, DWIAD, DRLAG, ERYAH, ETAAR, FGWAT, FGLAR, FEFAD, FQYAG, FSCAR, GWMAG, HCWAF, NEWAA, RAWAL, SDRAW, TWRAT, VCWAN, WRWAY, WRWAY, WFCAG, WAKAR, WGGAW, WSAAR, WAIAK, WRYAP, YWRAG, YTCAR, YNRTR, or YAYAY.

In embodiments, the increased enzymatic activity occurs when the substrate is zein and wherein the at least one mutation in the amino acid sequence's 513-loop includes one or more substitutions selected from A513C, A513D, A513E, A513F, A513G, A513H, A513I, A513K, A513L, A513M, A513N, A513Q, A513R, A513S, A513T, A513V, A513W, A513Y, A516T, N514A, N514C, N514D, N514E, N514F, N514G, N514H, N5141, N514K, N514L, N514M, N514P, N514Q, N514R, N514S, N514T, N514V, N514W, N514Y, R515A, R515C, R515D, R515E, R515F, R515G, R515I, R515K, R515L, R515M, R515N, R515P, R515Q, R515S, R515T, R515V, R515W, R515Y, A516T, R517A, R517C, R517D, R517E, R517F, R517G, R517H, R517I, R517K, R517L, R517M, R517N, R517P, R517Q, R517S, R517T, R517V, R517W, or R517Y. In some cases, the at least one mutation in the amino acid sequence's 513-loop includes a substitution selected from A513C, A513D, A513E, A513F, A513G, A513H, A513I, A513K, A513L, A513M, A513N, A513Q, A513R, A513S, A513T, A513V, A513W, or A513Y; a substitution selected from N514A, N514C, N514D, N514E, N514F, N514G, N514H, N5141, N514K, N514L, N514M, N514P, N514Q, N514R, N514S, N514T, N514V, N514W, or N514Y; a substitution selected from R515A, R515C, R515D, R515E, R515F, R515G, R515I, R515K, R515L, R515M, R515N, R515P, R515Q, R515S, R515T, R515V, R515W, or R515Y; a A516T substitution; and/or a substitution selected from, R517A, R517C, R517D, R517E, R517F, R517G, R517H, R517I, R517K, R517L, R517M, R517N, R517P, R517Q, R517S, R517T, R517V, R517W, or R517Y. In some cases, the sequence for the amino acids of positions of 513 to 517 include one of ADYAG, AFLAE, AGGAS, ALGAL, ALIAN, ALVAG, APYAR, ARWAM, ASCAL, AYRAG, CCVAC, CLIAG, CSIAK, CTQAG, DGCAG, DGIAF, DGIAN, DHLAN, DRLAG, DRLAR, DTPAV, DWIAD, EFAAY, ELAAL, ENFAH, EQQAG, ERYAH, ETAAR, FEFAD, FGWAT, FHFAA, FHIAN, FQVAA, FQYAG, FRQAM, FSCAR, GMGAY, GRVAF, GWMAG, HCWAF, HDTAN, HFVAK, HNFAG, HSDAC, HYNAF, ILKAG, ILLAG, KNTAY, KTRAG, LCIAK, LDNAS, LFKAP, LFLAS, LLEAC, LRIAY, LRYAA, LTGAC, MCMAK, MDNAI, MLIAL, MLLAM, MLLAN, MQVAA, MSYAT, NDLAG, NECAY, NEWAA, NFSAL, NISAT, NSPAG, NVRAC, QFEAY, QGVAK, QMYAG, RAWAL, RGAAP, RRFAE, RSSAG, RSTAD, RYMAV, SDRAW, SGRAC, SGTAN, SIVAH, SMVAL, SNNAA, SSLAG, STDAG, SYRAD, TCFAE, TGGAG, TKTAW, TLIAQ, TLVAG, TMVAE, TVKAG, TWRAT, TYYAN, VCGAG, VCWAN, VFRAM, VNGAG, VRYAL, VYMAR, WAIAK, WAKAR, WFCAG, WGGAW, WMPAG, WRLAN, WRWAY, WRYAP, WSAAR, WSTAN, YAGAY, YALAY, YAYAY, YQVAL, YRNAN, YTCAR, or YWRAG.

In embodiments, the at least one mutation in the amino acid sequence's 513-loop increases thermostability as evidence by at least about 5% in enzymatic activity following heat treatment relative to an enzyme including a 513-loop sequence as shown in SEQ ID NO: 1.

In embodiments, the 513-loop sequence of an S53 protease lacking the at least one mutation comprises the sequence ANRAR (SEQ ID NO: 2) or ANRAQ (SEQ ID NO: 3).

In embodiments, the increased thermostability occurs when the substrate is casein and wherein the at least one mutation in the amino acid sequence's 513-loop includes one or more substitutions selected from A513C, A513D, A513E, A513F, A513G, A513H, A513I, A513K, A513L, A513M, A513N, A513P, A513Q, A513R, A515S, A513T, A513V, A513W, A513W, N514C, N514D, N514F, N514G, N514H, N5141, N514L, N514M, N514Q, N514R, N514S, N514T, N514V, R515A, R515C, R515D, R515E, R515F, R515G, R515I, R515K, R515L, R515M, R515P, R515Q, R515S, R515T, R515V, R515W, R515Y, A516T, R517A, R517C, R517E, R517F, R517G, R517K, R517L, R517M, R517N, R517P, R517S, R517T, or R517Y. In some cases, the at least one mutation in the amino acid sequence's 513-loop includes a set of substitutions selected from a substitution selected from A513C, A513D, A513E, A513F, A513G, A513H, A513I, A513K, A513L, A513M, A513N, A513P, A513Q, A513R, A515S, A513T, A513V, A513W, or A513W; a substitution selected from N514C, N514D, N514F, N514G, N514H, N5141, N514L, N514M, N514Q, N514R, N514S, N514T, or N514V; a substitution selected from R515A, R515C, R515D, R515E, R515F, R515G, R515I, R515K, R515L, R515M, R515P, R515Q, R515S, R515T, R515V, R515W, or R515Y; a A516T substitution; and/or a substitution selected from R517A, R517C, R517E, R517F, R517G, R517K, R517L, R517M, R517N, R517P, R517S, R517T, or R517Y. In some cases, the sequence for the amino acids of positions of 513 to 517 include one of WRWAY, FQYAG, SSLAG, QMYAG, NISAT, PRCAE, LTGAC, YNRTR, FHFAA, TLVAG, FHIAN, YQVAL, HNFAG, DRLAG, VCGAG, HCWAF, LRIAY, RSSAG, STDAG, NDLAG, FRQAM, TVKAG, CLIAG, NSPAG, SGRAC, VNGAG, ALVAG, CTQAG, DGIAF, TCFAE, ILLAG, MQVAA, EQQAG, MLIAL, ELAAL, TMVAE, ALIAN, LLEAC, MLLAN, KTRAG, LCIAK, FQVAA, CCVAC, MCMAK, QGVAK, LFLAS, MLLAM, DGIAN, ALGAL, RGAAP, TGGAG, ILKAG, ASCAL, KNTAY, DHLAN, AFLAE, EFAAY, NVRAC, GRVAF, and SMVAL.

In embodiments, the increased thermostability as occurs when the substrate is a pea protein and wherein the at least one mutation in the amino acid sequence's 513-loop includes one or more substitutions selected from A513C, A513D, A513E, A513F, A513G, A513H, A513I, A513K, A513L, A513M, A513N, A513Q, A513S, A513T, A513W, A513Y, N514A, N514C, N514D, N514F, N514G, N5141, N514K, N514L, N514M, N514N, N514P, N514Q, N514R, N514T, N514V, N514W, N514Y, R515A, R515C, R515F, R515G, R515M, R515N, R515Q, R515T, R515V, R515W, R517A, R517C, R517E, R517F, R517G, R517I, R517K, R517L, R517M, R517N, or R517Y. In some cases, the at least one mutation in the amino acid sequence's 513-loop includes a substitution selected from A513C, A513D, A513E, A513F, A513G, A513H, A513I, A513K, A513L, A513M, A513N, A513Q, A513S, A513T, A513W, or A513Y; a substitution selected from N514A, N514C, N514D, N514F, N514G, N5141, N514K, N514L, N514M, N514N, N514P, N514Q, N514R, N514T, N514V, N514W, or N514Y; a substitution selected from R515A, R515C, R515F, R515G, R515M, R515N, R515Q, R515T, R515V, or R515W; a A516T substitution; and/or a substitution selected from R517A, R517C, R517E, R517F, R517G, R517I, R517K, R517L, R517M, R517N, or R517Y. In some cases, the sequence for the amino acids of positions of 513 to 517 include one of YNRTR, SSLAG, STDAG, VNGAG, TGGAG, DRLAG, TLVAG, FRQAM, LTGAC, LCIAK, CTQAG, HNFAG, FHIAN, QGVAK, QMYAG, TVKAG, EQQAG, NDLAG, SGRAC, SMVAL, ALVAG, MCMAK, GRVAF, FQYAG, MQVAA, NSPAG, ILLAG, FQVAA, MLLAN, YQVAL, EFAAY, FHFAA, ALGAL, DGIAF, ILKAG, ANRAR, KANAC, DGIAN, KNTAY, KTRAG, ALIAN, NVRAC, SNNAA, HCWAF, ASCAL, CLIAG, WRWAY, TCFAE, DHLAN, MLIAL, MLLAM, AFLAE, LFLAS, CCVAC, or MDNAI.

In embodiments, the at least one mutation in the amino acid sequence's 513-loop increases substrate specificity by at least about 5% relative to an enzyme including a 513-loop sequence as shown in SEQ ID NO: 1.

In embodiments, the 513-loop sequence of an S53 protease lacking the at least one mutation comprises the sequence ANRAR (SEQ ID NO: 2) or ANRAQ (SEQ ID NO: 3).

In embodiments, the increased substrate specificity occurs when the substrate is zein and wherein the sequence for the amino acids of positions of 513 to 517 include WRWAY.

In embodiments, the increased substrate specificity occurs when the substrate is casein and wherein the sequence for the amino acids of positions of 513 to 517 include WRWAY.

In embodiments, the increased substrate specificity occurs when the substrate is casein and wherein the sequence for the amino acids of positions of 513 to 517 include WRWAY.

In embodiments, when the substrate is casein and the molecular weight for the S53 family protease's 513-loop is greater than 586 kDa, the enzymatic activity of the protease is increased relative to an enzyme including a 513-loop sequence as shown in SEQ ID NO: 1 and which has a molecular weight of about 580 kDa.

In embodiments, when the substrate is casein and the hydrophobicity for the S53 family protease's 513-loop is greater than −4.6, the enzymatic activity of the protease is increased relative to an enzyme including a 513-loop sequence as shown in SEQ ID NO: 1 and which has a hydrophobicity of about −4.6.

In embodiments, when substrate is casein and the S53 family protease includes an SVGER score of greater than −2.2, the enzymatic activity of the protease is increased relative to an enzyme including an amino acid sequence as shown in SEQ ID NO: 1 and which has a SVGER score of about −2.2.

In embodiments, when the substrate is zein and the molecular weight for the S53 family protease's 513-loop is greater than 586 kDa, the enzymatic activity of the protease is increased relative to an enzyme including a 513-loop sequence as shown in SEQ ID NO: 1 and which has a molecular weight of about 580 kDa.

In embodiments, when the substrate is zein and the hydrophobicity for the S53 family protease's 513-loop ranges is greater than −4.6 the enzymatic activity of the protease is increased relative to an enzyme including a 513-loop sequence as shown in SEQ ID NO: 1 and which has a hydrophobicity of about −4.6.

In embodiments, when substrate is zein and the S53 family protease includes an SVGER score of greater than −2.2, the enzymatic activity of the protease is increased relative to an enzyme including an amino acid sequence as shown in SEQ ID NO: 1 and which has a SVGER score of about −2.2.

In embodiments, when the substrate is pea protein and the molecular weight for the S53 family protease's 513-loop is greater than 586 kDa, the enzymatic activity of the protease is increased relative to an enzyme including a 513-loop sequence as shown in SEQ ID NO: 1 and which has a molecular weight of about 580 kDa.

In embodiments, when the substrate is pea protein and the hydrophobicity for the S53 family protease's 513-loop is greater than −4.6, the enzymatic activity of the protease is increased relative to an enzyme including a 513-loop sequence as shown in SEQ ID NO: 1 and which has a hydrophobicity of about −4.6.

In embodiments, when substrate is pea protein and the S53 family protease includes an SVGER score of greater than −2.2, the enzymatic activity the enzymatic activity of the protease is increased relative to an enzyme including an amino acid sequence as shown in SEQ ID NO: 1 and which has a SVGER score of about −2.2.

In embodiments, the substrate for the protease is a protein obtained from or naturally present in Adzuki beans, Almonds, Amaranth, Asparagus, Baby Lima, Barley, Beef, Black beans, Black eyed peas, Broccoli, Buckwheat, Cannellini beans, Cashews, Chia seeds, Chicken, Chickpea, Chlorella, Corn, Cowpea, Cranberry beans, Crowder pea, Egg, e.g., chicken egg, Fava beans, Field Peas, Flounder, Great Northern Beans, Green beans, Hemp protein powder, Kamut, Kidney beans, Lady cream peas, Lentil, Lupine Beans, Masoor Dal (Indian Red lentils), Milk or other milk products, e.g., cheese and yoghurt, Mung beans, Navy pea, Pea, Peanut, Pigeon Peas, Pink beans, Pinto beans, Pistachios, Pork, Quinoa, Royal Canin, Rye berries, Salmon, Soy, Spirulina, Sunflower seeds, Turkey, White beans, and Yellow split peas.

In embodiments, the protein is obtained from or naturally present in corn, optionally, wherein the protein is zein.

In embodiments, the protein is obtained from or naturally present in milk or a milk product, optionally, wherein the protein is casein.

In embodiments, the protein is obtained from or naturally present in a pea.

### Additional Illustrative S53 Family Proteases

An aspect of the present disclosure provides an S53 family protease having an amino acid sequence that is identical to SEQ ID NO: 1 except it includes at least one mutation in the amino acid sequence's 513-loop which corresponds to amino acids 513 to 517 of SEQ ID NO: 1, wherein the at least one mutation is located at position 513, 514, 515, and/or 517.

In embodiments, the at least one mutation provides increased thermostability, increased enzymatic activity, and/or increased substrate specificity, relative to an S53 family protease having an amino acid sequence that lacks the at least one mutation located at position 513, 514, 515, and/or 517 with respect to SEQ ID NO: 1.

In embodiments, the S53 family protease includes at least two mutations in the 513-loop relative to SEQ ID NO: 1. In some cases, the at least two mutations are located at positions 513 and 514; at positions 513 and 515; at positions 513 and 517; at positions 514 and 515; at positions 514 and 517; or at positions 515 and 517.

In embodiments, the S53 family protease includes at least three mutations in the 513-loop relative to SEQ ID NO: 1. In some cases, the at least three mutations are located at positions 513, 515, and 517; at positions 513, 514, and 517; at positions 513, 514, and 515; or at positions 514, 515, and 517.

In embodiments, the S53 family protease includes at least four mutations in the 513-loop relative to SEQ ID NO: 1.

In embodiments, the S53 family protease includes five mutations in the 513-loop relative to SEQ ID NO: 1.

In embodiments, a mutation at position 513 includes an A to C substitution, A to D substitution, A to E substitution, A to F substitution, A to G substitution, A to H substitution, A to I substitution, A to K substitution, A to L substitution, A to M substitution, A to N substitution, A to P substitution, A to Q substitution, A to R substitution, A to S substitution, A to T substitution, A to V substitution, A to W substitution, or A to Y substitution.

In embodiments, a mutation at position 514 includes an N to A substitution, N to C substitution, N to D substitution, N to E substitution, N to F substitution, N to G substitution, N to H substitution, N to I substitution, N to K substitution, N to L substitution, N to M substitution, N to Q substitution, N to R substitution, N to S substitution, N to T substitution, N to V substitution, N to W substitution, or N to Y substitution.

In embodiments, a mutation at position 515 includes an R to A substitution, R to C substitution, R to D substitution, R to E substitution, R to F substitution, R to G substitution, R to I substitution, R to K substitution, R to L substitution, R to M substitution, R to N substitution, R to P substitution, R to Q substitution, R to S substitution, R to T substitution, R to V substitution, R to W substitution, or R to Y substitution.

In embodiments, a mutation at position 517 includes an R to A substitution, R to C substitution, R to D substitution, R to E substitution, R to F substitution, R to G substitution, R to H substitution, R to I substitution, R to K substitution, R to L substitution, R to M substitution, R to N substitution, R to P substitution, R to Q substitution, R to S substitution, R to T substitution, R to V substitution, R to W substitution, or R to Y substitution.

In embodiments, the at least one mutation in the amino acid sequence's 513-loop is as shown in one of Table 1 to Table 9.

In embodiments, the at least one mutation in the amino acid sequence's 513-loop increases enzymatic activity by at least about 5% relative to an enzyme including a 513-loop sequence as shown in SEQ ID NO: 1.

In embodiments, the 513-loop sequence of an S53 protease lacking the at least one mutation comprises the sequence ANRAR (SEQ ID NO: 2) or ANRAQ (SEQ ID NO: 3).

In embodiments, the increased enzymatic activity occurs when the substrate is casein and wherein the at least one mutation in the amino acid sequence's 513-loop includes one or more substitutions selected from A513D, A513E, A513F, A513G, A513H, A513N, A513R, A513S, A513T, A513V, A513W, A513Y, N514A, N514C, N514D, N514E, N514F, N514G, N514N, N514Q, N514R, N514S, N514T, N514W, R515A, R515C, R515F, R515G, R515I, R515K, R515L, R515M, R515R, R515W, A516T, R515Y, R517A, R517D, R517F, R517G, R517H, R517K, R517L, R517M, R517N, R517P, R517R, R517T, R517W, or R517Y. In some cases, the at least one mutation in the amino acid sequence's 513-loop comprises a substitution selected from A513D, A513E, A513F, A513G, A513H, A513N, A513R, A513S, A513T, A513V, A513W, or A513Y; a substitution selected from N514A, N514C, N514D, N514E, N514F, N514G, N514N, N514Q, N514R, N514S, N514T, or N514W; a substitution selected from R515A, R515C, R515F, R515G, R515I, R515K, R515L, R515M, R515R, R515W, or R515Y; a A516T substitution; and/or a substitution selected from R517A, R517D, R517F, R517G, R517H, R517K, R517L, R517M, R517N, R517P, R517R, R517T, R517W, or R517Y. In some cases, the sequence for the amino acids of positions of 513 to 517 include one of ARWAM, ADYAG, ANRAR, DWIAD, DRLAG, ERYAH, ETAAR, FGWAT, FGLAR, FEFAD, FQYAG, FSCAR, GWMAG, HCWAF, NEWAA, RAWAL, SDRAW, TWRAT, VCWAN, WRWAY, WRWAY, WFCAG, WAKAR, WGGAW, WSAAR, WAIAK, WRYAP, YWRAG, YTCAR, YNRTR, or YAYAY.

In embodiments, the increased enzymatic activity occurs when the substrate is zein and wherein the at least one mutation in the amino acid sequence's 513-loop includes one or more substitutions selected from A513C, A513D, A513E, A513F, A513G, A513H, A513I, A513K, A513L, A513M, A513N, A513Q, A513R, A513S, A513T, A513V, A513W, A513Y, A516T, N514A, N514C, N514D, N514E, N514F, N514G, N514H, N5141, N514K, N514L, N514M, N514P, N514Q, N514R, N514S, N514T, N514V, N514W, N514Y, R515A, R515C, R515D, R515E, R515F, R515G, R515I, R515K, R515L, R515M, R515N, R515P, R515Q, R515S, R515T, R515V, R515W, R515Y, A516T, R517A, R517C, R517D, R517E, R517F, R517G, R517H, R517I, R517K, R517L, R517M, R517N, R517P, R517Q, R517S, R517T, R517V, R517W, or R517Y. In some cases, the at least one mutation in the amino acid sequence's 513-loop includes a substitution selected from A513C, A513D, A513E, A513F, A513G, A513H, A513I, A513K, A513L, A513M, A513N, A513Q, A513R, A513S, A513T, A513V, A513W, or A513Y; a substitution selected from N514A, N514C, N514D, N514E, N514F, N514G, N514H, N5141, N514K, N514L, N514M, N514P, N514Q, N514R, N514S, N514T, N514V, N514W, or N514Y; a substitution selected from R515A, R515C, R515D, R515E, R515F, R515G, R515I, R515K, R515L, R515M, R515N, R515P, R515Q, R515S, R515T, R515V, R515W, or R515Y; a A516T substitution; and/or a substitution selected from, R517A, R517C, R517D, R517E, R517F, R517G, R517H, R517I, R517K, R517L, R517M, R517N, R517P, R517Q, R517S, R517T, R517V, R517W, or R517Y. In some cases, the sequence for the amino acids of positions of 513 to 517 include one of ADYAG, AFLAE, AGGAS, ALGAL, ALIAN, ALVAG, APYAR, ARWAM, ASCAL, AYRAG, CCVAC, CLIAG, CSIAK, CTQAG, DGCAG, DGIAF, DGIAN, DHLAN, DRLAG, DRLAR, DTPAV, DWIAD, EFAAY, ELAAL, ENFAH, EQQAG, ERYAH, ETAAR, FEFAD, FGWAT, FHFAA, FHIAN, FQVAA, FQYAG, FRQAM, FSCAR, GMGAY, GRVAF, GWMAG, HCWAF, HDTAN, HFVAK, HNFAG, HSDAC, HYNAF, ILKAG, ILLAG, KNTAY, KTRAG, LCIAK, LDNAS, LFKAP, LFLAS, LLEAC, LRIAY, LRYAA, LTGAC, MCMAK, MDNAI, MLIAL, MLLAM, MLLAN, MQVAA, MSYAT, NDLAG, NECAY, NEWAA, NFSAL, NISAT, NSPAG, NVRAC, QFEAY, QGVAK, QMYAG, RAWAL, RGAAP, RRFAE, RSSAG, RSTAD, RYMAV, SDRAW, SGRAC, SGTAN, SIVAH, SMVAL, SNNAA, SSLAG, STDAG, SYRAD, TCFAE, TGGAG, TKTAW, TLIAQ, TLVAG, TMVAE, TVKAG, TWRAT, TYYAN, VCGAG, VCWAN, VFRAM, VNGAG, VRYAL, VYMAR, WAIAK, WAKAR, WFCAG, WGGAW, WMPAG, WRLAN, WRWAY, WRYAP, WSAAR, WSTAN, YAGAY, YALAY, YAYAY, YQVAL, YRNAN, YTCAR, or YWRAG.

In embodiments, the at least one mutation in the amino acid sequence's 513-loop increases thermostability as evidence by at least about 5% in enzymatic activity following heat treatment relative to an enzyme including a 513-loop sequence as shown in SEQ ID NO: 1.

In embodiments, the 513-loop sequence of an S53 protease lacking the at least one mutation comprises the sequence ANRAR (SEQ ID NO: 2) or ANRAQ (SEQ ID NO: 3).

In embodiments, the increased thermostability occurs when the substrate is casein and wherein the at least one mutation in the amino acid sequence's 513-loop includes one or more substitutions selected from A513C, A513D, A513E, A513F, A513G, A513H, A513I, A513K, A513L, A513M, A513N, A513P, A513Q, A513R, A515S, A513T, A513V, A513W, A513W, N514C, N514D, N514F, N514G, N514H, N5141, N514L, N514M, N514Q, N514R, N514S, N514T, N514V, R515A, R515C, R515D, R515E, R515F, R515G, R515I, R515K, R515L, R515M, R515P, R515Q, R515S, R515T, R515V, R515W, R515Y, A516T, R517A, R517C, R517E, R517F, R517G, R517K, R517L, R517M, R517N, R517P, R517S, R517T, or R517Y. In some cases, the at least one mutation in the amino acid sequence's 513-loop includes a set of substitutions selected from a substitution selected from A513C, A513D, A513E, A513F, A513G, A513H, A513I, A513K, A513L, A513M, A513N, A513P, A513Q, A513R, A515S, A513T, A513V, A513W, or A513W; a substitution selected from N514C, N514D, N514F, N514G, N514H, N5141, N514L, N514M, N514Q, N514R, N514S, N514T, or N514V; a substitution selected from R515A, R515C, R515D, R515E, R515F, R515G, R515I, R515K, R515L, R515M, R515P, R515Q, R515S, R515T, R515V, R515W, or R515Y; a A516T substitution; and/or a substitution selected from R517A, R517C, R517E, R517F, R517G, R517K, R517L, R517M, R517N, R517P, R517S, R517T, or R517Y. In some cases, the sequence for the amino acids of positions of 513 to 517 include one of WRWAY, FQYAG, SSLAG, QMYAG, NISAT, PRCAE, LTGAC, YNRTR, FHFAA, TLVAG, FHIAN, YQVAL, HNFAG, DRLAG, VCGAG, HCWAF, LRIAY, RSSAG, STDAG, NDLAG, FRQAM, TVKAG, CLIAG, NSPAG, SGRAC, VNGAG, ALVAG, CTQAG, DGIAF, TCFAE, ILLAG, MQVAA, EQQAG, MLIAL, ELAAL, TMVAE, ALIAN, LLEAC, MLLAN, KTRAG, LCIAK, FQVAA, CCVAC, MCMAK, QGVAK, LFLAS, MLLAM, DGIAN, ALGAL, RGAAP, TGGAG, ILKAG, ASCAL, KNTAY, DHLAN, AFLAE, EFAAY, NVRAC, GRVAF, and SMVAL.

In embodiments, the increased thermostability as occurs when the substrate is a pea protein and wherein the at least one mutation in the amino acid sequence's 513-loop includes one or more substitutions selected from A513C, A513D, A513E, A513F, A513G, A513H, A513I, A513K, A513L, A513M, A513N, A513Q, A513S, A513T, A513W, A513Y, N514A, N514C, N514D, N514F, N514G, N5141, N514K, N514L, N514M, N514N, N514P, N514Q, N514R, N514T, N514V, N514W, N514Y, R515A, R515C, R515F, R515G, R515M, R515N, R515Q, R515T, R515V, R515W, R517A, R517C, R517E, R517F, R517G, R517I, R517K, R517L, R517M, R517N, or R517Y. In some cases, the at least one mutation in the amino acid sequence's 513-loop includes a substitution selected from A513C, A513D, A513E, A513F, A513G, A513H, A513I, A513K, A513L, A513M, A513N, A513Q, A513S, A513T, A513W, or A513Y; a substitution selected from N514A, N514C, N514D, N514F, N514G, N5141, N514K, N514L, N514M, N514N, N514P, N514Q, N514R, N514T, N514V, N514W, or N514Y; a substitution selected from R515A, R515C, R515F, R515G, R515M, R515N, R515Q, R515T, R515V, or R515W; a A516T substitution; and/or a substitution selected from R517A, R517C, R517E, R517F, R517G, R517I, R517K, R517L, R517M, R517N, or R517Y. In some cases, the sequence for the amino acids of positions of 513 to 517 include one of YNRTR, SSLAG, STDAG, VNGAG, TGGAG, DRLAG, TLVAG, FRQAM, LTGAC, LCIAK, CTQAG, HNFAG, FHIAN, QGVAK, QMYAG, TVKAG, EQQAG, NDLAG, SGRAC, SMVAL, ALVAG, MCMAK, GRVAF, FQYAG, MQVAA, NSPAG, ILLAG, FQVAA, MLLAN, YQVAL, EFAAY, FHFAA, ALGAL, DGIAF, ILKAG, ANRAR, KANAC, DGIAN, KNTAY, KTRAG, ALIAN, NVRAC, SNNAA, HCWAF, ASCAL, CLIAG, WRWAY, TCFAE, DHLAN, MLIAL, MLLAM, AFLAE, LFLAS, CCVAC, or MDNAI.

In embodiments, the at least one mutation in the amino acid sequence's 513-loop increases substrate specificity by at least about 5% relative to an enzyme including a 513-loop sequence as shown in SEQ ID NO: 1.

In embodiments, the 513-loop sequence of an S53 protease lacking the at least one mutation comprises the sequence ANRAR (SEQ ID NO: 2) or ANRAQ (SEQ ID NO: 3).

In embodiments, the increased substrate specificity occurs when the substrate is zein and wherein the sequence for the amino acids of positions of 513 to 517 include WRWAY.

In embodiments, the increased substrate specificity occurs when the substrate is casein and wherein the sequence for the amino acids of positions of 513 to 517 include WRWAY.

In embodiments, the increased substrate specificity occurs when the substrate is casein and wherein the sequence for the amino acids of positions of 513 to 517 include WRWAY.

In embodiments, when the substrate is casein and the molecular weight for the S53 family protease's 513-loop is greater than 586 kDa, the enzymatic activity of the protease is increased relative to an enzyme including a 513-loop sequence as shown in SEQ ID NO: 1 and which has a molecular weight of about 580 kDa.

In embodiments, when the substrate is casein and the hydrophobicity for the S53 family protease's 513-loop is greater than −4.6, the enzymatic activity of the protease is increased relative to an enzyme including a 513-loop sequence as shown in SEQ ID NO: 1 and which has a hydrophobicity of about −4.6.

In embodiments, when substrate is casein and the S53 family protease includes an SVGER score of greater than −2.2, the enzymatic activity of the protease is increased relative to an enzyme including an amino acid sequence as shown in SEQ ID NO: 1 and which has a SVGER score of about −2.2.

In embodiments, when the substrate is zein and the molecular weight for the S53 family protease's 513-loop is greater than 586 kDa, the enzymatic activity of the protease is increased relative to an enzyme including a 513-loop sequence as shown in SEQ ID NO: 1 and which has a molecular weight of about 580 kDa.

In embodiments, when the substrate is zein and the hydrophobicity for the S53 family protease's 513-loop ranges is greater than −4.6 the enzymatic activity of the protease is increased relative to an enzyme including a 513-loop sequence as shown in SEQ ID NO: 1 and which has a hydrophobicity of about −4.6.

In embodiments, when substrate is zein and the S53 family protease includes an SVGER score of greater than −2.2, the enzymatic activity of the protease is increased relative to an enzyme including an amino acid sequence as shown in SEQ ID NO: 1 and which has a SVGER score of about −2.2.

In embodiments, when the substrate is pea protein and the molecular weight for the S53 family protease's 513-loop is greater than 586 kDa, the enzymatic activity of the protease is increased relative to an enzyme including a 513-loop sequence as shown in SEQ ID NO: 1 and which has a molecular weight of about 580 kDa.

In embodiments, when the substrate is pea protein and the hydrophobicity for the S53 family protease's 513-loop is greater than −4.6, the enzymatic activity of the protease is increased relative to an enzyme including a 513-loop sequence as shown in SEQ ID NO: 1 and which has a hydrophobicity of about −4.6.

In embodiments, when substrate is pea protein and the S53 family protease includes an SVGER score of greater than −2.2, the enzymatic activity the enzymatic activity of the protease is increased relative to an enzyme including an amino acid sequence as shown in SEQ ID NO: 1 and which has a SVGER score of about −2.2.

In embodiments, the substrate for the protease is a protein obtained from or naturally present in Adzuki beans, Almonds, Amaranth, Asparagus, Baby Lima, Barley, Beef, Black beans, Black eyed peas, Broccoli, Buckwheat, Cannellini beans, Cashews, Chia seeds, Chicken, Chickpea, Chlorella, Corn, Cowpea, Cranberry beans, Crowder pea, Egg, e.g., chicken egg, Fava beans, Field Peas, Flounder, Great Northern Beans, Green beans, Hemp protein powder, Kamut, Kidney beans, Lady cream peas, Lentil, Lupine Beans, Masoor Dal (Indian Red lentils), Milk or other milk products, e.g., cheese and yoghurt, Mung beans, Navy pea, Pea, Peanut, Pigeon Peas, Pink beans, Pinto beans, Pistachios, Pork, Quinoa, Royal Canin, Rye berries, Salmon, Soy, Spirulina, Sunflower seeds, Turkey, White beans, and Yellow split peas.

In embodiments, the protein is obtained from or naturally present in corn, optionally, wherein the protein is zein.

In embodiments, the protein is obtained from or naturally present in milk or a milk product, optionally, wherein the protein is casein.

In embodiments, the protein is obtained from or naturally present in a pea.

### Illustrative S53 Family Proteases Having Improved Hydrophobicity

A surprising aspect of the present disclosure is the improved properties provided by increased hydrophobicity for the S53 family protease's 513-loop. Without wishing to be bound by theory, but many substates for the S53 family protease are hydrophobic. Thus, by having increased hydrophobicity, at least, in the protease's 513-loop may help the enzyme contact, interact with, and the like, its substrate and thereby enhancing the protease's enzymatic activity. Indeed, the more hydrophobic the amino substitutions in the 513-loop, the greater the enzymatic activity.

An aspect of the present disclosure provides an S53 family protease having an amino acid sequence that is at least 80% identical to SEQ ID NO: 1 and includes at least one mutation in the amino acid sequence's 513-loop which corresponds to amino acids 513 to 517 of SEQ ID NO: 1, wherein the at least one mutation is located at position 513, 514, 515, and/or 517, wherein the hydrophobicity for the S53 family protease's 513-loop is greater than −4.6, the enzymatic activity of the protease is increased relative to an enzyme including a 513-loop sequence as shown in SEQ ID NO: 1 and which has a hydrophobicity of about −4.6.

An aspect of the present disclosure provides an S53 family protease having an amino acid sequence that is at least 80% identical to SEQ ID NO: 1 and includes at least one mutation in the amino acid sequence's 513-loop which corresponds to amino acids 513 to 517 of SEQ ID NO: 1, wherein the at least one mutation is located at position 513, 514, 515, and/or 517, wherein when the substrate is casein and the hydrophobicity for the S53 family protease's 513-loop is greater than −4.6, the enzymatic activity of the protease is increased relative to an enzyme including a 513-loop sequence as shown in SEQ ID NO: 1 and which has a hydrophobicity of about −4.6.

An aspect of the present disclosure provides an S53 family protease having an amino acid sequence that is at least 80% identical to SEQ ID NO: 1 and includes at least one mutation in the amino acid sequence's 513-loop which corresponds to amino acids 513 to 517 of SEQ ID NO: 1, wherein the at least one mutation is located at position 513, 514, 515, and/or 517, wherein when the substrate is zein and the hydrophobicity for the S53 family protease's 513-loop is greater than −4.6, the enzymatic activity of the protease is increased relative to an enzyme including a 513-loop sequence as shown in SEQ ID NO: 1 and which has a hydrophobicity of about −4.6.

An aspect of the present disclosure provides an S53 family protease having an amino acid sequence that is at least 80% identical to SEQ ID NO: 1 and includes at least one mutation in the amino acid sequence's 513-loop which corresponds to amino acids 513 to 517 of SEQ ID NO: 1, wherein the at least one mutation is located at position 513, 514, 515, and/or 517, wherein when the substrate is pea protein and the hydrophobicity for the S53 family protease's 513-loop is greater than −4.6, the enzymatic activity of the protease is increased relative to an enzyme including a 513-loop sequence as shown in SEQ ID NO: 1 and which has a hydrophobicity of about −4.6.

In embodiments, the at least one mutation provides increased thermostability, increased enzymatic activity, and/or increased substrate specificity, relative to an S53 family protease having an amino acid sequence that lacks the at least one mutation located at position 513, 514, 515, and/or 517 with respect to SEQ ID NO: 1.

In embodiments, the S53 family protease includes at least two mutations in the 513-loop relative to SEQ ID NO: 1. In some cases, the at least two mutations are located at positions 513 and 514; at positions 513 and 515; at positions 513 and 517; at positions 514 and 515; at positions 514 and 517; or at positions 515 and 517.

In embodiments, the S53 family protease includes at least three mutations in the 513-loop relative to SEQ ID NO: 1. In some cases, the at least three mutations are located at positions 513, 515, and 517; at positions 513, 514, and 517; at positions 513, 514, and 515; or at positions 514, 515, and 517.

In embodiments, the S53 family protease includes at least four mutations in the 513-loop relative to SEQ ID NO: 1.

In embodiments, the S53 family protease includes five mutations in the 513-loop relative to SEQ ID NO: 1 which provide increased enzymatic activity and/or increased substrate specificity.

In embodiments, a mutation at position 513 includes an A to C substitution, A to D substitution, A to E substitution, A to F substitution, A to G substitution, A to H substitution, A to I substitution, A to K substitution, A to L substitution, A to M substitution, A to N substitution, A to P substitution, A to Q substitution, A to R substitution, A to S substitution, A to T substitution, A to V substitution, A to W substitution, or A to Y substitution.

In embodiments, a mutation at position 514 includes an N to A substitution, N to C substitution, N to D substitution, N to E substitution, N to F substitution, N to G substitution, N to H substitution, N to I substitution, N to K substitution, N to L substitution, N to M substitution, N to Q substitution, N to R substitution, N to S substitution, N to T substitution, N to V substitution, N to W substitution, or N to Y substitution.

In embodiments, a mutation at position 515 includes an R to A substitution, R to C substitution, R to D substitution, R to E substitution, R to F substitution, R to G substitution, R to I substitution, R to K substitution, R to L substitution, R to M substitution, R to N substitution, R to P substitution, R to Q substitution, R to S substitution, R to T substitution, R to V substitution, R to W substitution, or R to Y substitution.

In embodiments, a mutation at position 517 includes an R to A substitution, R to C substitution, R to D substitution, R to E substitution, R to F substitution, R to G substitution, R to H substitution, R to I substitution, R to K substitution, R to L substitution, R to M substitution, R to N substitution, R to P substitution, R to Q substitution, R to S substitution, R to T substitution, R to V substitution, R to W substitution, or R to Y substitution.

In embodiments, the at least one mutation in the amino acid sequence's 513-loop is as shown in one of Table 1 to Table 9.

### Food Products Comprising S53 Family Proteases

S53 family proteases of the present disclosure can be used in the production of superior food products, i.e., by adding the proteases to a food and/or incorporated into a manufactured food for an animal's consumption or a human's consumption.

A food comprising a food protein that would benefit food protein's nutritional value and bioavailability may be used in a food product of the present disclosure. As mentioned above, the nutritional value of a food protein lies, in part, in its bioavailability following proteolytic digestion in an animal or human's digestive systems. Not all food proteins are sufficiently digested and/or resist proteolytic digesting in the gut, thereby limiting the food protein's nutritional value and bioavailability.

By combining a S53 family proteases of the present disclosure with a food comprising a food protein, the food protein can be more fully digested (before ingestion or after ingestion), thereby enhancing the food protein's nutritional value and bioavailability.

The food product may be an unprocessed plant or animal part (e.g., beans, peas, chicken parts, beef and the like) or may be a processed food product comprising or derived from one or more of the food proteins identified here. For example, the food products may comprise a plant or animal protein isolate or protein concentrate (e. g., soy protein, casein, or whey).

In an illustrative embodiment, the amount of protease included in a food product may typically comprise from about 0.001% (w/w) to about 1.0% (w/w) protease to food protein or food. In embodiments, the amount of protease included in a food product may comprise from 0.001% (w/w) to about 0.01% (w/w) protease to food protein or food; 0.01% (w/w) to about 0.10% (w/w) protease to food protein or food; or 0.1% (w/w) to about 1.0% (w/w) protease to food protein or food. In embodiments, the amount of protease included in a food product may comprise about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.20%, about 0.30%, about 0.40%, about 0.50%, about 0.60%, about 0.70%, about 0.80%, about 0.90%, or about 1.00% (w/w) protease to food protein.

In some embodiments, about 1 milligram (mg) to about 10 milligrams (mg) of the S53 protease of the present disclosure is combined with about 5 grams (g) to about 60 grams (g) of a food protein. In some embodiments, the provided amount of the S53 protease of the present disclosure comprises about 1 milligram (mg) to about 2 milligrams (mg), about 1 milligram (mg) to about 3 milligrams (mg), about 1 milligram (mg) to about 4 milligrams (mg), about 1 milligram (mg) to about 5 milligrams (mg), about 1 milligram (mg) to about 6 milligrams (mg), about 1 milligram (mg) to about 7 milligrams (mg), about 1 milligram (mg) to about 8 milligrams (mg), about 1 milligram (mg) to about 9 milligrams (mg), about 1 milligram (mg) to about 10 milligrams (mg), about 2 milligrams (mg) to about 3 milligrams (mg), about 2 milligrams (mg) to about 4 milligrams (mg), about 2 milligrams (mg) to about 5 milligrams (mg), about 2 milligrams (mg) to about 6 milligrams (mg), about 2 milligrams (mg) to about 7 milligrams (mg), about 2 milligrams (mg) to about 8 milligrams (mg), about 2 milligrams (mg) to about 9 milligrams (mg), about 2 milligrams (mg) to about 10 milligrams (mg), about 3 milligrams (mg) to about 4 milligrams (mg), about 3 milligrams (mg) to about 5 milligrams (mg), about 3 milligrams (mg) to about 6 milligrams (mg), about 3 milligrams (mg) to about 7 milligrams (mg), about 3 milligrams (mg) to about 8 milligrams (mg), about 3 milligrams (mg) to about 9 milligrams (mg), about 3 milligrams (mg) to about 10 milligrams (mg), about 4 milligrams (mg) to about 5 milligrams (mg), about 4 milligrams (mg) to about 6 milligrams (mg), about 4 milligrams (mg) to about 7 milligrams (mg), about 4 milligrams (mg) to about 8 milligrams (mg), about 4 milligrams (mg) to about 9 milligrams (mg), about 4 milligrams (mg) to about 10 milligrams (mg), about 5 milligrams (mg) to about 6 milligrams (mg), about 5 milligrams (mg) to about 7 milligrams (mg), about 5 milligrams (mg) to about 8 milligrams (mg), about 5 milligrams (mg) to about 9 milligrams (mg), about 5 milligrams (mg) to about 10 milligrams (mg), about 6 milligrams (mg) to about 7 milligrams (mg), about 6 milligrams (mg) to about 8 milligrams (mg), about 6 milligrams (mg) to about 9 milligrams (mg), about 6 milligrams (mg) to about 10 milligrams (mg), about 7 milligrams (mg) to about 8 milligrams (mg), about 7 milligrams (mg) to about 9 milligrams (mg), about 7 milligrams (mg) to about 10 milligrams (mg), about 8 milligrams (mg) to about 9 milligrams (mg), about 8 milligrams (mg) to about 10 milligrams (mg), or about 9 milligrams (mg) to about 10 milligrams (mg) and the amount of food protein comprises about 5 grams (g) to about 10 grams (g), about 5 grams (g) to about 15 grams (g), about 5 grams (g) to about 20 grams (g), about 5 grams (g) to about 25 grams (g), about 5 grams (g) to about 30 grams (g), about 5 grams (g) to about 35 grams (g), about 5 grams (g) to about 40 grams (g), about 5 grams (g) to about 45 grams (g), about 5 grams (g) to about 50 grams (g), about 5 grams (g) to about 60 grams (g), about 10 grams (g) to about 15 grams (g), about 10 grams (g) to about 20 grams (g), about 10 grams (g) to about 25 grams (g), about 10 grams (g) to about 30 grams (g), about 10 grams (g) to about 35 grams (g), about 10 grams (g) to about 40 grams (g), about 10 grams (g) to about 45 grams (g), about 10 grams (g) to about 50 grams (g), about 10 grams (g) to about 60 grams (g), about 15 grams (g) to about 20 grams (g), about 15 grams (g) to about 25 grams (g), about 15 grams (g) to about 30 grams (g), about 15 grams (g) to about 35 grams (g), about 15 grams (g) to about 40 grams (g), about 15 grams (g) to about 45 grams (g), about 15 grams (g) to about 50 grams (g), about 15 grams (g) to about 60 grams (g), about 20 grams (g) to about 25 grams (g), about 20 grams (g) to about 30 grams (g), about 20 grams (g) to about 35 grams (g), about 20 grams (g) to about 40 grams (g), about 20 grams (g) to about 45 grams (g), about 20 grams (g) to about 50 grams (g), about 20 grams (g) to about 60 grams (g), about 25 grams (g) to about 30 grams (g), about 25 grams (g) to about 35 grams (g), about 25 grams (g) to about 40 grams (g), about 25 grams (g) to about 45 grams (g), about 25 grams (g) to about 50 grams (g), about 25 grams (g) to about 60 grams (g), about 30 grams (g) to about 35 grams (g), about 30 grams (g) to about 40 grams (g), about 30 grams (g) to about 45 grams (g), about 30 grams (g) to about 50 grams (g), about 30 grams (g) to about 60 grams (g), about 35 grams (g) to about 40 grams (g), about 35 grams (g) to about 45 grams (g), about 35 grams (g) to about 50 grams (g), about 35 grams (g) to about 60 grams (g), about 40 grams (g) to about 45 grams (g), about 40 grams (g) to about 50 grams (g), about 40 grams (g) to about 60 grams (g), about 45 grams (g) to about 50 grams (g), about 45 grams (g) to about 60 grams (g), or about 50 grams (g) to about 60 grams (g).

In some embodiments, about 10 milligrams (mg) to about 100 milligrams (mg) of the S53 protease of the present disclosure is combined with about 5 grams (g) to about 60 grams (g) of a food protein. In some embodiments, the provided amount of the S53 protease of the present disclosure comprises about 10 milligram (mg) to about 20 milligrams (mg), about 10 milligram (mg) to about 30 milligrams (mg), about 10 milligram (mg) to about 40 milligrams (mg), about 10 milligram (mg) to about 50 milligrams (mg), about 10 milligram (mg) to about 60 milligrams (mg), about 10 milligram (mg) to about 70 milligrams (mg), about 10 milligram (mg) to about 80 milligrams (mg), about 10 milligram (mg) to about 90 milligrams (mg), about 10 milligram (mg) to about 100 milligrams (mg), about 20 milligrams (mg) to about 30 milligrams (mg), about 20 milligrams (mg) to about 40 milligrams (mg), about 20 milligrams (mg) to about 50 milligrams (mg), about 20 milligrams (mg) to about 60 milligrams (mg), about 20 milligrams (mg) to about 70 milligrams (mg), about 20 milligrams (mg) to about 80 milligrams (mg), about 20 milligrams (mg) to about 90 milligrams (mg), about 20 milligrams (mg) to about 100 milligrams (mg), about 30 milligrams (mg) to about 40 milligrams (mg), about 30 milligrams (mg) to about 50 milligrams (mg), about 30 milligrams (mg) to about 60 milligrams (mg), about 30 milligrams (mg) to about 70 milligrams (mg), about 30 milligrams (mg) to about 80 milligrams (mg), about 30 milligrams (mg) to about 90 milligrams (mg), about 30 milligrams (mg) to about 100 milligrams (mg), about 40 milligrams (mg) to about 50 milligrams (mg), about 40 milligrams (mg) to about 60 milligrams (mg), about 40 milligrams (mg) to about 70 milligrams (mg), about 40 milligrams (mg) to about 80 milligrams (mg), about 40 milligrams (mg) to about 90 milligrams (mg), about 40 milligrams (mg) to about 100 milligrams (mg), about 50 milligrams (mg) to about 60 milligrams (mg), about 50 milligrams (mg) to about 70 milligrams (mg), about 50 milligrams (mg) to about 80 milligrams (mg), about 50 milligrams (mg) to about 90 milligrams (mg), about 50 milligrams (mg) to about 100 milligrams (mg), about 60 milligrams (mg) to about 70 milligrams (mg), about 60 milligrams (mg) to about 80 milligrams (mg), about 60 milligrams (mg) to about 90 milligrams (mg), about 60 milligrams (mg) to about 100 milligrams (mg), about 70 milligrams (mg) to about 80 milligrams (mg), about 70 milligrams (mg) to about 90 milligrams (mg), about 70 milligrams (mg) to about 100 milligrams (mg), about 80 milligrams (mg) to about 90 milligrams (mg), about 80 milligrams (mg) to about 100 milligrams (mg), or about 90 milligrams (mg) to about 100 milligrams (mg) and the amount of food protein comprises about 5 grams (g) to about 10 grams (g), about 5 grams (g) to about 15 grams (g), about 5 grams (g) to about 20 grams (g), about 5 grams (g) to about 25 grams (g), about 5 grams (g) to about 30 grams (g), about 5 grams (g) to about 35 grams (g), about 5 grams (g) to about 40 grams (g), about 5 grams (g) to about 45 grams (g), about 5 grams (g) to about 50 grams (g), about 5 grams (g) to about 60 grams (g), about 10 grams (g) to about 15 grams (g), about 10 grams (g) to about 20 grams (g), about 10 grams (g) to about 25 grams (g), about 10 grams (g) to about 30 grams (g), about 10 grams (g) to about 35 grams (g), about 10 grams (g) to about 40 grams (g), about 10 grams (g) to about 45 grams (g), about 10 grams (g) to about 50 grams (g), about 10 grams (g) to about 60 grams (g), about 15 grams (g) to about 20 grams (g), about 15 grams (g) to about 25 grams (g), about 15 grams (g) to about 30 grams (g), about 15 grams (g) to about 35 grams (g), about 15 grams (g) to about 40 grams (g), about 15 grams (g) to about 45 grams (g), about 15 grams (g) to about 50 grams (g), about 15 grams (g) to about 60 grams (g), about 20 grams (g) to about 25 grams (g), about 20 grams (g) to about 30 grams (g), about 20 grams (g) to about 35 grams (g), about 20 grams (g) to about 40 grams (g), about 20 grams (g) to about 45 grams (g), about 20 grams (g) to about 50 grams (g), about 20 grams (g) to about 60 grams (g), about 25 grams (g) to about 30 grams (g), about 25 grams (g) to about 35 grams (g), about 25 grams (g) to about 40 grams (g), about 25 grams (g) to about 45 grams (g), about 25 grams (g) to about 50 grams (g), about 25 grams (g) to about 60 grams (g), about 30 grams (g) to about 35 grams (g), about 30 grams (g) to about 40 grams (g), about 30 grams (g) to about 45 grams (g), about 30 grams (g) to about 50 grams (g), about 30 grams (g) to about 60 grams (g), about 35 grams (g) to about 40 grams (g), about 35 grams (g) to about 45 grams (g), about 35 grams (g) to about 50 grams (g), about 35 grams (g) to about 60 grams (g), about 40 grams (g) to about 45 grams (g), about 40 grams (g) to about 50 grams (g), about 40 grams (g) to about 60 grams (g), about 45 grams (g) to about 50 grams (g), about 45 grams (g) to about 60 grams (g), or about 50 grams (g) to about 60 grams (g).

In some embodiments, about 50 milligrams (mg) to about 1,500 milligrams (mg) of the S53 protease of the present disclosure is combined with about 5 grams (g) to about 60 grams (g) of a food protein.

In some embodiments, the provided amount of the S53 protease of the present disclosure comprises about 50 milligrams (mg) to about 100 milligrams (mg), about 50 milligrams (mg) to about 150 milligrams (mg), about 50 milligrams (mg) to about 200 milligrams (mg), about 50 milligrams (mg) to about 300 milligrams (mg), about 50 milligrams (mg) to about 400 milligrams (mg), about 50 milligrams (mg) to about 500 milligrams (mg), about 50 milligrams (mg) to about 750 milligrams (mg), about 50 milligrams (mg) to about 1,000 milligrams (mg), about 50 milligrams (mg) to about 1,500 milligrams (mg), about 100 milligrams (mg) to about 150 milligrams (mg), about 100 milligrams (mg) to about 200 milligrams (mg), about 100 milligrams (mg) to about 300 milligrams (mg), about 100 milligrams (mg) to about 400 milligrams (mg), about 100 milligrams (mg) to about 500 milligrams (mg), about 100 milligrams (mg) to about 750 milligrams (mg), about 100 milligrams (mg) to about 1,000 milligrams (mg), about 100 milligrams (mg) to about 1,500 milligrams (mg), about 150 milligrams (mg) to about 200 milligrams (mg), about 150 milligrams (mg) to about 300 milligrams (mg), about 150 milligrams (mg) to about 400 milligrams (mg), about 150 milligrams (mg) to about 500 milligrams (mg), about 150 milligrams (mg) to about 750 milligrams (mg), about 150 milligrams (mg) to about 1,000 milligrams (mg), about 150 milligrams (mg) to about 1,500 milligrams (mg), about 200 milligrams (mg) to about 300 milligrams (mg), about 200 milligrams (mg) to about 400 milligrams (mg), about 200 milligrams (mg) to about 500 milligrams (mg), about 200 milligrams (mg) to about 750 milligrams (mg), about 200 milligrams (mg) to about 1,000 milligrams (mg), about 200 milligrams (mg) to about 1,500 milligrams (mg), about 300 milligrams (mg) to about 400 milligrams (mg), about 300 milligrams (mg) to about 500 milligrams (mg), about 300 milligrams (mg) to about 750 milligrams (mg), about 300 milligrams (mg) to about 1,000 milligrams (mg), about 300 milligrams (mg) to about 1,500 milligrams (mg), about 400 milligrams (mg) to about 500 milligrams (mg), about 400 milligrams (mg) to about 750 milligrams (mg), about 400 milligrams (mg) to about 1,000 milligrams (mg), about 400 milligrams (mg) to about 1,500 milligrams (mg), about 500 milligrams (mg) to about 750 milligrams (mg), about 500 milligrams (mg) to about 1,000 milligrams (mg), about 500 milligrams (mg) to about 1,500 milligrams (mg), about 750 milligrams (mg) to about 1,000 milligrams (mg), about 750 milligrams (mg) to about 1,500 milligrams (mg), or about 1,000 milligrams (mg) to about 1,500 milligrams (mg) and the amount of food protein comprises about 5 grams (g) to about 10 grams (g), about 5 grams (g) to about 15 grams (g), about 5 grams (g) to about 20 grams (g), about 5 grams (g) to about 25 grams (g), about 5 grams (g) to about 30 grams (g), about 5 grams (g) to about 35 grams (g), about 5 grams (g) to about 40 grams (g), about 5 grams (g) to about 45 grams (g), about 5 grams (g) to about 50 grams (g), about 5 grams (g) to about 60 grams (g), about 10 grams (g) to about 15 grams (g), about 10 grams (g) to about 20 grams (g), about 10 grams (g) to about 25 grams (g), about 10 grams (g) to about 30 grams (g), about 10 grams (g) to about 35 grams (g), about 10 grams (g) to about 40 grams (g), about 10 grams (g) to about 45 grams (g), about 10 grams (g) to about 50 grams (g), about 10 grams (g) to about 60 grams (g), about 15 grams (g) to about 20 grams (g), about 15 grams (g) to about 25 grams (g), about 15 grams (g) to about 30 grams (g), about 15 grams (g) to about 35 grams (g), about 15 grams (g) to about 40 grams (g), about 15 grams (g) to about 45 grams (g), about 15 grams (g) to about 50 grams (g), about 15 grams (g) to about 60 grams (g), about 20 grams (g) to about 25 grams (g), about 20 grams (g) to about 30 grams (g), about 20 grams (g) to about 35 grams (g), about 20 grams (g) to about 40 grams (g), about 20 grams (g) to about 45 grams (g), about 20 grams (g) to about 50 grams (g), about 20 grams (g) to about 60 grams (g), about 25 grams (g) to about 30 grams (g), about 25 grams (g) to about 35 grams (g), about 25 grams (g) to about 40 grams (g), about 25 grams (g) to about 45 grams (g), about 25 grams (g) to about 50 grams (g), about 25 grams (g) to about 60 grams (g), about 30 grams (g) to about 35 grams (g), about 30 grams (g) to about 40 grams (g), about 30 grams (g) to about 45 grams (g), about 30 grams (g) to about 50 grams (g), about 30 grams (g) to about 60 grams (g), about 35 grams (g) to about 40 grams (g), about 35 grams (g) to about 45 grams (g), about 35 grams (g) to about 50 grams (g), about 35 grams (g) to about 60 grams (g), about 40 grams (g) to about 45 grams (g), about 40 grams (g) to about 50 grams (g), about 40 grams (g) to about 60 grams (g), about 45 grams (g) to about 50 grams (g), about 45 grams (g) to about 60 grams (g), or about 50 grams (g) to about 60 grams (g).

In some embodiments, about 50 milligrams (mg), about 100 milligrams (mg), about 150 milligrams (mg), about 200 milligrams (mg), about 300 milligrams (mg), about 400 milligrams (mg), about 500 milligrams (mg), about 750 milligrams (mg), about 1,000 milligrams (mg), or about 1,500 milligrams (mg) of the S53 protease of the present disclosure is combined with about 5 grams (g), about 10 grams (g), about 15 grams (g), about 20 grams (g), about 25 grams (g), about 30 grams (g), about 35 grams (g), about 40 grams (g), about 45 grams (g), about 50 grams (g), or about 60 grams (g) of the food protein.

In some embodiments, at least about 50 milligrams (mg), about 100 milligrams (mg), about 150 milligrams (mg), about 200 milligrams (mg), about 300 milligrams (mg), about 400 milligrams (mg), about 500 milligrams (mg), about 750 milligrams (mg), or about 1,000 milligrams (mg) of the S53 protease of the present disclosure is combined with at least about 5 grams (g), about 10 grams (g), about 15 grams (g), about 20 grams (g), about 25 grams (g), about 30 grams (g), about 35 grams (g), about 40 grams (g), about 45 grams (g), or about 50 grams (g) of the food protein.

In some embodiments, at most about 100 milligrams (mg), about 150 milligrams (mg), about 200 milligrams (mg), about 300 milligrams (mg), about 400 milligrams (mg), about 500 milligrams (mg), about 750 milligrams (mg), about 1,000 milligrams (mg), or about 1,500 milligrams (mg) of the S53 protease of the present disclosure is combined with at most about 10 grams (g), about 15 grams (g), about 20 grams (g), about 25 grams (g), about 30 grams (g), about 35 grams (g), about 40 grams (g), about 45 grams (g), about 50 grams (g), or about 60 grams (g) of the food protein.

One of skill will appreciate that the food products of the present disclosure can comprise more than one of the herein-disclosed proteases. For example, a food product may comprise one, two, three, four, or more herein-disclosed proteases.

An aspect of the present disclosure provides a food product including an S53 family protease having an amino acid sequence that is at least 80% identical to SEQ ID NO: 1 and includes at least one mutation in the amino acid sequence's 513-loop which corresponds to amino acids 513 to 517 of SEQ ID NO: 1 and a protein obtained from or naturally present in Adzuki beans, Almonds, Amaranth, Asparagus, Baby Lima, Barley, Beef, Black beans, Black eyed peas, Broccoli, Buckwheat, Cannellini beans, Cashews, Chia seeds, Chicken, Chickpea, Chlorella, Corn, Cowpea, Cranberry beans, Crowder pea, Egg, e.g., chicken egg, Fava beans, Field Peas, Flounder, Great Northern Beans, Green beans, Hemp protein powder, Kamut, Kidney beans, Lady cream peas, Lentil, Lupine Beans, Masoor Dal (Indian Red lentils), Milk or other milk products, e.g., cheese and yoghurt, Mung beans, Navy pea, Pea, Peanut, Pigeon Peas, Pink beans, Pinto beans, Pistachios, Pork, Quinoa, Royal Canin, Rye berries, Salmon, Soy, Spirulina, Sunflower seeds, Turkey, White beans, and Yellow split peas.

In embodiments, the at least one mutation provides increased thermostability, increased enzymatic activity, and/or increased substrate specificity, relative to an S53 family protease having an amino acid sequence that lacks the at least one mutation located at position 513, 514, 515, and/or 517 with respect to SEQ ID NO: 1.

In embodiments, the S53 family protease includes at least two mutations in the 513-loop relative to SEQ ID NO: 1. In some cases, the at least two mutations are located at positions 513 and 514; at positions 513 and 515; at positions 513 and 517; at positions 514 and 515; at positions 514 and 517; or at positions 515 and 517.

In embodiments, the S53 family protease includes at least three mutations in the 513-loop relative to SEQ ID NO: 1. In some cases, the at least three mutations are located at positions 513, 515, and 517; at positions 513, 514, and 517; at positions 513, 514, and 515; or at positions 514, 515, and 517.

In embodiments, the S53 family protease includes at least four mutations in the 513-loop relative to SEQ ID NO: 1.

In embodiments, the S53 family protease includes five mutations in the 513-loop relative to SEQ ID NO: 1 which provide increased enzymatic activity and/or increased substrate specificity.

In embodiments, a mutation at position 513 includes an A to C substitution, A to D substitution, A to E substitution, A to F substitution, A to G substitution, A to H substitution, A to I substitution, A to K substitution, A to L substitution, A to M substitution, A to N substitution, A to P substitution, A to Q substitution, A to R substitution, A to S substitution, A to T substitution, A to V substitution, A to W substitution, or A to Y substitution.

In embodiments, a mutation at position 514 includes an N to A substitution, N to C substitution, N to D substitution, N to E substitution, N to F substitution, N to G substitution, N to H substitution, N to I substitution, N to K substitution, N to L substitution, N to M substitution, N to Q substitution, N to R substitution, N to S substitution, N to T substitution, N to V substitution, N to W substitution, or N to Y substitution.

In embodiments, a mutation at position 515 includes an R to A substitution, R to C substitution, R to D substitution, R to E substitution, R to F substitution, R to G substitution, R to I substitution, R to K substitution, R to L substitution, R to M substitution, R to N substitution, R to P substitution, R to Q substitution, R to S substitution, R to T substitution, R to V substitution, R to W substitution, or R to Y substitution.

In embodiments, a mutation at position 517 includes an R to A substitution, R to C substitution, R to D substitution, R to E substitution, R to F substitution, R to G substitution, R to H substitution, R to I substitution, R to K substitution, R to L substitution, R to M substitution, R to N substitution, R to P substitution, R to Q substitution, R to S substitution, R to T substitution, R to V substitution, R to W substitution, or R to Y substitution.

In embodiments, the at least one mutation in the amino acid sequence's 513-loop is as shown in one of Table 1 to Table 9.

In embodiments, the S53 family protease further includes one or more additional mutations at position 13, 15, 18, 23, 35, 38, 40, 51, 52, 55, 64, 70, 73, 91, 96, 101, 121, 123, 143, 145, 147, 155, 174, 175, 181, 188, 198, 218, 228, 229, 236, 240, 243, 253, 261, 265, 268, 273, 280, 283, 286, 290, 292, 296, 297, 300, 303, 308, 312, 314, 319, 323, 324, 325, 326, 327, 328, 329, 330, 332, 341, 353, 359, 360, 371, 377, 385, 387, 390, 391, 392, 398, 404, 411, 417, 421, 422, 432, 433, 434, 441, 446, 448, 452, 459, 469, 474, 486, 499, 500, 501, 509, 513, 514, 515, 516, 517, 537, and/or 550 relative to SEQ ID NO: 1. In some cases, the one or more additional mutations includes substitutions selected from K13A, E15A, A18Q, R23A, T35A, Q38E, 140M, G51A, D52G, E55A, L641, V70E, E73K, 191 L, E96D, T101A, V121I, Q123R, V143L, D145E, R147T, L155M, L174M, R175Q, E181G, S188A, T228A, S236S, S240P, T253S, N261S, I283F, N297D, V303I, H308L, I312V, A325T, P326S, S328A, A387G, E391A, R392Q, S404A, S417A, R421H, G433S, V441 L, T459A, P486A, P499A, E500D, R517Q, 1537V, and/or A550P relative to SEQ ID NO: 1. In some cases, the one or more additional mutations includes substitutions selected from A325K; A325L; P326F; P326L; P326W; S328Q; S328H; E391V; E391L; E391I; E391M; S417W; V441W; T459W; T459R; P486Q; and/or R517G relative to SEQ ID NO: 1.

In embodiments, the present disclosure relates to an S53 family protease, further including one or more substrate selectivity mutations wherein the one or more substrate selectivity mutations increase protease substrate selectivity relative to a protease including an amino acid sequence as shown in SEQ ID NO: 1 and wherein the one or more additional mutations is at position 198, 218, 229, 243, 265, 268, 273, 280, 286, 290, 292, 296, 300, 314, 319, 323, 324, 325, 326, 327, 328, 329, 330, 332, 341, 353, 359, 360, 371, 377, 385, 390, 391, 398, 411, 417, 422, 432, 433, 434, 441, 446, 448, 452, 459, 469, 474, 486, 501, 509, 513, 514, 515, 516, and/or 517 relative to SEQ ID NO: 1.

In embodiments, the one or more substrate selectivity mutations includes substitutions selected from D198E, I218L, S229D, Q243G, G265A, E268M, E268T, E268C, E268H, E268Q, V273I, G280R, Y286K, N290T, N290S, D292F, D292Y, L296T, T300S, S314P, G319Q, S323R, W324K, A325K, A325L, P326F, P326W, P326L, A327Y, A327D, S328H, S328Q, I329L, A330F, M332I, M332L, A341 R, S353E, E359V, E359L, Q360C, A371S, A377G, A385V, A385L, I390W, E391V, E391M, E391 L, E391I, D398S, R411Q, R411M, R411E, R411D, S417W, A422V, A432G, G433G, S434I, S434R, S434T, S434V, V441W, D446S, A448N, E452Y, E452W, E452F, T459R, T459W, A469V, A474V, P486Q, V501C, N509H, A513T, A513D, A513Y, N514G, R515L, R515E, R515I, A516T, A516S, and/or R517G relative to SEQ ID NO: 1. In some cases, the S53 family protease further includes one or more substrate selectivity mutations wherein the one or more substrate selectivity mutations increase protease substrate selectivity when casein is a substrate relative to a protease including an amino acid sequence as shown in SEQ ID NO: 1 and wherein the one or more additional mutations is at position 198, 218, 229, 243, 265, 268, 273, 280, 286, 290, 292, 296, 300, 314, 319, 323, 324, 325, 326, 327, 328, 329, 330, 332, 341, 353, 359, 360, 371, 377, 385, 390, 391, 398, 411, 417, 422, 432, 433, 434, 441, 446, 448, 452, 459, 469, 474, 486, 501, 509, 513, 514, 515, 516, and/or 517 relative to SEQ ID NO: 1. In some cases, the one or more substrate selectivity mutations includes substitutions selected from D198E, I218L, S229D, Q243G, G265A, E268M, E268T, E268C, E268H, E268Q, V273I, G280R, Y286K, N290T, N290S, D292F, D292Y, L296T, T300S, S314P, G319Q, S323R, W324K, A325K, A325L, P326F, P326W, P326L, A327D, A327Y, S328Q, S328H, I329L, A330F, M332L, M332I, A341 R, S353E, E359L, E359V, Q360C, A371S, A377G, A385L, A385V, I390W, E391V, E391L, E391I, E391M, D398S, R411D, R411E, R411M, R411Q, S417W, A422V, A432G, G433G, S434I, S434R, S434T, S434V, V441W, D446S, A448N, A448Y, E452Y, E452W, E452F, T459R, T459W, A469V, A474V, P486Q, V501C, N509H, A513Y, A513D, A513T, N514G, R515I, R515E, R515L, A516S, A516T, and/or R517G relative to SEQ ID NO: 1.

In embodiments, the S53 family protease further includes one or more substrate selectivity mutations wherein the one or more substrate selectivity mutations increase protease substrate selectivity when zein is a substrate relative to a protease including an amino acid sequence as shown in SEQ ID NO: 1 and wherein the one or more additional mutations is at position 198, 218, 229, 243, 265, 268, 273, 280, 286, 290, 292, 296, 300, 314, 319, 323, 324, 325, 326, 327, 328, 329, 330, 332, 341, 353, 359, 360, 371, 377, 385, 390, 391, 398, 411, 417, 422, 432, 433, 434, 441, 446, 448, 452, 459, 469, 474, 486, 501, 509, 513, 514, 515, 516, and/or 517 relative to SEQ ID NO: 1. In some cases, the one or more substrate selectivity mutations includes substitutions selected from D198E, I218L, S229D, Q243G, G265A, E268M, E268T, E268C, E268H, E268Q, V273I, G280R, Y286K, N290T, N290S, D292Y, D292F, L296T, T300S, S314P, G319Q, S323R, W324K, A325K, A325L, P326F, P326L, P326W, A327D, A327Y, S328Q, S328H, I329L, A330F, M332I, M332L, A341 R, S353E, E359L, E359V, Q360C, A371S, A377G, A385L, A385V, I390W, E391V, E391M, E391I, E391L, D398S, R411Q, R411M, R411E, R411D, S417W, A422V, A432G, G433G, S434I, S434R, S434T, S434V, V441W, D446S, A448N, E452F, E452W, E452F, T459R, T459W, A469V, A474V, P486Q, V501 C, N509H, A513T, A513D, A513Y, N514G, R515I, R515E, R515L, A516S, A516T, and/or R517G relative to SEQ ID NO: 1.

In embodiments, the amino acid sequence lacks a mutation at position 198, 218, 229, 243, 265, 268, 273, 280, 286, 290, 292, 296, 300, 314, 319, 323, 324, 325, 326, 327, 328, 329, 330, 332, 341, 353, 359, 360, 371, 377, 385, 390, 391, 398, 411, 417, 422, 432, 433, 434, 441, 446, 448, 452, 459, 469, 474, 486, 501, and/or 509 relative to SEQ ID NO: 1.

In embodiments, the amino acid sequence lacks a mutation at position 266, 270, 352, and/or 466 relative to SEQ ID NO: 1.

In embodiments, the amino acid sequence lacks a mutation at each of positions 266, 270, 352, and 466 relative to SEQ ID NO: 1.

In embodiments, the amino acid sequence lacks a mutation position 516 relative to an amino acid SEQ ID NO: 1.

In embodiments, the amino acid sequence includes a mutation position 516 relative to an amino acid SEQ ID NO: 1. In some cases, the mutation at position 516 includes T.

In embodiments, the amino acid sequence is at least 85% identical to SEQ ID NO: 1, is at least 90% identical to SEQ ID NO: 1, is at least 95% identical to SEQ ID NO: 1, is at least 96% identical to SEQ ID NO: 1, is at least 97% identical to SEQ ID NO: 1, is at least 98% identical to SEQ ID NO: 1, or is at least 99% identical to SEQ ID NO: 1 and maintains or increases thermostability, enzymatic activity, and/or substrate specificity relative to an S53 family protease having the amino acid sequence of SEQ ID NO: 1.

### Food Supplements Comprising S53 Family Proteases

S53 family proteases of the present disclosure can be used in the manufacture of food supplements, e.g., dietary supplements, nutritional supplements, sports nutrition supplements, digestive aid supplements, and the like.

The food supplements may be in various dosage forms, including for example, tablet, capsule, powder, granule, pellet, soft gel, hard gel, controlled release form, liquid, syrup, suspension, emulsion, and the like. Any commercially acceptable formulation known to be suitable for use in food supplements may be used in the food supplements of the present disclosure.

As noted above, the present disclosure is based, at least in part, on the discovery of S53 family proteases that have increased thermostability, increased enzymatic activity, and/or increased substrate specificity in digesting target food proteins. The food supplement may be designed to be ingested with the food comprising the target food protein or may be ingested just before or just after the food, typically within two hours before or after ingesting the food. Thus, for the purposes of the present disclosure, a protease of the present disclosure, or a food supplement comprising the protease, is “ingested with” a food, if it is ingested simultaneously with the food or within two hours before or after ingestion of the food.

In an illustrative embodiment, a unit dose of a food supplement of the disclosure will typically comprise from about 0.001% (w/w) to about 1.0% (w/w) protease to food protein. In embodiments, the amount of protease included in a food product may comprise from 0.001% (w/w) to about 0.01% (w/w) protease to food protein or food; 0.01% (w/w) to about 0.10% (w/w) protease to food protein or food; or 0.1% (w/w) to about 1.0% (w/w) protease to food protein or food. In embodiments, the amount of protease included in a food product may comprise about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.20%, about 0.30%, about 0.40%, about 0.50%, about 0.60%, about 0.70%, about 0.80%, about 0.90%, or about 1.00% (w/w) protease to food protein.

In some embodiments, about 1 milligram (mg) to about 10 milligrams (mg) of the S53 protease of the present disclosure is combined with about 5 grams (g) to about 60 grams (g) of a food protein. In some embodiments, the provided amount of the S53 protease of the present disclosure comprises about 1 milligram (mg) to about 2 milligrams (mg), about 1 milligram (mg) to about 3 milligrams (mg), about 1 milligram (mg) to about 4 milligrams (mg), about 1 milligram (mg) to about 5 milligrams (mg), about 1 milligram (mg) to about 6 milligrams (mg), about 1 milligram (mg) to about 7 milligrams (mg), about 1 milligram (mg) to about 8 milligrams (mg), about 1 milligram (mg) to about 9 milligrams (mg), about 1 milligram (mg) to about 10 milligrams (mg), about 2 milligrams (mg) to about 3 milligrams (mg), about 2 milligrams (mg) to about 4 milligrams (mg), about 2 milligrams (mg) to about 5 milligrams (mg), about 2 milligrams (mg) to about 6 milligrams (mg), about 2 milligrams (mg) to about 7 milligrams (mg), about 2 milligrams (mg) to about 8 milligrams (mg), about 2 milligrams (mg) to about 9 milligrams (mg), about 2 milligrams (mg) to about 10 milligrams (mg), about 3 milligrams (mg) to about 4 milligrams (mg), about 3 milligrams (mg) to about 5 milligrams (mg), about 3 milligrams (mg) to about 6 milligrams (mg), about 3 milligrams (mg) to about 7 milligrams (mg), about 3 milligrams (mg) to about 8 milligrams (mg), about 3 milligrams (mg) to about 9 milligrams (mg), about 3 milligrams (mg) to about 10 milligrams (mg), about 4 milligrams (mg) to about 5 milligrams (mg), about 4 milligrams (mg) to about 6 milligrams (mg), about 4 milligrams (mg) to about 7 milligrams (mg), about 4 milligrams (mg) to about 8 milligrams (mg), about 4 milligrams (mg) to about 9 milligrams (mg), about 4 milligrams (mg) to about 10 milligrams (mg), about 5 milligrams (mg) to about 6 milligrams (mg), about 5 milligrams (mg) to about 7 milligrams (mg), about 5 milligrams (mg) to about 8 milligrams (mg), about 5 milligrams (mg) to about 9 milligrams (mg), about 5 milligrams (mg) to about 10 milligrams (mg), about 6 milligrams (mg) to about 7 milligrams (mg), about 6 milligrams (mg) to about 8 milligrams (mg), about 6 milligrams (mg) to about 9 milligrams (mg), about 6 milligrams (mg) to about 10 milligrams (mg), about 7 milligrams (mg) to about 8 milligrams (mg), about 7 milligrams (mg) to about 9 milligrams (mg), about 7 milligrams (mg) to about 10 milligrams (mg), about 8 milligrams (mg) to about 9 milligrams (mg), about 8 milligrams (mg) to about 10 milligrams (mg), or about 9 milligrams (mg) to about 10 milligrams (mg) and the amount of food protein comprises about 5 grams (g) to about 10 grams (g), about 5 grams (g) to about 15 grams (g), about 5 grams (g) to about 20 grams (g), about 5 grams (g) to about 25 grams (g), about 5 grams (g) to about 30 grams (g), about 5 grams (g) to about 35 grams (g), about 5 grams (g) to about 40 grams (g), about 5 grams (g) to about 45 grams (g), about 5 grams (g) to about 50 grams (g), about 5 grams (g) to about 60 grams (g), about 10 grams (g) to about 15 grams (g), about 10 grams (g) to about 20 grams (g), about 10 grams (g) to about 25 grams (g), about 10 grams (g) to about 30 grams (g), about 10 grams (g) to about 35 grams (g), about 10 grams (g) to about 40 grams (g), about 10 grams (g) to about 45 grams (g), about 10 grams (g) to about 50 grams (g), about 10 grams (g) to about 60 grams (g), about 15 grams (g) to about 20 grams (g), about 15 grams (g) to about 25 grams (g), about 15 grams (g) to about 30 grams (g), about 15 grams (g) to about 35 grams (g), about 15 grams (g) to about 40 grams (g), about 15 grams (g) to about 45 grams (g), about 15 grams (g) to about 50 grams (g), about 15 grams (g) to about 60 grams (g), about 20 grams (g) to about 25 grams (g), about 20 grams (g) to about 30 grams (g), about 20 grams (g) to about 35 grams (g), about 20 grams (g) to about 40 grams (g), about 20 grams (g) to about 45 grams (g), about 20 grams (g) to about 50 grams (g), about 20 grams (g) to about 60 grams (g), about 25 grams (g) to about 30 grams (g), about 25 grams (g) to about 35 grams (g), about 25 grams (g) to about 40 grams (g), about 25 grams (g) to about 45 grams (g), about 25 grams (g) to about 50 grams (g), about 25 grams (g) to about 60 grams (g), about 30 grams (g) to about 35 grams (g), about 30 grams (g) to about 40 grams (g), about 30 grams (g) to about 45 grams (g), about 30 grams (g) to about 50 grams (g), about 30 grams (g) to about 60 grams (g), about 35 grams (g) to about 40 grams (g), about 35 grams (g) to about 45 grams (g), about 35 grams (g) to about 50 grams (g), about 35 grams (g) to about 60 grams (g), about 40 grams (g) to about 45 grams (g), about 40 grams (g) to about 50 grams (g), about 40 grams (g) to about 60 grams (g), about 45 grams (g) to about 50 grams (g), about 45 grams (g) to about 60 grams (g), or about 50 grams (g) to about 60 grams (g).

In some embodiments, about 10 milligrams (mg) to about 100 milligrams (mg) of the S53 protease of the present disclosure is combined with about 5 grams (g) to about 60 grams (g) of a food protein. In some embodiments, the provided amount of the S53 protease of the present disclosure comprises about 10 milligram (mg) to about 20 milligrams (mg), about 10 milligram (mg) to about 30 milligrams (mg), about 10 milligram (mg) to about 40 milligrams (mg), about 10 milligram (mg) to about 50 milligrams (mg), about 10 milligram (mg) to about 60 milligrams (mg), about 10 milligram (mg) to about 70 milligrams (mg), about 10 milligram (mg) to about 80 milligrams (mg), about 10 milligram (mg) to about 90 milligrams (mg), about 10 milligram (mg) to about 100 milligrams (mg), about 20 milligrams (mg) to about 30 milligrams (mg), about 20 milligrams (mg) to about 40 milligrams (mg), about 20 milligrams (mg) to about 50 milligrams (mg), about 20 milligrams (mg) to about 60 milligrams (mg), about 20 milligrams (mg) to about 70 milligrams (mg), about 20 milligrams (mg) to about 80 milligrams (mg), about 20 milligrams (mg) to about 90 milligrams (mg), about 20 milligrams (mg) to about 100 milligrams (mg), about 30 milligrams (mg) to about 40 milligrams (mg), about 30 milligrams (mg) to about 50 milligrams (mg), about 30 milligrams (mg) to about 60 milligrams (mg), about 30 milligrams (mg) to about 70 milligrams (mg), about 30 milligrams (mg) to about 80 milligrams (mg), about 30 milligrams (mg) to about 90 milligrams (mg), about 30 milligrams (mg) to about 100 milligrams (mg), about 40 milligrams (mg) to about 50 milligrams (mg), about 40 milligrams (mg) to about 60 milligrams (mg), about 40 milligrams (mg) to about 70 milligrams (mg), about 40 milligrams (mg) to about 80 milligrams (mg), about 40 milligrams (mg) to about 90 milligrams (mg), about 40 milligrams (mg) to about 100 milligrams (mg), about 50 milligrams (mg) to about 60 milligrams (mg), about 50 milligrams (mg) to about 70 milligrams (mg), about 50 milligrams (mg) to about 80 milligrams (mg), about 50 milligrams (mg) to about 90 milligrams (mg), about 50 milligrams (mg) to about 100 milligrams (mg), about 60 milligrams (mg) to about 70 milligrams (mg), about 60 milligrams (mg) to about 80 milligrams (mg), about 60 milligrams (mg) to about 90 milligrams (mg), about 60 milligrams (mg) to about 100 milligrams (mg), about 70 milligrams (mg) to about 80 milligrams (mg), about 70 milligrams (mg) to about 90 milligrams (mg), about 70 milligrams (mg) to about 100 milligrams (mg), about 80 milligrams (mg) to about 90 milligrams (mg), about 80 milligrams (mg) to about 100 milligrams (mg), or about 90 milligrams (mg) to about 100 milligrams (mg) and the amount of food protein comprises about 5 grams (g) to about 10 grams (g), about 5 grams (g) to about 15 grams (g), about 5 grams (g) to about 20 grams (g), about 5 grams (g) to about 25 grams (g), about 5 grams (g) to about 30 grams (g), about 5 grams (g) to about 35 grams (g), about 5 grams (g) to about 40 grams (g), about 5 grams (g) to about 45 grams (g), about 5 grams (g) to about 50 grams (g), about 5 grams (g) to about 60 grams (g), about 10 grams (g) to about 15 grams (g), about 10 grams (g) to about 20 grams (g), about 10 grams (g) to about 25 grams (g), about 10 grams (g) to about 30 grams (g), about 10 grams (g) to about 35 grams (g), about 10 grams (g) to about 40 grams (g), about 10 grams (g) to about 45 grams (g), about 10 grams (g) to about 50 grams (g), about 10 grams (g) to about 60 grams (g), about 15 grams (g) to about 20 grams (g), about 15 grams (g) to about 25 grams (g), about 15 grams (g) to about 30 grams (g), about 15 grams (g) to about 35 grams (g), about 15 grams (g) to about 40 grams (g), about 15 grams (g) to about 45 grams (g), about 15 grams (g) to about 50 grams (g), about 15 grams (g) to about 60 grams (g), about 20 grams (g) to about 25 grams (g), about 20 grams (g) to about 30 grams (g), about 20 grams (g) to about 35 grams (g), about 20 grams (g) to about 40 grams (g), about 20 grams (g) to about 45 grams (g), about 20 grams (g) to about 50 grams (g), about 20 grams (g) to about 60 grams (g), about 25 grams (g) to about 30 grams (g), about 25 grams (g) to about 35 grams (g), about 25 grams (g) to about 40 grams (g), about 25 grams (g) to about 45 grams (g), about 25 grams (g) to about 50 grams (g), about 25 grams (g) to about 60 grams (g), about 30 grams (g) to about 35 grams (g), about 30 grams (g) to about 40 grams (g), about 30 grams (g) to about 45 grams (g), about 30 grams (g) to about 50 grams (g), about 30 grams (g) to about 60 grams (g), about 35 grams (g) to about 40 grams (g), about 35 grams (g) to about 45 grams (g), about 35 grams (g) to about 50 grams (g), about 35 grams (g) to about 60 grams (g), about 40 grams (g) to about 45 grams (g), about 40 grams (g) to about 50 grams (g), about 40 grams (g) to about 60 grams (g), about 45 grams (g) to about 50 grams (g), about 45 grams (g) to about 60 grams (g), or about 50 grams (g) to about 60 grams (g).

In some embodiments, about 50 milligrams (mg) to about 1,500 milligrams (mg) of the S53 protease of the present disclosure is combined with about 5 grams (g) to about 60 grams (g) of a food protein.

In some embodiments, about 50 milligrams (mg) to about 1,500 milligrams (mg) of the S53 protease of the present disclosure is combined with about 5 grams (g) to about 60 grams (g) of a food protein. In some embodiments, the provided amount of the S53 protease of the present disclosure comprises about 50 milligrams (mg) to about 100 milligrams (mg), about 50 milligrams (mg) to about 150 milligrams (mg), about 50 milligrams (mg) to about 200 milligrams (mg), about 50 milligrams (mg) to about 300 milligrams (mg), about 50 milligrams (mg) to about 400 milligrams (mg), about 50 milligrams (mg) to about 500 milligrams (mg), about 50 milligrams (mg) to about 750 milligrams (mg), about 50 milligrams (mg) to about 1,000 milligrams (mg), about 50 milligrams (mg) to about 1,500 milligrams (mg), about 100 milligrams (mg) to about 150 milligrams (mg), about 100 milligrams (mg) to about 200 milligrams (mg), about 100 milligrams (mg) to about 300 milligrams (mg), about 100 milligrams (mg) to about 400 milligrams (mg), about 100 milligrams (mg) to about 500 milligrams (mg), about 100 milligrams (mg) to about 750 milligrams (mg), about 100 milligrams (mg) to about 1,000 milligrams (mg), about 100 milligrams (mg) to about 1,500 milligrams (mg), about 150 milligrams (mg) to about 200 milligrams (mg), about 150 milligrams (mg) to about 300 milligrams (mg), about 150 milligrams (mg) to about 400 milligrams (mg), about 150 milligrams (mg) to about 500 milligrams (mg), about 150 milligrams (mg) to about 750 milligrams (mg), about 150 milligrams (mg) to about 1,000 milligrams (mg), about 150 milligrams (mg) to about 1,500 milligrams (mg), about 200 milligrams (mg) to about 300 milligrams (mg), about 200 milligrams (mg) to about 400 milligrams (mg), about 200 milligrams (mg) to about 500 milligrams (mg), about 200 milligrams (mg) to about 750 milligrams (mg), about 200 milligrams (mg) to about 1,000 milligrams (mg), about 200 milligrams (mg) to about 1,500 milligrams (mg), about 300 milligrams (mg) to about 400 milligrams (mg), about 300 milligrams (mg) to about 500 milligrams (mg), about 300 milligrams (mg) to about 750 milligrams (mg), about 300 milligrams (mg) to about 1,000 milligrams (mg), about 300 milligrams (mg) to about 1,500 milligrams (mg), about 400 milligrams (mg) to about 500 milligrams (mg), about 400 milligrams (mg) to about 750 milligrams (mg), about 400 milligrams (mg) to about 1,000 milligrams (mg), about 400 milligrams (mg) to about 1,500 milligrams (mg), about 500 milligrams (mg) to about 750 milligrams (mg), about 500 milligrams (mg) to about 1,000 milligrams (mg), about 500 milligrams (mg) to about 1,500 milligrams (mg), about 750 milligrams (mg) to about 1,000 milligrams (mg), about 750 milligrams (mg) to about 1,500 milligrams (mg), or about 1,000 milligrams (mg) to about 1,500 milligrams (mg) and the amount of food protein comprises about 5 grams (g) to about 10 grams (g), about 5 grams (g) to about 15 grams (g), about 5 grams (g) to about 20 grams (g), about 5 grams (g) to about 25 grams (g), about 5 grams (g) to about 30 grams (g), about 5 grams (g) to about 35 grams (g), about 5 grams (g) to about 40 grams (g), about 5 grams (g) to about 45 grams (g), about 5 grams (g) to about 50 grams (g), about 5 grams (g) to about 60 grams (g), about 10 grams (g) to about 15 grams (g), about 10 grams (g) to about 20 grams (g), about 10 grams (g) to about 25 grams (g), about 10 grams (g) to about 30 grams (g), about 10 grams (g) to about 35 grams (g), about 10 grams (g) to about 40 grams (g), about 10 grams (g) to about 45 grams (g), about 10 grams (g) to about 50 grams (g), about 10 grams (g) to about 60 grams (g), about 15 grams (g) to about 20 grams (g), about 15 grams (g) to about 25 grams (g), about 15 grams (g) to about 30 grams (g), about 15 grams (g) to about 35 grams (g), about 15 grams (g) to about 40 grams (g), about 15 grams (g) to about 45 grams (g), about 15 grams (g) to about 50 grams (g), about 15 grams (g) to about 60 grams (g), about 20 grams (g) to about 25 grams (g), about 20 grams (g) to about 30 grams (g), about 20 grams (g) to about 35 grams (g), about 20 grams (g) to about 40 grams (g), about 20 grams (g) to about 45 grams (g), about 20 grams (g) to about 50 grams (g), about 20 grams (g) to about 60 grams (g), about 25 grams (g) to about 30 grams (g), about 25 grams (g) to about 35 grams (g), about 25 grams (g) to about 40 grams (g), about 25 grams (g) to about 45 grams (g), about 25 grams (g) to about 50 grams (g), about 25 grams (g) to about 60 grams (g), about 30 grams (g) to about 35 grams (g), about 30 grams (g) to about 40 grams (g), about 30 grams (g) to about 45 grams (g), about 30 grams (g) to about 50 grams (g), about 30 grams (g) to about 60 grams (g), about 35 grams (g) to about 40 grams (g), about 35 grams (g) to about 45 grams (g), about 35 grams (g) to about 50 grams (g), about 35 grams (g) to about 60 grams (g), about 40 grams (g) to about 45 grams (g), about 40 grams (g) to about 50 grams (g), about 40 grams (g) to about 60 grams (g), about 45 grams (g) to about 50 grams (g), about 45 grams (g) to about 60 grams (g), or about 50 grams (g) to about 60 grams (g).

In some embodiments, about 50 milligrams (mg), about 100 milligrams (mg), about 150 milligrams (mg), about 200 milligrams (mg), about 300 milligrams (mg), about 400 milligrams (mg), about 500 milligrams (mg), about 750 milligrams (mg), about 1,000 milligrams (mg), or about 1,500 milligrams (mg) of the S53 protease of the present disclosure is combined with about 5 grams (g), about 10 grams (g), about 15 grams (g), about 20 grams (g), about 25 grams (g), about 30 grams (g), about 35 grams (g), about 40 grams (g), about 45 grams (g), about 50 grams (g), or about 60 grams (g) of the food protein.

In some embodiments, at least about 50 milligrams (mg), about 100 milligrams (mg), about 150 milligrams (mg), about 200 milligrams (mg), about 300 milligrams (mg), about 400 milligrams (mg), about 500 milligrams (mg), about 750 milligrams (mg), or about 1,000 milligrams (mg) of the S53 protease of the present disclosure is combined with at least about 5 grams (g), about 10 grams (g), about 15 grams (g), about 20 grams (g), about 25 grams (g), about 30 grams (g), about 35 grams (g), about 40 grams (g), about 45 grams (g), or about 50 grams (g) of the food protein.

In some embodiments, at most about 100 milligrams (mg), about 150 milligrams (mg), about 200 milligrams (mg), about 300 milligrams (mg), about 400 milligrams (mg), about 500 milligrams (mg), about 750 milligrams (mg), about 1,000 milligrams (mg), or about 1,500 milligrams (mg) of the S53 protease of the present disclosure is combined with at most about 10 grams (g), about 15 grams (g), about 20 grams (g), about 25 grams (g), about 30 grams (g), about 35 grams (g), about 40 grams (g), about 45 grams (g), about 50 grams (g), or about 60 grams (g) of the food protein.

A food supplement of the disclosure may further comprise components such as a bulking agent, a carrier, a sweetener, a coating, a preservative, a binding agent, a dessicant, a lubricating agent, a filler, a solubilizing agent, an emulsifier, a stabilizer, a matrix modifier, and the like. Examples of bulking agents suitable for use in the present disclosure include gum acacia, gum arabic, xanthan gum, guar gum, and pectin. Example of carriers include maltodextrin, polypropylene, starch, modified starch, gum, proteins, and amino acids. Examples of sweeteners include glucose, fructose, stevia, acesulfame potassium, and erythritol. Examples of coatings include ethyl cellulose, hydroxypropyl methyl cellulose, and shellac. Examples of preservatives include benzoic acid, benzyl alcohol, and calcium acetate. Examples of binding agents include croscarmellose sodium, povidone, and dextrin. Examples of dessicants include silicon dioxide, and calcium silicate. Examples of lubricating agents include magnesium stearate, stearic acid, and silicon dioxide. Examples of fillers include maltodextrin, dextrin, starch, and calcium salts. Examples of solubilizing agents include cyclodextrin, and lecithin. Examples of emulsifiers include vegetable oils, fatty acids and mono-, and di- and triglycerides, such as medium chain triglycerides or their esters. Suitable stabilizers include agar, pectin and lecithin. Suitable matrix modifiers are those with a buffering capacity between pH 1 and pH 6 and known to be suitable for use in food supplements. Examples include salts of weak organic and inorganic acids, such as flavonoids, flavonols, isoflavones, catechins, gallic acid, monohydrate or dihydrate phosphates, sulfates, ascorbates, amino acids, sodium citrate, citric acid, benzoates, gluconic acid, acetic acid, picolinic acid, nicotinic acid, and phenolic or polyphenolic compounds. One of ordinary skill in the art can readily determine the amount of each ingredient to be added to the food supplement.

One of skill will appreciate that the food supplements of the present disclosure can comprise more than one of the herein-disclosed proteases. For example, a food supplement may comprise one, two, three, four, or more herein-disclosed proteases.

An aspect of the present disclosure provides a food supplement including an S53 family protease having an amino acid sequence that is at least 80% identical to SEQ ID NO: 1 and includes at least one mutation in the amino acid sequence's 513-loop which corresponds to amino acids 513 to 517 of SEQ ID NO: 1.

In embodiments, the at least one mutation provides increased thermostability, increased enzymatic activity, and/or increased substrate specificity, relative to an S53 family protease having an amino acid sequence that lacks the at least one mutation located at position 513, 514, 515, and/or 517 with respect to SEQ ID NO: 1.

In embodiments, the S53 family protease includes at least two mutations in the 513-loop relative to SEQ ID NO: 1. In some cases, the at least two mutations are located at positions 513 and 514; at positions 513 and 515; at positions 513 and 517; at positions 514 and 515; at positions 514 and 517; or at positions 515 and 517.

In embodiments, the S53 family protease includes at least three mutations in the 513-loop relative to SEQ ID NO: 1. In some cases, the at least three mutations are located at positions 513, 515, and 517; at positions 513, 514, and 517; at positions 513, 514, and 515; or at positions 514, 515, and 517.

In embodiments, the S53 family protease includes at least four mutations in the 513-loop relative to SEQ ID NO: 1.

In embodiments, the S53 family protease includes five mutations in the 513-loop relative to SEQ ID NO: 1 which provide increased enzymatic activity and/or increased substrate specificity.

In embodiments, a mutation at position 513 includes an A to C substitution, A to D substitution, A to E substitution, A to F substitution, A to G substitution, A to H substitution, A to I substitution, A to K substitution, A to L substitution, A to M substitution, A to N substitution, A to P substitution, A to Q substitution, A to R substitution, A to S substitution, A to T substitution, A to V substitution, A to W substitution, or A to Y substitution.

In embodiments, a mutation at position 514 includes an N to A substitution, N to C substitution, N to D substitution, N to E substitution, N to F substitution, N to G substitution, N to H substitution, N to I substitution, N to K substitution, N to L substitution, N to M substitution, N to Q substitution, N to R substitution, N to S substitution, N to T substitution, N to V substitution, N to W substitution, or N to Y substitution.

In embodiments, a mutation at position 515 includes an R to A substitution, R to C substitution, R to D substitution, R to E substitution, R to F substitution, R to G substitution, R to I substitution, R to K substitution, R to L substitution, R to M substitution, R to N substitution, R to P substitution, R to Q substitution, R to S substitution, R to T substitution, R to V substitution, R to W substitution, or R to Y substitution.

In embodiments, a mutation at position 517 includes an R to A substitution, R to C substitution, R to D substitution, R to E substitution, R to F substitution, R to G substitution, R to H substitution, R to I substitution, R to K substitution, R to L substitution, R to M substitution, R to N substitution, R to P substitution, R to Q substitution, R to S substitution, R to T substitution, R to V substitution, R to W substitution, or R to Y substitution.

In embodiments, the at least one mutation in the amino acid sequence's 513-loop is as shown in one of Table 1 to Table 9.

In embodiments, the S53 family protease further includes one or more additional mutations at position 13, 15, 18, 23, 35, 38, 40, 51, 52, 55, 64, 70, 73, 91, 96, 101, 121, 123, 143, 145, 147, 155, 174, 175, 181, 188, 198, 218, 228, 229, 236, 240, 243, 253, 261, 265, 268, 273, 280, 283, 286, 290, 292, 296, 297, 300, 303, 308, 312, 314, 319, 323, 324, 325, 326, 327, 328, 329, 330, 332, 341, 353, 359, 360, 371, 377, 385, 387, 390, 391, 392, 398, 404, 411, 417, 421, 422, 432, 433, 434, 441, 446, 448, 452, 459, 469, 474, 486, 499, 500, 501, 509, 513, 514, 515, 516, 517, 537, and/or 550 relative to SEQ ID NO: 1. In some cases, the one or more additional mutations includes substitutions selected from K13A, E15A, A18Q, R23A, T35A, Q38E, 140M, G51A, D52G, E55A, L641, V70E, E73K, 191 L, E96D, T101A, V121I, Q123R, V143L, D145E, R147T, L155M, L174M, R175Q, E181G, S188A, T228A, S236S, S240P, T253S, N261S, I283F, N297D, V303I, H308L, I312V, A325T, P326S, S328A, A387G, E391A, R392Q, S404A, S417A, R421H, G433S, V441 L, T459A, P486A, P499A, E500D, R517Q, 1537V, and/or A550P relative to SEQ ID NO: 1. In some cases, the one or more additional mutations includes substitutions selected from A325K; A325L; P326F; P326L; P326W; S328Q; S328H; E391V; E391L; E391I; E391M; S417W; V441W; T459W; T459R; P486Q; and/or R517G relative to SEQ ID NO: 1.

In embodiments, the present disclosure relates to an S53 family protease, further including one or more substrate selectivity mutations wherein the one or more substrate selectivity mutations increase protease substrate selectivity relative to a protease including an amino acid sequence as shown in SEQ ID NO: 1 and wherein the one or more additional mutations is at position 198, 218, 229, 243, 265, 268, 273, 280, 286, 290, 292, 296, 300, 314, 319, 323, 324, 325, 326, 327, 328, 329, 330, 332, 341, 353, 359, 360, 371, 377, 385, 390, 391, 398, 411, 417, 422, 432, 433, 434, 441, 446, 448, 452, 459, 469, 474, 486, 501, 509, 513, 514, 515, 516, and/or 517 relative to SEQ ID NO: 1.

In embodiments, the one or more substrate selectivity mutations includes substitutions selected from D198E, I218L, S229D, Q243G, G265A, E268M, E268T, E268C, E268H, E268Q, V273I, G280R, Y286K, N290T, N290S, D292F, D292Y, L296T, T300S, S314P, G319Q, S323R, W324K, A325K, A325L, P326F, P326W, P326L, A327Y, A327D, S328H, S328Q, I329L, A330F, M332I, M332L, A341 R, S353E, E359V, E359L, Q360C, A371S, A377G, A385V, A385L, I390W, E391V, E391M, E391 L, E391I, D398S, R411Q, R411M, R411E, R411D, S417W, A422V, A432G, G433G, S434I, S434R, S434T, S434V, V441W, D446S, A448N, E452Y, E452W, E452F, T459R, T459W, A469V, A474V, P486Q, V501C, N509H, A513T, A513D, A513Y, N514G, R515L, R515E, R515I, A516T, A516S, and/or R517G relative to SEQ ID NO: 1. In some cases, the S53 family protease further includes one or more substrate selectivity mutations wherein the one or more substrate selectivity mutations increase protease substrate selectivity when casein is a substrate relative to a protease including an amino acid sequence as shown in SEQ ID NO: 1 and wherein the one or more additional mutations is at position 198, 218, 229, 243, 265, 268, 273, 280, 286, 290, 292, 296, 300, 314, 319, 323, 324, 325, 326, 327, 328, 329, 330, 332, 341, 353, 359, 360, 371, 377, 385, 390, 391, 398, 411, 417, 422, 432, 433, 434, 441, 446, 448, 452, 459, 469, 474, 486, 501, 509, 513, 514, 515, 516, and/or 517 relative to SEQ ID NO: 1. In some cases, the one or more substrate selectivity mutations includes substitutions selected from D198E, I218L, S229D, Q243G, G265A, E268M, E268T, E268C, E268H, E268Q, V273I, G280R, Y286K, N290T, N290S, D292F, D292Y, L296T, T300S, S314P, G319Q, S323R, W324K, A325K, A325L, P326F, P326W, P326L, A327D, A327Y, S328Q, S328H, I329L, A330F, M332L, M332I, A341 R, S353E, E359L, E359V, Q360C, A371S, A377G, A385L, A385V, I390W, E391V, E391L, E391I, E391M, D398S, R411D, R411E, R411M, R411Q, S417W, A422V, A432G, G433G, S434I, S434R, S434T, S434V, V441W, D446S, A448N, A448Y, E452Y, E452W, E452F, T459R, T459W, A469V, A474V, P486Q, V501C, N509H, A513Y, A513D, A513T, N514G, R515I, R515E, R515L, A516S, A516T, and/or R517G relative to SEQ ID NO: 1.

In embodiments, the S53 family protease further includes one or more substrate selectivity mutations wherein the one or more substrate selectivity mutations increase protease substrate selectivity when zein is a substrate relative to a protease including an amino acid sequence as shown in SEQ ID NO: 1 and wherein the one or more additional mutations is at position 198, 218, 229, 243, 265, 268, 273, 280, 286, 290, 292, 296, 300, 314, 319, 323, 324, 325, 326, 327, 328, 329, 330, 332, 341, 353, 359, 360, 371, 377, 385, 390, 391, 398, 411, 417, 422, 432, 433, 434, 441, 446, 448, 452, 459, 469, 474, 486, 501, 509, 513, 514, 515, 516, and/or 517 relative to SEQ ID NO: 1. In some cases, the one or more substrate selectivity mutations includes substitutions selected from D198E, I218L, S229D, Q243G, G265A, E268M, E268T, E268C, E268H, E268Q, V273I, G280R, Y286K, N290T, N290S, D292Y, D292F, L296T, T300S, S314P, G319Q, S323R, W324K, A325K, A325L, P326F, P326L, P326W, A327D, A327Y, S328Q, S328H, I329L, A330F, M332I, M332L, A341 R, S353E, E359L, E359V, Q360C, A371S, A377G, A385L, A385V, I390W, E391V, E391M, E391I, E391L, D398S, R411Q, R411M, R411E, R411D, S417W, A422V, A432G, G433G, S434I, S434R, S434T, S434V, V441W, D446S, A448N, E452F, E452W, E452F, T459R, T459W, A469V, A474V, P486Q, V501 C, N509H, A513T, A513D, A513Y, N514G, R515I, R515E, R515L, A516S, A516T, and/or R517G relative to SEQ ID NO: 1.

In embodiments, the amino acid sequence lacks a mutation at position 198, 218, 229, 243, 265, 268, 273, 280, 286, 290, 292, 296, 300, 314, 319, 323, 324, 325, 326, 327, 328, 329, 330, 332, 341, 353, 359, 360, 371, 377, 385, 390, 391, 398, 411, 417, 422, 432, 433, 434, 441, 446, 448, 452, 459, 469, 474, 486, 501, and/or 509 relative to SEQ ID NO: 1.

In embodiments, the amino acid sequence lacks a mutation at position 266, 270, 352, and/or 466 relative to SEQ ID NO: 1.

In embodiments, the amino acid sequence lacks a mutation at each of positions 266, 270, 352, and 466 relative to SEQ ID NO: 1.

In embodiments, the amino acid sequence lacks a mutation position 516 relative to an amino acid SEQ ID NO: 1.

In embodiments, the amino acid sequence includes a mutation position 516 relative to an amino acid SEQ ID NO: 1. In some cases, the mutation at position 516 includes T.

In embodiments, the amino acid sequence is at least 85% identical to SEQ ID NO: 1, is at least 90% identical to SEQ ID NO: 1, is at least 95% identical to SEQ ID NO: 1, is at least 96% identical to SEQ ID NO: 1, is at least 97% identical to SEQ ID NO: 1, is at least 98% identical to SEQ ID NO: 1, or is at least 99% identical to SEQ ID NO: 1 and maintains or increases thermostability, enzymatic activity, and/or substrate specificity relative to an S53 family protease having the amino acid sequence of SEQ ID NO: 1.

In embodiments, the protein is obtained from or naturally present in Adzuki beans, Almonds, Amaranth, Asparagus, Baby Lima, Barley, Beef, Black beans, Black eyed peas, Broccoli, Buckwheat, Cannellini beans, Cashews, Chia seeds, Chicken, Chickpea, Chlorella, Corn, Cowpea, Cranberry beans, Crowder pea, Egg, e.g., chicken egg, Fava beans, Field Peas, Flounder, Great Northern Beans, Green beans, Hemp protein powder, Kamut, Kidney beans, Lady cream peas, Lentil, Lupine Beans, Masoor Dal (Indian Red lentils), Milk or other milk products, e.g., cheese and yoghurt, Mung beans, Navy pea, Pea, Peanut, Pigeon Peas, Pink beans, Pinto beans, Pistachios, Pork, Quinoa, Royal Canin, Rye berries, Salmon, Soy, Spirulina, Sunflower seeds, Turkey, White beans, and Yellow split peas.

### Methods for Improving the Digestion of Proteins in a Food Using Food Supplements Comprising S53 Family Proteases

S53 family proteases in the form of food supplements, e.g., dietary supplements, nutritional supplements, sports nutrition supplements, digestive aid supplements, and the like, can be ingested along with a food, thereby providing increased digestion of the food proteins and enhancing a food's protein bioavailability.

The food supplements may be in various dosage forms, including for example, tablet, capsule, powder, granule, pellet, soft gel, hard gel, controlled release form, liquid, syrup, suspension, emulsion, and the like. Any commercially acceptable formulation known to be suitable for use in food products may be used in the food supplements of the present disclosure.

As noted above, the present disclosure is based, at least in part, on the discovery of S53 family proteases that have increased thermostability, increased enzymatic activity, and/or increased substrate specificity in digesting target food proteins. The food supplement may be designed to be ingested with the food comprising the target food protein or may be ingested just before or just after the food, typically within two hours before or after ingesting the food. Thus, for the purposes of the present disclosure, a protease of the present disclosure, or a food supplement comprising the protease, is “ingested with” a food, if it is ingested simultaneously with the food or within two hours before or after ingestion of the food.

One of skill will appreciate that the food supplements of the present disclosure can comprise more than one of the herein-disclosed proteases. For example, a food supplement may comprise one, two, three, four, or more herein-disclosed proteases.

An aspect of the present disclosure provides a method for improving the digestion of proteins in a food by a subject, the method including ingesting with the food, a food supplement including an S53 family protease having an amino acid sequence that is at least 80% identical to SEQ ID NO: 1 and includes at least one mutation in the amino acid sequence's 513-loop which corresponds to amino acids 513 to 517 of SEQ ID NO: 1.

In embodiments, the at least one mutation provides increased thermostability, increased enzymatic activity, and/or increased substrate specificity, relative to an S53 family protease having an amino acid sequence that lacks the at least one mutation located at position 513, 514, 515, and/or 517 with respect to SEQ ID NO: 1.

In embodiments, the S53 family protease includes at least two mutations in the 513-loop relative to SEQ ID NO: 1. In some cases, the at least two mutations are located at positions 513 and 514; at positions 513 and 515; at positions 513 and 517; at positions 514 and 515; at positions 514 and 517; or at positions 515 and 517.

In embodiments, the S53 family protease includes at least three mutations in the 513-loop relative to SEQ ID NO: 1. In some cases, the at least three mutations are located at positions 513, 515, and 517; at positions 513, 514, and 517; at positions 513, 514, and 515; or at positions 514, 515, and 517.

In embodiments, the S53 family protease includes at least four mutations in the 513-loop relative to SEQ ID NO: 1.

In embodiments, the S53 family protease includes five mutations in the 513-loop relative to SEQ ID NO: 1 which provide increased enzymatic activity and/or increased substrate specificity.

In embodiments, a mutation at position 513 includes an A to C substitution, A to D substitution, A to E substitution, A to F substitution, A to G substitution, A to H substitution, A to I substitution, A to K substitution, A to L substitution, A to M substitution, A to N substitution, A to P substitution, A to Q substitution, A to R substitution, A to S substitution, A to T substitution, A to V substitution, A to W substitution, or A to Y substitution.

In embodiments, a mutation at position 514 includes an N to A substitution, N to C substitution, N to D substitution, N to E substitution, N to F substitution, N to G substitution, N to H substitution, N to I substitution, N to K substitution, N to L substitution, N to M substitution, N to Q substitution, N to R substitution, N to S substitution, N to T substitution, N to V substitution, N to W substitution, or N to Y substitution.

In embodiments, a mutation at position 515 includes an R to A substitution, R to C substitution, R to D substitution, R to E substitution, R to F substitution, R to G substitution, R to I substitution, R to K substitution, R to L substitution, R to M substitution, R to N substitution, R to P substitution, R to Q substitution, R to S substitution, R to T substitution, R to V substitution, R to W substitution, or R to Y substitution.

In embodiments, a mutation at position 517 includes an R to A substitution, R to C substitution, R to D substitution, R to E substitution, R to F substitution, R to G substitution, R to H substitution, R to I substitution, R to K substitution, R to L substitution, R to M substitution, R to N substitution, R to P substitution, R to Q substitution, R to S substitution, R to T substitution, R to V substitution, R to W substitution, or R to Y substitution.

In embodiments, the at least one mutation in the amino acid sequence's 513-loop is as shown in one of Table 1 to Table 9.

In embodiments, the S53 family protease further includes one or more additional mutations at position 13, 15, 18, 23, 35, 38, 40, 51, 52, 55, 64, 70, 73, 91, 96, 101, 121, 123, 143, 145, 147, 155, 174, 175, 181, 188, 198, 218, 228, 229, 236, 240, 243, 253, 261, 265, 268, 273, 280, 283, 286, 290, 292, 296, 297, 300, 303, 308, 312, 314, 319, 323, 324, 325, 326, 327, 328, 329, 330, 332, 341, 353, 359, 360, 371, 377, 385, 387, 390, 391, 392, 398, 404, 411, 417, 421, 422, 432, 433, 434, 441, 446, 448, 452, 459, 469, 474, 486, 499, 500, 501, 509, 513, 514, 515, 516, 517, 537, and/or 550 relative to SEQ ID NO: 1. In some cases, the one or more additional mutations includes substitutions selected from K13A, E15A, A18Q, R23A, T35A, Q38E, 140M, G51A, D52G, E55A, L641, V70E, E73K, 191 L, E96D, T101A, V121I, Q123R, V143L, D145E, R147T, L155M, L174M, R175Q, E181G, S188A, T228A, S236S, S240P, T253S, N261S, I283F, N297D, V303I, H308L, I312V, A325T, P326S, S328A, A387G, E391A, R392Q, S404A, S417A, R421H, G433S, V441 L, T459A, P486A, P499A, E500D, R517Q, 1537V, and/or A550P relative to SEQ ID NO: 1. In some cases, the one or more additional mutations includes substitutions selected from A325K; A325L; P326F; P326L; P326W; S328Q; S328H; E391V; E391L; E391I; E391M; S417W; V441W; T459W; T459R; P486Q; and/or R517G relative to SEQ ID NO: 1.

In embodiments, the present disclosure relates to an S53 family protease, further including one or more substrate selectivity mutations wherein the one or more substrate selectivity mutations increase protease substrate selectivity relative to a protease including an amino acid sequence as shown in SEQ ID NO: 1 and wherein the one or more additional mutations is at position 198, 218, 229, 243, 265, 268, 273, 280, 286, 290, 292, 296, 300, 314, 319, 323, 324, 325, 326, 327, 328, 329, 330, 332, 341, 353, 359, 360, 371, 377, 385, 390, 391, 398, 411, 417, 422, 432, 433, 434, 441, 446, 448, 452, 459, 469, 474, 486, 501, 509, 513, 514, 515, 516, and/or 517 relative to SEQ ID NO: 1.

In embodiments, the one or more substrate selectivity mutations includes substitutions selected from D198E, I218L, S229D, Q243G, G265A, E268M, E268T, E268C, E268H, E268Q, V273I, G280R, Y286K, N290T, N290S, D292F, D292Y, L296T, T300S, S314P, G319Q, S323R, W324K, A325K, A325L, P326F, P326W, P326L, A327Y, A327D, S328H, S328Q, I329L, A330F, M332I, M332L, A341 R, S353E, E359V, E359L, Q360C, A371S, A377G, A385V, A385L, I390W, E391V, E391M, E391 L, E391I, D398S, R411Q, R411M, R411E, R411D, S417W, A422V, A432G, G433G, S434I, S434R, S434T, S434V, V441W, D446S, A448N, E452Y, E452W, E452F, T459R, T459W, A469V, A474V, P486Q, V501C, N509H, A513T, A513D, A513Y, N514G, R515L, R515E, R515I, A516T, A516S, and/or R517G relative to SEQ ID NO: 1. In some cases, the S53 family protease further includes one or more substrate selectivity mutations wherein the one or more substrate selectivity mutations increase protease substrate selectivity when casein is a substrate relative to a protease including an amino acid sequence as shown in SEQ ID NO: 1 and wherein the one or more additional mutations is at position 198, 218, 229, 243, 265, 268, 273, 280, 286, 290, 292, 296, 300, 314, 319, 323, 324, 325, 326, 327, 328, 329, 330, 332, 341, 353, 359, 360, 371, 377, 385, 390, 391, 398, 411, 417, 422, 432, 433, 434, 441, 446, 448, 452, 459, 469, 474, 486, 501, 509, 513, 514, 515, 516, and/or 517 relative to SEQ ID NO: 1. In some cases, the one or more substrate selectivity mutations includes substitutions selected from D198E, I218L, S229D, Q243G, G265A, E268M, E268T, E268C, E268H, E268Q, V273I, G280R, Y286K, N290T, N290S, D292F, D292Y, L296T, T300S, S314P, G319Q, S323R, W324K, A325K, A325L, P326F, P326W, P326L, A327D, A327Y, S328Q, S328H, I329L, A330F, M332L, M332I, A341 R, S353E, E359L, E359V, Q360C, A371S, A377G, A385L, A385V, I390W, E391V, E391L, E391I, E391M, D398S, R411D, R411E, R411M, R411Q, S417W, A422V, A432G, G433G, S434I, S434R, S434T, S434V, V441W, D446S, A448N, A448Y, E452Y, E452W, E452F, T459R, T459W, A469V, A474V, P486Q, V501C, N509H, A513Y, A513D, A513T, N514G, R515I, R515E, R515L, A516S, A516T, and/or R517G relative to SEQ ID NO: 1.

In embodiments, the S53 family protease further includes one or more substrate selectivity mutations wherein the one or more substrate selectivity mutations increase protease substrate selectivity when zein is a substrate relative to a protease including an amino acid sequence as shown in SEQ ID NO: 1 and wherein the one or more additional mutations is at position 198, 218, 229, 243, 265, 268, 273, 280, 286, 290, 292, 296, 300, 314, 319, 323, 324, 325, 326, 327, 328, 329, 330, 332, 341, 353, 359, 360, 371, 377, 385, 390, 391, 398, 411, 417, 422, 432, 433, 434, 441, 446, 448, 452, 459, 469, 474, 486, 501, 509, 513, 514, 515, 516, and/or 517 relative to SEQ ID NO: 1. In some cases, the one or more substrate selectivity mutations includes substitutions selected from D198E, I218L, S229D, Q243G, G265A, E268M, E268T, E268C, E268H, E268Q, V273I, G280R, Y286K, N290T, N290S, D292Y, D292F, L296T, T300S, S314P, G319Q, S323R, W324K, A325K, A325L, P326F, P326L, P326W, A327D, A327Y, S328Q, S328H, I329L, A330F, M332I, M332L, A341 R, S353E, E359L, E359V, Q360C, A371S, A377G, A385L, A385V, I390W, E391V, E391M, E391I, E391L, D398S, R411Q, R411M, R411E, R411D, S417W, A422V, A432G, G433G, S434I, S434R, S434T, S434V, V441W, D446S, A448N, E452F, E452W, E452F, T459R, T459W, A469V, A474V, P486Q, V501 C, N509H, A513T, A513D, A513Y, N514G, R515I, R515E, R515L, A516S, A516T, and/or R517G relative to SEQ ID NO: 1.

In embodiments, the amino acid sequence lacks a mutation at position 198, 218, 229, 243, 265, 268, 273, 280, 286, 290, 292, 296, 300, 314, 319, 323, 324, 325, 326, 327, 328, 329, 330, 332, 341, 353, 359, 360, 371, 377, 385, 390, 391, 398, 411, 417, 422, 432, 433, 434, 441, 446, 448, 452, 459, 469, 474, 486, 501, and/or 509 relative to SEQ ID NO: 1.

In embodiments, the amino acid sequence lacks a mutation at position 266, 270, 352, and/or 466 relative to SEQ ID NO: 1.

In embodiments, the amino acid sequence lacks a mutation at each of positions 266, 270, 352, and 466 relative to SEQ ID NO: 1.

In embodiments, the amino acid sequence lacks a mutation position 516 relative to an amino acid SEQ ID NO: 1.

In embodiments, the amino acid sequence includes a mutation position 516 relative to an amino acid SEQ ID NO: 1. In some cases, the mutation at position 516 includes T.

In embodiments, the amino acid sequence is at least 85% identical to SEQ ID NO: 1, is at least 90% identical to SEQ ID NO: 1, is at least 95% identical to SEQ ID NO: 1, is at least 96% identical to SEQ ID NO: 1, is at least 97% identical to SEQ ID NO: 1, is at least 98% identical to SEQ ID NO: 1, or is at least 99% identical to SEQ ID NO: 1 and maintains or increases thermostability, enzymatic activity, and/or substrate specificity relative to an S53 family protease having the amino acid sequence of SEQ ID NO: 1.

In embodiments, the protein is obtained from or naturally present in Adzuki beans, Almonds, Amaranth, Asparagus, Baby Lima, Barley, Beef, Black beans, Black eyed peas, Broccoli, Buckwheat, Cannellini beans, Cashews, Chia seeds, Chicken, Chickpea, Chlorella, Corn, Cowpea, Cranberry beans, Crowder pea, Egg, e.g., chicken egg, Fava beans, Field Peas, Flounder, Great Northern Beans, Green beans, Hemp protein powder, Kamut, Kidney beans, Lady cream peas, Lentil, Lupine Beans, Masoor Dal (Indian Red lentils), Milk or other milk products, e.g., cheese and yoghurt, Mung beans, Navy pea, Pea, Peanut, Pigeon Peas, Pink beans, Pinto beans, Pistachios, Pork, Quinoa, Royal Canin, Rye berries, Salmon, Soy, Spirulina, Sunflower seeds, Turkey, White beans, and Yellow split peas.

Methods for degrading proteins using S53 family proteases

An aspect of the present disclosure provides a method for degrading a protein, the method including contacting the protein with an S53 family protease having an amino acid sequence that is at least 80% identical to SEQ ID NO: 1 and includes at least one mutation in the amino acid sequence's 513-loop which corresponds to amino acids 513 to 517 of SEQ ID NO: 1.

In embodiments, the at least one mutation provides increased thermostability, increased enzymatic activity, and/or increased substrate specificity, relative to an S53 family protease having an amino acid sequence that lacks the at least one mutation located at position 513, 514, 515, and/or 517 with respect to SEQ ID NO: 1.

In embodiments, the S53 family protease includes at least two mutations in the 513-loop relative to SEQ ID NO: 1. In some cases, the at least two mutations are located at positions 513 and 514; at positions 513 and 515; at positions 513 and 517; at positions 514 and 515; at positions 514 and 517; or at positions 515 and 517.

In embodiments, the S53 family protease includes at least three mutations in the 513-loop relative to SEQ ID NO: 1. In some cases, the at least three mutations are located at positions 513, 515, and 517; at positions 513, 514, and 517; at positions 513, 514, and 515; or at positions 514, 515, and 517.

In embodiments, the S53 family protease includes at least four mutations in the 513-loop relative to SEQ ID NO: 1.

In embodiments, the S53 family protease includes five mutations in the 513-loop relative to SEQ ID NO: 1 which provide increased enzymatic activity and/or increased substrate specificity.

In embodiments, a mutation at position 513 includes an A to C substitution, A to D substitution, A to E substitution, A to F substitution, A to G substitution, A to H substitution, A to I substitution, A to K substitution, A to L substitution, A to M substitution, A to N substitution, A to P substitution, A to Q substitution, A to R substitution, A to S substitution, A to T substitution, A to V substitution, A to W substitution, or A to Y substitution.

In embodiments, a mutation at position 514 includes an N to A substitution, N to C substitution, N to D substitution, N to E substitution, N to F substitution, N to G substitution, N to H substitution, N to I substitution, N to K substitution, N to L substitution, N to M substitution, N to Q substitution, N to R substitution, N to S substitution, N to T substitution, N to V substitution, N to W substitution, or N to Y substitution.

In embodiments, a mutation at position 515 includes an R to A substitution, R to C substitution, R to D substitution, R to E substitution, R to F substitution, R to G substitution, R to I substitution, R to K substitution, R to L substitution, R to M substitution, R to N substitution, R to P substitution, R to Q substitution, R to S substitution, R to T substitution, R to V substitution, R to W substitution, or R to Y substitution.

In embodiments, a mutation at position 517 includes an R to A substitution, R to C substitution, R to D substitution, R to E substitution, R to F substitution, R to G substitution, R to H substitution, R to I substitution, R to K substitution, R to L substitution, R to M substitution, R to N substitution, R to P substitution, R to Q substitution, R to S substitution, R to T substitution, R to V substitution, R to W substitution, or R to Y substitution.

In embodiments, the at least one mutation in the amino acid sequence's 513-loop is as shown in one of Table 1 to Table 9.

In embodiments, the S53 family protease further includes one or more additional mutations at position 13, 15, 18, 23, 35, 38, 40, 51, 52, 55, 64, 70, 73, 91, 96, 101, 121, 123, 143, 145, 147, 155, 174, 175, 181, 188, 198, 218, 228, 229, 236, 240, 243, 253, 261, 265, 268, 273, 280, 283, 286, 290, 292, 296, 297, 300, 303, 308, 312, 314, 319, 323, 324, 325, 326, 327, 328, 329, 330, 332, 341, 353, 359, 360, 371, 377, 385, 387, 390, 391, 392, 398, 404, 411, 417, 421, 422, 432, 433, 434, 441, 446, 448, 452, 459, 469, 474, 486, 499, 500, 501, 509, 513, 514, 515, 516, 517, 537, and/or 550 relative to SEQ ID NO: 1. In some cases, the one or more additional mutations includes substitutions selected from K13A, E15A, A18Q, R23A, T35A, Q38E, 140M, G51A, D52G, E55A, L641, V70E, E73K, 191 L, E96D, T101A, V121I, Q123R, V143L, D145E, R147T, L155M, L174M, R175Q, E181G, S188A, T228A, S236S, S240P, T253S, N261S, I283F, N297D, V303I, H308L, I312V, A325T, P326S, S328A, A387G, E391A, R392Q, S404A, S417A, R421H, G433S, V441 L, T459A, P486A, P499A, E500D, R517Q, 1537V, and/or A550P relative to SEQ ID NO: 1. In some cases, the one or more additional mutations includes substitutions selected from A325K; A325L; P326F; P326L; P326W; S328Q; S328H; E391V; E391L; E391I; E391M; S417W; V441W; T459W; T459R; P486Q; and/or R517G relative to SEQ ID NO: 1.

In embodiments, the present disclosure relates to an S53 family protease, further including one or more substrate selectivity mutations wherein the one or more substrate selectivity mutations increase protease substrate selectivity relative to a protease including an amino acid sequence as shown in SEQ ID NO: 1 and wherein the one or more additional mutations is at position 198, 218, 229, 243, 265, 268, 273, 280, 286, 290, 292, 296, 300, 314, 319, 323, 324, 325, 326, 327, 328, 329, 330, 332, 341, 353, 359, 360, 371, 377, 385, 390, 391, 398, 411, 417, 422, 432, 433, 434, 441, 446, 448, 452, 459, 469, 474, 486, 501, 509, 513, 514, 515, 516, and/or 517 relative to SEQ ID NO: 1.

In embodiments, the one or more substrate selectivity mutations includes substitutions selected from D198E, I218L, S229D, Q243G, G265A, E268M, E268T, E268C, E268H, E268Q, V273I, G280R, Y286K, N290T, N290S, D292F, D292Y, L296T, T300S, S314P, G319Q, S323R, W324K, A325K, A325L, P326F, P326W, P326L, A327Y, A327D, S328H, S328Q, I329L, A330F, M332I, M332L, A341 R, S353E, E359V, E359L, Q360C, A371S, A377G, A385V, A385L, I390W, E391V, E391M, E391 L, E391I, D398S, R411Q, R411M, R411E, R411D, S417W, A422V, A432G, G433G, S434I, S434R, S434T, S434V, V441W, D446S, A448N, E452Y, E452W, E452F, T459R, T459W, A469V, A474V, P486Q, V501C, N509H, A513T, A513D, A513Y, N514G, R515L, R515E, R515I, A516T, A516S, and/or R517G relative to SEQ ID NO: 1. In some cases, the S53 family protease further includes one or more substrate selectivity mutations wherein the one or more substrate selectivity mutations increase protease substrate selectivity when casein is a substrate relative to a protease including an amino acid sequence as shown in SEQ ID NO: 1 and wherein the one or more additional mutations is at position 198, 218, 229, 243, 265, 268, 273, 280, 286, 290, 292, 296, 300, 314, 319, 323, 324, 325, 326, 327, 328, 329, 330, 332, 341, 353, 359, 360, 371, 377, 385, 390, 391, 398, 411, 417, 422, 432, 433, 434, 441, 446, 448, 452, 459, 469, 474, 486, 501, 509, 513, 514, 515, 516, and/or 517 relative to SEQ ID NO: 1. In some cases, the one or more substrate selectivity mutations includes substitutions selected from D198E, I218L, S229D, Q243G, G265A, E268M, E268T, E268C, E268H, E268Q, V273I, G280R, Y286K, N290T, N290S, D292F, D292Y, L296T, T300S, S314P, G319Q, S323R, W324K, A325K, A325L, P326F, P326W, P326L, A327D, A327Y, S328Q, S328H, I329L, A330F, M332L, M332I, A341 R, S353E, E359L, E359V, Q360C, A371S, A377G, A385L, A385V, I390W, E391V, E391L, E391I, E391M, D398S, R411D, R411E, R411M, R411Q, S417W, A422V, A432G, G433G, S434I, S434R, S434T, S434V, V441W, D446S, A448N, A448Y, E452Y, E452W, E452F, T459R, T459W, A469V, A474V, P486Q, V501C, N509H, A513Y, A513D, A513T, N514G, R515I, R515E, R515L, A516S, A516T, and/or R517G relative to SEQ ID NO: 1.

In embodiments, the S53 family protease further includes one or more substrate selectivity mutations wherein the one or more substrate selectivity mutations increase protease substrate selectivity when zein is a substrate relative to a protease including an amino acid sequence as shown in SEQ ID NO: 1 and wherein the one or more additional mutations is at position 198, 218, 229, 243, 265, 268, 273, 280, 286, 290, 292, 296, 300, 314, 319, 323, 324, 325, 326, 327, 328, 329, 330, 332, 341, 353, 359, 360, 371, 377, 385, 390, 391, 398, 411, 417, 422, 432, 433, 434, 441, 446, 448, 452, 459, 469, 474, 486, 501, 509, 513, 514, 515, 516, and/or 517 relative to SEQ ID NO: 1. In some cases, the one or more substrate selectivity mutations includes substitutions selected from D198E, I218L, S229D, Q243G, G265A, E268M, E268T, E268C, E268H, E268Q, V273I, G280R, Y286K, N290T, N290S, D292Y, D292F, L296T, T300S, S314P, G319Q, S323R, W324K, A325K, A325L, P326F, P326L, P326W, A327D, A327Y, S328Q, S328H, I329L, A330F, M332I, M332L, A341 R, S353E, E359L, E359V, Q360C, A371S, A377G, A385L, A385V, I390W, E391V, E391M, E391I, E391L, D398S, R411Q, R411M, R411E, R411D, S417W, A422V, A432G, G433G, S434I, S434R, S434T, S434V, V441W, D446S, A448N, E452F, E452W, E452F, T459R, T459W, A469V, A474V, P486Q, V501 C, N509H, A513T, A513D, A513Y, N514G, R515I, R515E, R515L, A516S, A516T, and/or R517G relative to SEQ ID NO: 1.

In embodiments, the amino acid sequence lacks a mutation at position 198, 218, 229, 243, 265, 268, 273, 280, 286, 290, 292, 296, 300, 314, 319, 323, 324, 325, 326, 327, 328, 329, 330, 332, 341, 353, 359, 360, 371, 377, 385, 390, 391, 398, 411, 417, 422, 432, 433, 434, 441, 446, 448, 452, 459, 469, 474, 486, 501, and/or 509 relative to SEQ ID NO: 1.

In embodiments, the amino acid sequence lacks a mutation at position 266, 270, 352, and/or 466 relative to SEQ ID NO: 1.

In embodiments, the amino acid sequence lacks a mutation at each of positions 266, 270, 352, and 466 relative to SEQ ID NO: 1.

In embodiments, the amino acid sequence lacks a mutation at position 516 relative to an amino acid SEQ ID NO: 1.

In embodiments, the amino acid sequence includes a mutation at position 516 relative to an amino acid SEQ ID NO: 1. In some cases, the mutation at position 516 includes T.

In embodiments, the amino acid sequence is at least 85% identical to SEQ ID NO: 1, is at least 90% identical to SEQ ID NO: 1, is at least 95% identical to SEQ ID NO: 1, is at least 96% identical to SEQ ID NO: 1, is at least 97% identical to SEQ ID NO: 1, is at least 98% identical to SEQ ID NO: 1, or is at least 99% identical to SEQ ID NO: 1 and maintains or increases thermostability, enzymatic activity, and/or substrate specificity relative to an S53 family protease having the amino acid sequence of SEQ ID NO: 1.

In embodiments, the protein is obtained from or naturally present in Adzuki beans, Almonds, Amaranth, Asparagus, Baby Lima, Barley, Beef, Black beans, Black eyed peas, Broccoli, Buckwheat, Cannellini beans, Cashews, Chia seeds, Chicken, Chickpea, Chlorella, Corn, Cowpea, Cranberry beans, Crowder pea, Egg, e.g., chicken egg, Fava beans, Field Peas, Flounder, Great Northern Beans, Green beans, Hemp protein powder, Kamut, Kidney beans, Lady cream peas, Lentil, Lupine Beans, Masoor Dal (Indian Red lentils), Milk or other milk products, e.g., cheese and yoghurt, Mung beans, Navy pea, Pea, Peanut, Pigeon Peas, Pink beans, Pinto beans, Pistachios, Pork, Quinoa, Royal Canin, Rye berries, Salmon, Soy, Spirulina, Sunflower seeds, Turkey, White beans, and Yellow split peas.

Any protease, food product food supplement, or method disclosed herein is applicable to any herein-disclosed protease, food product food supplement, or method. In other words, any aspect or embodiment described herein can be combined with any other aspect or embodiment as disclosed herein.

### Definitions

Prior to setting forth this disclosure in more detail, it may be helpful to an understanding thereof to provide definitions of certain terms to be used herein.

Unless defined otherwise, all technical and scientific terms used herein have the same meaning as commonly understood by those of ordinary skill in the art to which the disclosure belongs. Although any methods and materials similar or equivalent to those described herein can be used in the practice or testing of particular embodiments, preferred embodiments of compositions, methods and materials are described herein. For the purposes of the present disclosure, the following terms are defined below. Additional definitions are as set forth throughout this disclosure.

As used herein, the singular forms “a”, “an”, and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps. As also used herein, in any instance or embodiment described herein, “comprising” may be replaced with “consisting essentially of” and/or “consisting of” used herein, in any instance or embodiment described.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about,” when immediately preceding a number or numeral, means that the number or numeral ranges plus or minus 10%.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

As used herein, the term “isolated” means material that is substantially or essentially free from components that normally accompany it in its native state. In particular embodiments, the term “obtained” or “derived” is used synonymously with isolated.

An “increased” or “enhanced”, e.g., as in increased thermostability, increased enzymatic activity, and/or increased substrate specificity, refers to amount of thermostability, enzymatic activity, and/or substrate specificity that is provided by a protease of the present disclosure and includes an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the level of amounts derived from a wild type protease. In some cases, “increased” or “enhanced” means a “statistically significant” amount.

In general, “sequence identity” or “sequence homology” refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (generally nucleotides or amino acids), divided by the total number of symbols in the shorter of the two sequences. The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 80% to 100% and integer values therebetween. Typically, the percent identities between a disclosed sequence and a claimed sequence are at least 80%, at least 85%, at least 90%, at least 95%, or at least 98%.

### EXAMPLES

The following Examples are intended for illustration only and do not limit the scope of the invention

### Example 1: Creation and Characterization of Improved S53 Proteases

S53 family proteases having an amino acid sequence that is at least 80% identical to SEQ ID NO: 1 and includes at least one mutation in the amino acid sequence's 513-loop which corresponds to amino acids 513 to 517 of SEQ ID NO: 1 and, wherein the at least one mutation is located at position 513, 514, 515, and/or 517 were created using standard molecular biology and genetic engineering techniques. For example, site specific modifications are introduced into in the DNA sequence encoding a S53 family protease and the modified DNA is expressed by a suitable host cell.

The modified S53 family protease expressed by the host cell is isolated and its thermostability, enzymatic activity, and/or substrate specificity is characterized. For these, the protease is contacted with a suitable substrate (i.e., target protein) under conditions suitable to assay the modified protease's properties. Control assays with unmodified proteases (e.g., having an amino acid sequence of SEQ ID NO: 1) are run in parallel. In some cases, activity is assayed by a reduction in optical density of a solution comprising the protease and substrate, with the reduction in optical density due to digestion of the substrate protein into more soluble and optically-clear protein fragments. In some cases, substrate specificity is assayed by measuring the quantity of peptides released from a series of test protein substrates relative to the unmodified enzyme. If the mutated enzyme performs better on one substrate but the same or worse on a second substrate relative to the unmodified enzyme, then that shows that the substrate specificity has been changed. In some cases, thermostability is assayed by exposing the proteases to a heat treatment (e.g., 70° to 72° C. for about three minutes) and then assaying the proteases' ability to retain enzymatic activity. When a comparison in thermostability, enzymatic activity, and/or substrate specificity is made between the unmodified protease and the modified protease demonstrates that the modified protease has an at least 5% increase in a property, the protease is identified as being “improved”, as having “increased enzymatic activity, and/or increased substrate specificity, relative to an S53 family protease having an amino acid sequence that lacks the at least one mutation located at position 513, 514, 515, and/or 517 with respect to SEQ ID NO: 1”, and the like.

Experimental evidence is displayed in the below Table 1 to Table 9.

TABLE 1
A513 loop mutants having an at least 5% increased activity relative to
an enzyme of SEQ ID NO: 1 (comprising ANRAR) when substrate is casein:

| | | | | | Average | | | | |
|---|---|---|---|---|---|---|---|---|---|
| 513 | 514 | 515 | 516 | 517 | Activity | charges | weights | logPs | hydrophobicity |
| W | R | W | A | Y | 0.104289 | 0.995105 | 780.887 | 1.01697 | −0.03 |
| W | R | W | A | Y | 0.117178 | 0.995105 | 780.887 | 1.01697 | −0.03 |
| Y | N | R | T | R | 0.145433 | 1.991455 | 708.778 | −4.93146 | −5.63 |
| W | F | C | A | G | 0.146078 | −0.04872 | 582.683 | −0.1147 | 3.39 |
| Y | W | R | A | G | 0.149189 | 0.991465 | 651.725 | −1.07563 | −0.36 |
| Y | T | C | A | R | 0.150011 | 0.946775 | 612.71 | −3.12043 | −1.41 |
| N | E | W | A | A | 0.151389 | −1.01245 | 589.606 | −2.1585 | 0.53 |
| Y | N | R | T | R | 0.153622 | 1.991455 | 708.778 | −4.93146 | −5.63 |
| W | A | K | A | R | 0.160089 | 1.995792 | 630.751 | −1.50643 | −1.98 |
| Y | N | R | T | R | 0.162256 | 1.991455 | 708.778 | −4.93146 | −5.63 |
| W | G | G | A | W | 0.163456 | −0.00403 | 575.626 | 0.0682 | 3.2 |
| Y | N | R | T | R | 0.163967 | 1.991455 | 708.778 | −4.93146 | −5.63 |
| Y | N | R | T | R | 0.164478 | 1.991455 | 708.778 | −4.93146 | −5.63 |
| F | G | W | A | T | 0.164544 | −0.00732 | 580.642 | −0.6637 | 3.05 |
| W | S | A | A | R | 0.164767 | 0.995954 | 589.654 | −2.64313 | −0.66 |
| F | G | L | A | R | 0.165133 | 0.992653 | 562.672 | −1.45913 | 0.82 |
| T | W | R | A | T | 0.166222 | 0.992159 | 633.707 | −2.89373 | −1.2 |
| Y | N | R | T | R | 0.167311 | 1.991455 | 708.778 | −4.93146 | −5.63 |
| A | R | W | A | M | 0.167844 | 0.997978 | 633.776 | −0.88233 | 0.16 |
| A | N | R | A | R | 0.176333 | 1.997971 | 586.655 | −5.22076 | −4.6 |

TABLE 2
A513 loop mutants having an at least 5% increase
activity relative to an enzyme of SEQ ID NO: 1
(comprising ANRAR) when substrate is casein:

| | | | | | | ACTIVITY |
|---|---|---|---|---|---|---|
| | | | | | CASEIN | INCREASE |
| 513 | 514 | 515 | 516 | 517 | AVE | (%) |
| W | R | W | A | Y | 0.111 | 43 |
| W | F | C | A | G | 0.146 | 24 |
| Y | W | R | A | G | 0.149 | 23 |
| Y | T | C | A | R | 0.150 | 22 |
| N | E | W | A | A | 0.151 | 21 |
| W | A | K | A | R | 0.160 | 17 |
| W | G | G | A | W | 0.163 | 15 |
| F | G | W | A | T | 0.165 | 15 |
| W | S | A | A | R | 0.165 | 14 |
| F | G | L | A | R | 0.165 | 14 |
| T | W | R | A | T | 0.166 | 14 |
| A | R | W | A | M | 0.168 | 13 |
| G | W | M | A | G | 0.171 | 11 |
| F | E | F | A | D | 0.172 | 11 |
| Y | N | R | T | R | 0.173 | 10 |
| W | A | I | A | K | 0.174 | 10 |
| W | R | Y | A | P | 0.174 | 9 |
| A | D | Y | A | G | 0.175 | 9 |
| V | C | W | A | N | 0.176 | 9 |
| Y | A | Y | A | Y | 0.176 | 8 |
| F | Q | Y | A | G | 0.177 | 8 |
| R | A | W | A | L | 0.177 | 8 |
| H | C | W | A | F | 0.180 | 7 |
| S | D | R | A | W | 0.180 | 6 |
| D | W | I | A | D | 0.180 | 6 |
| F | S | C | A | R | 0.180 | 6 |
| E | R | Y | A | H | 0.182 | 6 |
| E | T | A | A | R | 0.183 | 5 |
| D | R | L | A | G | 0.183 | 5 |
| A | N | R | A | R | 0.193 | 0 |

TABLE 3
A513 loop mutants having an at least 5% increase activity relative to
an enzyme of SEQ ID NO: 1 (comprising ANRAR) when substrate is zein:

| | | | | | Average | | | | |
|---|---|---|---|---|---|---|---|---|---|
| 513 | 514 | 515 | 516 | 517 | Activity | charges | weights | logPs | hydrophobicity |
| W | R | W | A | Y | 0.1535 | 0.995105 | 780.887 | 1.01697 | −0.03 |
| G | W | M | A | G | 0.1622 | −0.00248 | 520.612 | −0.9027 | 3.03 |
| W | R | W | A | Y | 0.167267 | 0.995105 | 780.887 | 1.01697 | −0.03 |
| N | E | W | A | A | 0.1702 | −1.01245 | 589.606 | −2.1585 | 0.53 |
| Q | M | Y | A | G | 0.173167 | −0.00819 | 568.653 | −2.0443 | 1.15 |
| A | D | Y | A | G | 0.177733 | −1.00214 | 495.489 | −2.5683 | 1.08 |
| Y | A | Y | A | Y | 0.178967 | −0.01024 | 649.701 | 0.2222 | 2.02 |
| D | W | I | A | D | 0.1794 | −2.00005 | 618.644 | −0.9231 | 1.01 |
| W | F | C | A | G | 0.180033 | −0.04872 | 582.683 | −0.1147 | 3.39 |
| S | T | D | A | G | 0.181 | −1.00628 | 449.417 | −5.1634 | −0.03 |
| N | D | L | A | G | 0.1812 | −1.0135 | 488.498 | −3.615 | 0.48 |
| I | L | L | A | G | 0.181333 | −0.00206 | 485.626 | 0.1271 | 4.6 |
| F | Q | Y | A | G | 0.182 | −0.00819 | 584.63 | −1.5547 | 1.7 |
| T | C | F | A | E | 0.182933 | −1.05077 | 569.637 | −2.2245 | 1.31 |
| T | L | V | A | G | 0.1838 | −0.00786 | 459.544 | −1.9283 | 3.19 |
| Y | Q | V | A | L | 0.185667 | −0.00853 | 592.694 | −0.7267 | 2.17 |
| D | R | L | A | G | 0.190333 | −0.00077 | 530.583 | −3.22713 | −1.27 |
| M | L | L | A | N | 0.191467 | −0.00612 | 560.718 | −0.9219 | 2.6 |
| W | G | G | A | W | 0.1977 | −0.00403 | 575.626 | 0.0682 | 3.2 |
| F | G | W | A | T | 0.197833 | −0.00732 | 580.642 | −0.6637 | 3.05 |
| F | E | F | A | D | 0.199367 | −2.00485 | 627.651 | −0.8177 | 1.36 |
| S | S | L | A | G | 0.202867 | −0.00701 | 433.462 | −3.9805 | 1.8 |
| F | S | C | A | R | 0.203133 | 0.947969 | 582.684 | −3.21453 | −0.61 |
| D | R | L | A | G | 0.203767 | −0.00077 | 530.583 | −3.22713 | −1.27 |
| Y | W | R | A | G | 0.2041 | 0.991465 | 651.725 | −1.07563 | −0.36 |
| Y | T | C | A | R | 0.208067 | 0.946775 | 612.71 | 3.12043 | −1.41 |
| Y | A | L | A | Y | 0.209833 | −0.00939 | 599.685 | 0.32 | 2.82 |
| A | L | V | A | G | 0.2107 | −0.00202 | 429.518 | −1.2892 | 3.86 |
| R | A | W | A | L | 0.210967 | 0.990982 | 615.736 | −0.58933 | 0.58 |
| L | D | N | A | S | 0.211433 | −1.00177 | 518.524 | −4.2541 | −0.18 |
| E | Q | Q | A | G | 0.211833 | −1.00034 | 531.523 | −4.6154 | −1.34 |
| D | G | I | A | F | 0.212333 | −1.00078 | 521.571 | −1.2477 | 2.77 |
| V | C | G | A | G | 0.213433 | −0.04705 | 405.477 | −2.794 | 2.95 |
| E | F | A | A | Y | 0.2142 | −1.00119 | 599.641 | −0.5669 | 1.95 |
| W | S | T | A | N | 0.214533 | −0.00405 | 577.595 | −3.6701 | 0.42 |
| L | T | G | A | C | 0.214533 | −0.04718 | 463.557 | −2.6545 | 2.4 |
| V | C | W | A | N | 0.215267 | −0.04707 | 591.691 | −1.4574 | 2.02 |
| F | R | Q | A | M | 0.2158 | 0.992657 | 651.791 | −2.11803 | −0.93 |
| Q | F | E | A | Y | 0.215933 | −1.00642 | 656.693 | −1.3213 | 0.48 |
| T | Y | Y | A | N | 0.217867 | −0.00957 | 630.655 | −2.4898 | 0.31 |
| T | L | I | A | Q | 0.2189 | −0.00787 | 544.65 | −1.9041 | 2.16 |
| T | G | G | A | G | 0.219633 | −0.00786 | 361.355 | −4.3676 | 2.01 |
| M | L | I | A | L | 0.2208 | −0.00611 | 559.774 | 1.2488 | 4.76 |
| D | G | C | A | G | 0.220833 | −1.04545 | 421.432 | −3.9753 | 0.97 |
| S | G | T | A | N | 0.2212 | −0.00702 | 448.433 | −5.7627 | 0.09 |
| M | Q | V | A | A | 0.221833 | −0.00611 | 518.637 | −1.9481 | 2.11 |
| F | Q | V | A | A | 0.222367 | −0.00734 | 534.614 | −1.4585 | 2.66 |
| W | A | I | A | K | 0.222767 | 0.995796 | 587.722 | 0.2764 | 1.93 |
| D | G | I | A | N | 0.223133 | −1.00078 | 488.498 | −3.615 | 0.8 |
| M | L | L | A | M | 0.22495 | −0.00611 | 577.814 | 0.9558 | 4.02 |
| C | L | I | A | G | 0.225 | −0.04483 | 475.612 | −0.9892 | 3.83 |
| M | C | M | A | K | 0.225067 | 0.949041 | 582.815 | −1.0776 | 0.69 |
| F | H | F | A | A | 0.225433 | 0.091476 | 591.669 | −0.1715 | 3.22 |
| V | F | R | A | M | 0.226367 | 0.997623 | 622.793 | −0.72753 | 1 |
| G | M | G | A | Y | 0.2267 | −0.00334 | 497.574 | −1.6784 | 2.48 |
| A | F | L | A | E | 0.226967 | −1.00025 | 549.625 | −0.4691 | 2.75 |
| N | E | C | A | Y | 0.228033 | −1.05799 | 598.635 | −3.0243 | −0.35 |
| W | M | P | A | G | 0.230633 | −0.00404 | 560.677 | −0.0279 | 2.67 |
| T | W | R | A | T | 0.230933 | 0.992159 | 633.707 | −2.89373 | −1.2 |
| C | C | V | A | C | 0.231167 | −0.13421 | 497.665 | −2.1972 | 2.57 |
| H | C | W | A | F | 0.231967 | 0.047421 | 662.773 | 0.2197 | 2.51 |
| S | D | R | A | W | 0.232933 | −0.00629 | 633.663 | −3.18833 | −2.18 |
| E | L | A | A | L | 0.2336 | −1.00034 | 515.608 | −0.6657 | 2.62 |
| L | R | Y | A | A | 0.2345 | 0.996663 | 592.698 | −1.36503 | 0.03 |
| Y | A | G | A | Y | 0.235533 | −0.00939 | 543.577 | −1.0947 | 2.24 |
| A | Y | R | A | G | 0.2382 | 0.997131 | 536.59 | −2.77973 | −0.55 |
| F | H | I | A | N | 0.238667 | 0.091465 | 600.677 | −1.5126 | 2.01 |
| C | T | Q | A | G | 0.2393 | −0.04483 | 478.528 | −4.4351 | 0.49 |
| T | M | V | A | E | 0.239767 | −1.00609 | 549.647 | −1.9879 | 1.55 |
| A | R | W | A | M | 0.240267 | 0.997978 | 633.776 | −0.88233 | 0.16 |
| A | L | I | A | N | 0.244433 | −0.00203 | 500.597 | −1.6551 | 2.9 |
| W | S | A | A | R | 0.245267 | 0.995954 | 589.654 | −2.64313 | −0.66 |
| W | R | Y | A | P | 0.245533 | 0.995099 | 691.79 | −0.20083 | −0.72 |
| M | S | Y | A | T | 0.2463 | −0.00694 | 571.653 | −2.5681 | 1.29 |
| H | N | F | A | G | 0.247867 | 0.09212 | 544.569 | −2.9273 | 1.11 |
| L | R | I | A | Y | 0.248033 | 0.996657 | 634.779 | −0.33883 | 0.79 |
| W | R | L | A | N | 0.248533 | 0.99595 | 658.761 | −1.73383 | −0.82 |
| L | F | L | A | S | 0.249 | −0.00249 | 549.669 | −0.3154 | 3.75 |
| S | M | V | A | L | 0.249067 | −0.00701 | 519.665 | −1.1951 | 3.22 |
| N | S | P | A | G | 0.250167 | −0.01423 | 444.445 | −4.6373 | 0.26 |
| V | N | G | A | G | 0.250533 | −0.00237 | 416.435 | −3.8484 | 1.88 |
| A | S | C | A | L | 0.2525 | −0.0467 | 463.557 | −2.6545 | 2.41 |
| Y | N | R | T | R | 0.253467 | 1.991455 | 708.778 | −4.93146 | −5.63 |
| Y | N | R | T | R | 0.2535 | 1.991455 | 708.778 | 4.93146 | −5.63 |
| H | Y | N | A | F | 0.262633 | 0.091254 | 650.693 | −1.6104 | 0.89 |
| Q | G | V | A | K | 0.266567 | 0.992494 | 501.585 | −2.9607 | −0.17 |
| N | V | R | A | C | 0.267867 | 0.94107 | 561.666 | −3.91813 | −1.32 |
| V | R | Y | A | L | 0.2691 | 0.996776 | 620.752 | −0.72893 | 0.49 |
| M | D | N | A | I | 0.269967 | −1.00538 | 562.646 | −2.4933 | 0.96 |
| E | T | A | A | R | 0.270333 | −0.00035 | 546.582 | −4.11383 | −2.08 |
| R | S | S | A | G | 0.272367 | 0.990981 | 476.491 | −5.76333 | −1.79 |
| L | C | I | A | K | 0.2748 | 0.952664 | 546.735 | −0.4916 | 1.85 |
| S | Y | R | A | D | 0.275067 | −0.00715 | 610.625 | −3.96403 | −2.73 |
| A | G | G | A | S | 0.2763 | −0.00202 | 361.355 | −4.3676 | 2.02 |
| V | Y | M | A | R | 0.2782 | 0.996768 | 638.792 | −1.02193 | 0.07 |
| E | N | F | A | H | 0.281233 | −0.90154 | 616.632 | −2.6939 | −0.11 |
| Y | N | R | T | R | 0.2835 | 1.991455 | 708.778 | −4.93146 | −5.63 |
| A | L | G | A | L | 0.283933 | −0.00201 | 443.545 | −0.8991 | 3.84 |
| Y | N | R | T | R | 0.284033 | 1.991455 | 708.778 | −4.93146 | −5.63 |
| L | L | E | A | C | 0.284433 | −1.04541 | 547.675 | −0.7558 | 2.29 |
| N | F | S | A | L | 0.284867 | −0.01423 | 550.613 | −2.4861 | 1.91 |
| T | K | T | A | W | 0.284967 | 0.991981 | 605.693 | −2.028 | −0.17 |
| Y | N | R | T | R | 0.286233 | 1.991455 | 708.778 | −4.93146 | −5.63 |
| Y | N | R | T | R | 0.289167 | 1.991455 | 708.778 | −4.93146 | −5.63 |
| H | S | D | A | C | 0.2893 | −0.95186 | 531.548 | −4.28 | −0.57 |
| Y | N | R | T | R | 0.289733 | 1.991455 | 708.778 | −4.93146 | −5.63 |
| S | G | R | A | C | 0.290467 | 0.948289 | 492.559 | −4.82583 | −1.32 |
| E | R | Y | A | H | 0.291567 | 0.097608 | 674.716 | −2.60043 | −2.79 |
| Y | N | R | T | R | 0.292267 | 1.991455 | 708.778 | −4.93146 | −5.63 |
| R | G | A | A | P | 0.292933 | 0.990969 | 470.531 | −3.22183 | −0.69 |
| R | Y | M | A | V | 0.294233 | 0.990129 | 638.792 | −1.02193 | 0.07 |
| H | D | T | A | N | 0.294833 | −0.90717 | 556.533 | −4.9459 | −1.51 |
| T | V | K | A | G | 0.2962 | 0.991979 | 474.559 | −2.8454 | 0.63 |
| Y | N | R | T | R | 0.296833 | 1.991455 | 708.778 | −4.93146 | −5.63 |
| A | N | R | A | R | 0.312733333 | 1.997970591 | 586.655 | −5.22076 | −4.6 |

TABLE 4
A513 loop mutants having an at least 5% increase activity relative
to SEQ ID NO: 1 (comprising ANRAR) when substrate is zein:

| | | | | | | Activity |
|---|---|---|---|---|---|---|
| | | | | | ZEIN | Increase |
| 513 | 514 | 515 | 516 | 517 | Activity | (%) |
| W | R | W | A | Y | 0.154 | 60 |
| G | W | M | A | G | 0.162 | 57 |
| W | R | W | A | Y | 0.167 | 56 |
| N | E | W | A | A | 0.170 | 55 |
| Q | M | Y | A | G | 0.173 | 55 |
| A | D | Y | A | G | 0.178 | 53 |
| Y | A | Y | A | Y | 0.179 | 53 |
| D | W | I | A | D | 0.179 | 53 |
| W | F | C | A | G | 0.180 | 53 |
| S | T | D | A | G | 0.181 | 52 |
| N | D | L | A | G | 0.181 | 52 |
| I | L | L | A | G | 0.181 | 52 |
| F | Q | Y | A | G | 0.182 | 52 |
| T | C | F | A | E | 0.183 | 52 |
| T | L | V | A | G | 0.184 | 52 |
| Y | Q | V | A | L | 0.186 | 51 |
| D | R | L | A | G | 0.190 | 50 |
| M | L | L | A | N | 0.191 | 50 |
| W | G | G | A | W | 0.198 | 48 |
| F | G | W | A | T | 0.198 | 48 |
| F | E | F | A | D | 0.199 | 48 |
| S | S | L | A | G | 0.203 | 47 |
| F | S | C | A | R | 0.203 | 47 |
| D | R | L | A | G | 0.204 | 47 |
| Y | W | R | A | G | 0.204 | 46 |
| Y | T | C | A | R | 0.208 | 45 |
| Y | A | L | A | Y | 0.210 | 45 |
| A | L | V | A | G | 0.211 | 45 |
| R | A | W | A | L | 0.211 | 45 |
| L | D | N | A | S | 0.211 | 45 |
| E | Q | Q | A | G | 0.212 | 44 |
| D | G | 1 | A | F | 0.212 | 44 |
| V | C | G | A | G | 0.213 | 44 |
| E | F | A | A | Y | 0.214 | 44 |
| L | T | G | A | C | 0.215 | 44 |
| W | S | T | A | N | 0.215 | 44 |
| V | C | W | A | N | 0.215 | 43 |
| F | R | Q | A | M | 0.216 | 43 |
| Q | F | E | A | Y | 0.216 | 43 |
| T | Y | Y | A | N | 0.218 | 43 |
| T | L | I | A | Q | 0.219 | 43 |
| T | G | G | A | G | 0.220 | 42 |
| M | L | I | A | L | 0.221 | 42 |
| D | G | C | A | G | 0.221 | 42 |
| S | G | T | A | N | 0.221 | 42 |
| M | Q | V | A | A | 0.222 | 42 |
| F | Q | V | A | A | 0.222 | 42 |
| W | A | I | A | K | 0.223 | 42 |
| D | G | I | A | N | 0.223 | 41 |
| M | L | L | A | M | 0.225 | 41 |
| C | L | I | A | G | 0.225 | 41 |
| M | C | M | A | K | 0.225 | 41 |
| F | H | F | A | A | 0.225 | 41 |
| V | F | R | A | M | 0.226 | 41 |
| G | M | G | A | Y | 0.227 | 40 |
| A | F | L | A | E | 0.227 | 40 |
| N | E | C | A | Y | 0.228 | 40 |
| W | M | P | A | G | 0.231 | 39 |
| T | W | R | A | T | 0.231 | 39 |
| C | C | V | A | C | 0.231 | 39 |
| H | C | W | A | F | 0.232 | 39 |
| S | D | R | A | W | 0.233 | 39 |
| E | L | A | A | L | 0.234 | 39 |
| L | R | Y | A | A | 0.235 | 38 |
| Y | A | G | A | Y | 0.236 | 38 |
| A | Y | R | A | G | 0.238 | 37 |
| F | H | I | A | N | 0.239 | 37 |
| C | T | Q | A | G | 0.239 | 37 |
| T | M | V | A | E | 0.240 | 37 |
| A | R | W | A | M | 0.240 | 37 |
| A | L | I | A | N | 0.244 | 36 |
| W | S | A | A | R | 0.245 | 36 |
| W | R | Y | A | P | 0.246 | 36 |
| M | S | Y | A | T | 0.246 | 35 |
| H | N | F | A | G | 0.248 | 35 |
| L | R | I | A | Y | 0.248 | 35 |
| W | R | L | A | N | 0.249 | 35 |
| L | F | L | A | S | 0.249 | 35 |
| S | M | V | A | L | 0.249 | 35 |
| N | S | P | A | G | 0.250 | 34 |
| V | N | G | A | G | 0.251 | 34 |
| A | S | C | A | L | 0.253 | 34 |
| H | Y | N | A | F | 0.263 | 31 |
| Q | G | V | A | K | 0.267 | 30 |
| N | V | R | A | C | 0.268 | 30 |
| V | R | Y | A | L | 0.269 | 29 |
| M | D | N | A | I | 0.270 | 29 |
| E | T | A | A | R | 0.270 | 29 |
| R | S | S | A | G | 0.272 | 29 |
| L | C | I | A | K | 0.275 | 28 |
| S | Y | R | A | D | 0.275 | 28 |
| A | G | G | A | S | 0.276 | 27 |
| V | Y | M | A | R | 0.278 | 27 |
| E | N | F | A | H | 0.281 | 26 |
| A | L | G | A | L | 0.284 | 25 |
| L | L | E | A | C | 0.284 | 25 |
| N | F | S | A | L | 0.285 | 25 |
| T | K | T | A | W | 0.285 | 25 |
| H | S | D | A | C | 0.289 | 24 |
| S | G | R | A | C | 0.290 | 24 |
| E | R | Y | A | H | 0.292 | 23 |
| R | G | A | A | P | 0.293 | 23 |
| R | Y | M | A | V | 0.294 | 23 |
| H | D | T | A | N | 0.295 | 23 |
| T | V | K | A | G | 0.296 | 22 |
| R | R | F | A | E | 0.300 | 21 |
| H | F | V | A | K | 0.301 | 21 |
| D | R | L | A | R | 0.303 | 21 |
| S | N | N | A | A | 0.304 | 20 |
| D | T | P | A | V | 0.311 | 18 |
| D | H | L | A | N | 0.315 | 17 |
| N | I | S | A | T | 0.316 | 17 |
| C | S | I | A | K | 0.317 | 17 |
| Y | R | N | A | N | 0.317 | 17 |
| I | L | K | A | G | 0.317 | 17 |
| G | R | V | A | F | 0.319 | 16 |
| R | S | T | A | D | 0.321 | 16 |
| K | N | T | A | Y | 0.322 | 16 |
| A | P | Y | A | R | 0.326 | 14 |
| K | T | R | A | G | 0.333 | 13 |
| W | A | K | A | R | 0.333 | 13 |
| L | F | K | A | P | 0.334 | 12 |
| S | I | V | A | H | 0.340 | 11 |
| A | N | R | A | R | 0.381 | 0 |

TABLE 5
A513 loop mutants having improved thermostability as evidence
by an at least 5% increase in activity relative to an enzyme
of SEQ ID NO: 1 (comprising ANRAR) following 70° C.
3-minute heat treatment when substrate is casein:

| | | | | | Activity |
|---|---|---|---|---|---|
| 513 | 514 | 515 | 516 | 517 | increase |
| W | R | W | A | Y | 71% |
| N | I | S | A | T | 34% |
| P | R | C | A | E | 34% |
| L | T | G | A | C | 32% |
| F | H | F | A | A | 32% |
| H | C | W | A | F | 24% |
| F | R | Q | A | M | 18% |
| M | Q | V | A | A | 11% |
| A | N | R | A | R | 11% |
| E | L | A | A | L | 9% |
| F | Q | V | A | A | 5% |
| C | C | V | A | C | 5% |
| A | N | R | A | R | n/a |

TABLE 6
A513 loop mutants having improved thermostability as evidence
by an at least 5% increase in activity relative to an enzyme
of SEQ ID NO: 1 (comprising ANRAR) following 72° C.
3-minute heat treatment when substrate is casein.

| | | | | | Average |
|---|---|---|---|---|---|
| 513 | 514 | 515 | 516 | 517 | Activity |
| W | R | W | A | Y | 0.169 |
| N | I | S | A | T | 0.261 |
| P | R | C | A | E | 0.262 |
| L | T | G | A | C | 0.266 |
| F | H | F | A | A | 0.267 |
| H | C | W | A | F | 0.287 |
| F | R | Q | A | M | 0.302 |
| M | Q | V | A | A | 0.319 |
| E | L | A | A | L | 0.325 |
| A | N | R | A | R | 0.347 |

TABLE 7
A513 loop mutants having improved thermostability as
evidence by an at least 5% increase in activity relative
to an enzyme of SEQ ID NO: 1 (comprising ANRAR) following
heat treatment when substrate is casein.

| | | | | | Average |
|---|---|---|---|---|---|
| 513 | 514 | 515 | 516 | 517 | Activity |
| W | R | W | A | Y | 0.168533 |
| Y | N | R | T | R | 0.218033 |
| F | Q | Y | A | G | 0.244467 |
| Y | N | R | T | R | 0.250733 |
| Y | N | R | T | R | 0.2549 |
| S | S | L | A | G | 0.255367 |
| Q | M | Y | A | G | 0.256333 |
| Y | N | R | T | R | 0.2577 |
| N | I | S | A | T | 0.260667 |
| P | R | C | A | E | 0.262133 |
| L | T | G | A | C | 0.265767 |
| F | H | F | A | A | 0.266833 |
| T | L | V | A | G | 0.2687 |
| F | H | I | A | N | 0.269333 |
| Y | Q | V | A | L | 0.2721 |
| H | N | F | A | G | 0.276333 |
| Y | N | R | T | R | 0.277067 |
| D | R | L | A | G | 0.283767 |
| H | C | W | A | F | 0.287 |
| S | T | D | A | G | 0.297033 |
| N | D | L | A | G | 0.3001 |
| F | R | Q | A | M | 0.3016 |
| A | N | R | A | R | 0.3202 |

TABLE 8
A513 loop mutants having improved thermostability as evidence
by an at least 5% increase in activity relative to an enzyme
of SEQ ID NO: 1 (comprising ANRAR) following 72° C.
3-minute heat treatment when substrate is pea protein.

| | | | | | Activity |
|---|---|---|---|---|---|
| 513 | 514 | 515 | 516 | 517 | increase |
| W | R | W | A | Y | 0.169 |
| L | T | G | A | C | 0.266 |
| F | H | F | A | A | 0.267 |
| H | C | W | A | F | 0.287 |
| F | R | Q | A | M | 0.302 |
| T | C | F | A | E | 0.317 |
| M | Q | V | A | A | 0.319 |
| F | Q | V | A | A | n/a |

TABLE 9
A513 loop mutants having improved thermostability as evidence
by an at least 5% increase in activity relative to an enzyme
of SEQ ID NO: 1 (comprising ANRAR) following 70° C.
3-minute heat treatment when substrate is pea protein.

| | | | | | Activity |
|---|---|---|---|---|---|
| 513 | 514 | 515 | 516 | 517 | increase |
| F | R | Q | A | M | 905% |
| F | Q | V | A | A | 477% |
| M | Q | V | A | A | 314% |
| A | L | G | A | L | 312% |
| M | D | N | A | I | 259% |
| L | T | G | A | C | 253% |
| M | C | M | A | K | 246% |
| C | C | V | A | C | 234% |
| A | S | C | A | L | 193% |
| F | H | F | A | A | 171% |
| H | C | W | A | F | 170% |
| N | V | R | A | C | 163% |
| K | A | N | A | C | 152% |
| G | R | V | A | F | 78% |
| W | R | W | A | Y | 68% |
| K | N | T | A | Y | 14% |
| A | N | R | A | R | n/a |

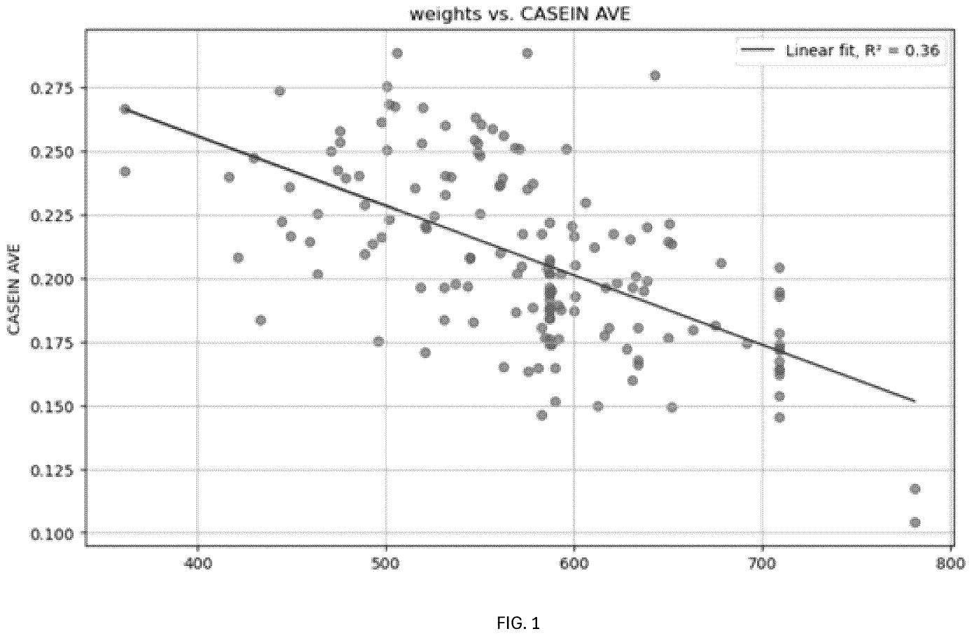

Illustrative data is shown in FIG. 1 to FIG. 4.

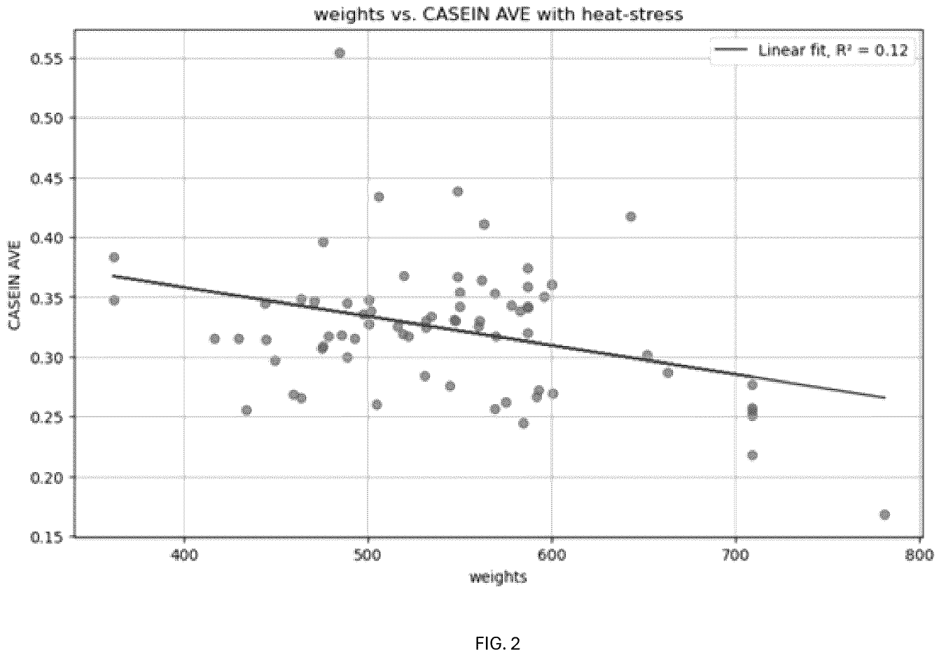

FIG. 1 shows activity changes for various S53 mutants (as individual points) relative to the molecular weight of the amino acids in the A513 loop. The data demonstrates that when the molecular weight for the S53 family protease's 513-loop is greater than 586 kDa, the enzymatic activity of the protease is increased relative to an enzyme including a 513-loop sequence as shown in SEQ ID NO: 1 and which has a molecular weight of about 580 kDa. Similarly, FIG. 2 shows activity changes for various S53 mutants (shown as individual points) relative to the molecular weight of the amino acids in the A513 loop and when the proteases were previously exposed to a heat treatment (e.g., 70° to 72° C. for about three minutes).

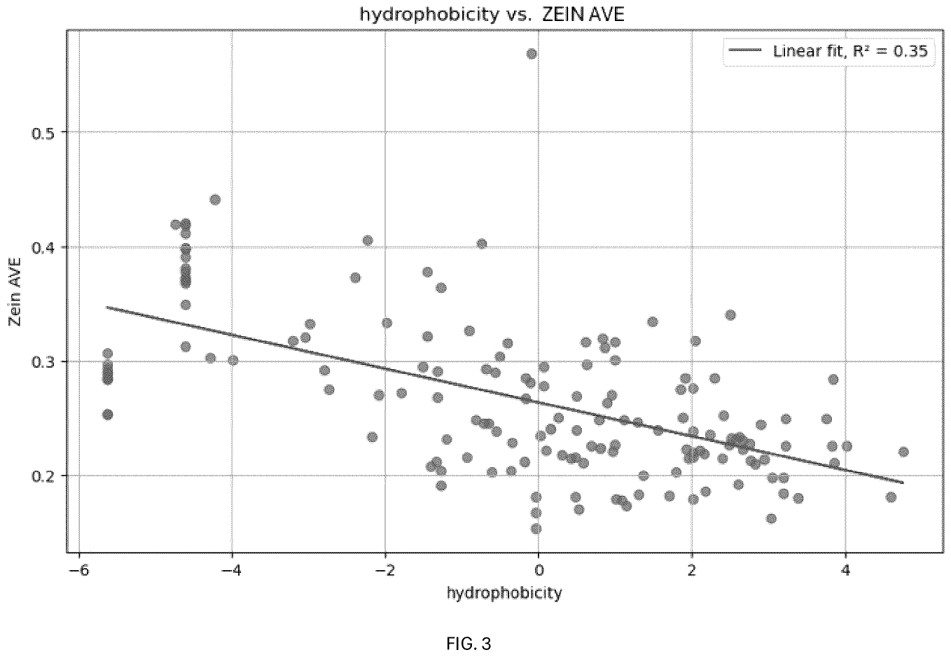

FIG. 3 shows activity changes for various S53 mutants (shown as individual points) relative to the hydrophobicity of the amino acids in the A513 loop. The data demonstrates that when the hydrophobicity for the S53 family protease's 513-loop is greater than −4.6, the enzymatic activity of the protease is increased relative to an enzyme including a 513-loop sequence as shown in SEQ ID NO: 1 and which has a hydrophobicity of about −4.6.

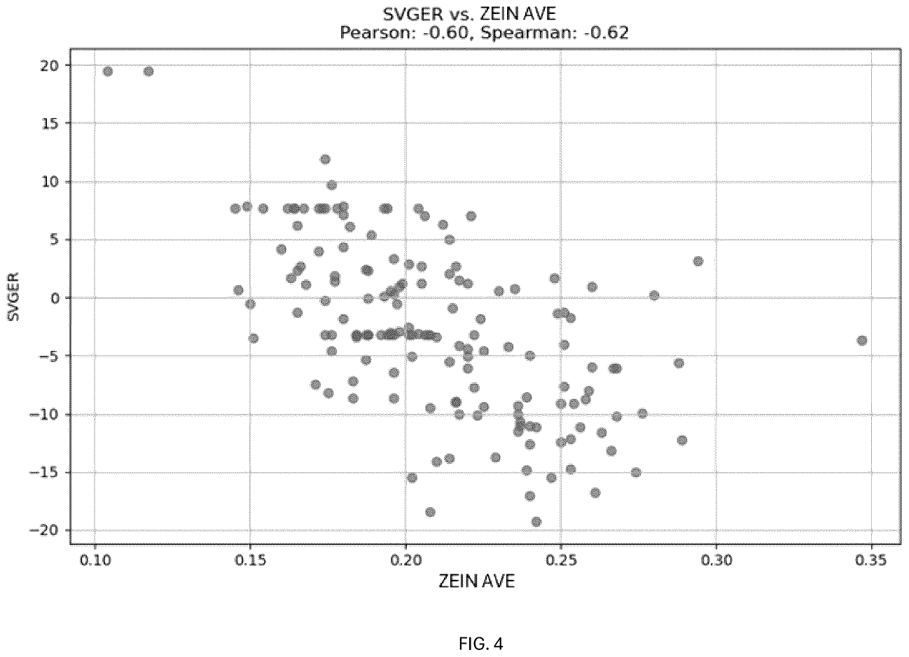

FIG. 4 shows activity changes for various S53 mutants (shown as individual points) relative to the SVGER score of the amino acids in the A513 loop. SVGER is a method for describing the amino acids in a protein. See, e.g., Jianbo Tong, Lingxiao Li, Min Bai, Kangnan Li, “A New Descriptor of Amino Acids-SVGER and its Applications in Peptide QSAR” Molecular Informatics. Volume 36, Issue 5-6 1501023. 14 Oct. 2016. The data demonstrates that when the SVGER score for the S53 family protease's 513-loop is greater than −2.2, the enzymatic activity of the protease is increased relative to an enzyme including a 513-loop sequence as shown in SEQ ID NO: 1 and which has a SVGER score of about −2.2.

### REFERENCES

1. Moughan, R J., Amino acid availability: aspects of chemical analysis and bioassay methodology. Nutrition Research Reviews 2003, 16 (2), 127-141.
2. Elango, R.; Levesque, C.; Ball, R. O.; Pencharz, R B., Available versus digestible amino acids-new stable isotope methods. British Journal of Nutrition 2012, 108 (S2), S306-S314.
3. Misurcova, L., Seaweed digestibility and methods used for digestibility determination. Handbook of Marine Macroalgae: Biotechnology and Applied Phycology 2011, 285-301.
4. Lee, W. T.; Weisell, R.; Albert, J.; Tome, D.; Kurpad, A. V.; Uauy, R., Research Approaches and Methods for Evaluating the Protein Quality of Human Foods Proposed by an FAO Expert Working Group in 2014-The Journal of nutrition 2016, 146 (5), 929-932.
5. Millward, D. J.; Layman, D. K.; Tome, D.; Schaafsma, G., Protein quality assessment: impact of expanding understanding of protein and amino acid needs for optimal health-. The American journal of clinical nutrition 2008, 87 (5), 1576S-1581S.
6. Matthews, D. M.; Adibi, S. A., Peptide absorption. Gastroenterology 1976, 71 (1), 151-161.
7. Sarwar, G.; Peace, R. W.; Bohing, H. G.; Brule, D., Digestibility of protein and amino acids in selected foods as determined by a rat balance method. Plant Foods for Human Nutrition 1989, 39 (1), 23-32.
8. Savoie, L.; Charbonneau, R.; Parent, G., In vitro amino acid digestibility of food proteins as measured by the digestion celltechnique. Plant Foods for Human Nutrition 1989, 39 (1), 93-107.
9. Mandalari, G.; Adel-Patient, K.; Barkholt, V.; Baro, C.; Benneh, L.; Bublin, M.; Gaier, S.; Graser, G.; Ladies, G.; Mierzejewska, D., In vitro digestibility of b-casein and b-lactoglobulin under simulated human gastric and duodenal conditions: a multi-laboratory evaluation. Regulatory Toxicology and Pharmacology 2009, 55 (3), 372-381.
10. Pennings, B.; Boirie, Y.; Senden, J. M.; Gijsen, A. R; Kuipers, H.; van Loon, L. J., Whey protein stimulates postprandial muscle protein accretion more effectively than do casein and casein hydrolysate in older men-. The American journal of clinical nutrition 2011, 93 (5), 997-1005.
11. Koopman, R.; Crombach, N.; Gijsen, A. R; Walrand, S.; Fauquant, I; Kies, A. K.; Lemosquet, S.; Saris, W. H.; Boirie, Y.; van Loon, L. J., Ingestion of a protein hydrolysate is accompanied by an accelerated in vivo digestion and absorption rate when compared with its intact protein-. The American journal of clinical nutrition 2009, 90 (1), 106-115.
12. Oben, J.; Kothari, S. C.; Anderson, M. L., An open label study to determine the effects of an oral proteolytic enzyme system on whey protein concentrate metabolism in healthy males. Journal of the International Society of Sports Nutrition 2008, 5 (1), 10.
13. Astwood, James D., John N. Leach, and Roy L. Fuchs. “Stability of food allergens to digestion in vitro.” Nature biotechnology 14.10 (1996): 1269.
14. Takagi, Kayoko, et aL “Comparative study of in vitro digestibility of food proteins and effect of preheating on the digestion.” Biological and Pharmaceutical Bulletin 26.7 (2003): 969-973.
15. Fu, Tong-Jen, Upasana R. Abbott, and Catherine Hatzos. “Digestibility of food allergens and nonallergenic proteins in simulated gastric fluid and simulated intestinal fluid a comparative study.” Journal of agricultural and food chemistry 50.24 (2002): 7154-7160.
16. Jianbo Tong, Lingxiao Li, Min Bai, Kangnan Li, “A New Descriptor of Amino Acids-SVGER and its Applications in Peptide QSAR” Molecular Informatics. Volume 36, Issue 5-6 1501023. 14 Oct. 2016

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

Further teachings disclosure relevant to the present disclosure are provided in WO2020087017A1 and WO2023147350A1, the contents of each of which is incorporated herein by reference in their entireties.

1. An S53 family protease having an amino acid sequence that is at least 80% identical to SEQ ID NO: 1 and comprises at least one mutation in the amino acid sequence's 513-loop which corresponds to amino acids 513 to 517 of SEQ ID NO: 1, wherein the at least one mutation is located at position 513, 514, 515, and/or 517.

2. The S53 family protease of claim 1, wherein the at least one mutation provides increased thermostability, increased enzymatic activity, and/or increased substrate specificity, relative to an S53 family protease having an amino acid sequence that lacks the at least one mutation located at position 513, 514, 515, and/or 517 with respect to SEQ ID NO: 1.

3. The S53 family protease of claim 1, wherein the S53 family protease comprises at least two mutations in the 513-loop relative to SEQ ID NO: 1.

4. The S53 family protease of claim 3, wherein the at least two mutations are located at positions 513 and 514; at positions 513 and 515; at positions 513 and 517; at positions 514 and 515; at positions 514 and 517; or at positions 515 and 517.

5. The S53 family protease of claim 1, wherein the S53 family protease comprises at least three mutations in the 513-loop relative to SEQ ID NO: 1.

6. The S53 family protease of claim 5, wherein the at least three mutations are located at positions 513, 515, and 517; at positions 513, 514, and 517; at positions 513, 514, and 515; or at positions 514, 515, and 517.

7. The S53 family protease of claim 1, wherein the S53 family protease comprises at least four mutations in the 513-loop relative to SEQ ID NO: 1.

8. The S53 family protease of claim 1, wherein the S53 family protease comprises five mutations in the 513-loop relative to SEQ ID NO: 1 which provide increased enzymatic activity and/or increased substrate specificity.

9. The S53 family protease of claim 1, wherein a mutation at position 513 comprises an A to C substitution, A to D substitution, A to E substitution, A to F substitution, A to G substitution, A to H substitution, A to I substitution, A to K substitution, A to L substitution, A to M substitution, A to N substitution, A to P substitution, A to Q substitution, A to R substitution, A to S substitution, A to T substitution, A to V substitution, A to W substitution, or A to Y substitution.

10. The S53 family protease of claim 1, wherein a mutation at position 514 comprises an N to A substitution, N to C substitution, N to D substitution, N to E substitution, N to F substitution, N to G substitution, N to H substitution, N to I substitution, N to K substitution, N to L substitution, N to M substitution, N to Q substitution, N to R substitution, N to S substitution, N to T substitution, N to V substitution, N to W substitution, or N to Y substitution.

11. The S53 family protease of claim 1, wherein a mutation at position 515 comprises an R to A substitution, R to C substitution, R to D substitution, R to E substitution, R to F substitution, R to G substitution, R to I substitution, R to K substitution, R to L substitution, R to M substitution, R to N substitution, R to P substitution, R to Q substitution, R to S substitution, R to T substitution, R to V substitution, R to W substitution, or R to Y substitution.

12. The S53 family protease of claim 1, wherein a mutation at position 517 comprises an R to A substitution, R to C substitution, R to D substitution, R to E substitution, R to F substitution, R to G substitution, R to H substitution, R to I substitution, R to K substitution, R to L substitution, R to M substitution, R to N substitution, R to P substitution, R to Q substitution, R to S substitution, R to T substitution, R to V substitution, R to W substitution, or R to Y substitution.

13. The S53 family protease of claim 1, wherein the at least one mutation in the amino acid sequence's 513-loop is as shown in one of Table 1 to Table 9.

14. The S53 family protease of claim 1, further comprising one or more additional mutations at position 13, 15, 18, 23, 35, 38, 40, 51, 52, 55, 64, 70, 73, 91, 96, 101, 121, 123, 143, 145, 147, 155, 174, 175, 181, 188, 198, 218, 228, 229, 236, 240, 243, 253, 261, 265, 268, 273, 280, 283, 286, 290, 292, 296, 297, 300, 303, 308, 312, 314, 319, 323, 324, 325, 326, 327, 328, 329, 330, 332, 341, 353, 359, 360, 371, 377, 385, 387, 390, 391, 392, 398, 404, 411, 417, 421, 422, 432, 433, 434, 441, 446, 448, 452, 459, 469, 474, 486, 499, 500, 501, 509, 513, 514, 515, 516, 517, 537, and/or 550 relative to SEQ ID NO: 1.

15. The S53 family protease of claim 14, wherein the one or more additional mutations comprises substitutions selected from K13A, E15A, A18Q, R23A, T35A, Q38E, I40M, G51A, D52G, E55A, L64I, V70E, E73K, I91L, E96D, T101A, V121I, Q123R, V143L, D145E, R147T, L155M, L174M, R175Q, E181G, S188A, T228A, S236S, S240P, T253S, N261S, I283F, N297D, V303I, H308L, I312V, A325T, P326S, S328A, A387G, E391A, R392Q, S404A, S417A, R421H, G433S, V441L, T459A, P486A, P499A, E500D, R517Q, I537V, and/or A550P relative to SEQ ID NO: 1.

16. The S53 family protease of claim 14, wherein the one or more additional mutations comprises substitutions selected from A325K; A325L; P326F; P326L; P326W; S328Q;
S328H; E391V; E391L; E391I; E391M; S417W; V441W; T459W; T459R; P486Q; and/or R517G relative to SEQ ID NO: 1.

17. The S53 family protease of claim 1, further comprising one or more substrate selectivity mutations wherein the one or more substrate selectivity mutations increase protease substrate selectivity relative to a protease comprising an amino acid sequence as shown in SEQ ID NO: 1 and wherein the one or more additional mutations is at position 198, 218, 229, 243, 265, 268, 273, 280, 286, 290, 292, 296, 300, 314, 319, 323, 324, 325, 326, 327, 328, 329, 330, 332, 341, 353, 359, 360, 371, 377, 385, 390, 391, 398, 411, 417, 422, 432, 433, 434, 441, 446, 448, 452, 459, 469, 474, 486, 501, 509, 513, 514, 515, 516, and/or 517 relative to SEQ ID NO: 1.

18. The S53 family protease of claim 17, wherein the one or more substrate selectivity mutations comprises substitutions selected from D198E, I218L, S229D, Q243G, G265A, E268M, E268T, E268C, E268H, E268Q, V273I, G280R, Y286K, N290T, N290S, D292F, D292Y, L296T, T300S, S314P, G319Q, S323R, W324K, A325K, A325L, P326F, P326W, P326L, A327Y, A327D, S328H, S328Q, I329L, A330F, M332I, M332L, A341R, S353E, E359V, E359L, Q360C, A371S, A377G, A385V, A385L, I390W, E391V, E391M, E391L, E391I, D398S, R411Q, R411M, R411E, R411D, S417W, A422V, A432G, G433G, S434I, S434R, S434T, S434V, V441W, D446S, A448N, E452Y, E452W, E452F, T459R, T459W, A469V, A474V, P486Q, V501C, N509H, A513T, A513D, A513Y, N514G, R515L, R515E, R515I, A516T, A516S, and/or R517G relative to SEQ ID NO: 1.

19-92. (canceled)

93. A food product comprising an S53 family protease having an amino acid sequence that is at least 80% identical to SEQ ID NO: 1 and comprises at least one mutation in the amino acid sequence's 513-loop which corresponds to amino acids 513 to 517 of SEQ ID NO: 1 and a protein obtained from or naturally present in Adzuki beans, Almonds, Amaranth, Asparagus, Baby Lima, Barley, Beef, Black beans, Black eyed peas, Broccoli, Buckwheat, Cannellini beans, Cashews, Chia seeds, Chicken, Chickpea, Chlorella, Corn, Cowpea, Cranberry beans, Crowder pea, Egg, e.g., chicken egg, Fava beans, Field Peas, Flounder, Great Northern Beans, Green beans, Hemp protein powder, Kamut, Kidney beans, Lady cream peas, Lentil, Lupine Beans, Masoor Dal (Indian Red lentils), Milk or other milk products, e.g., cheese and yoghurt, Mung beans, Navy pea, Pea, Peanut, Pigeon Peas, Pink beans, Pinto beans, Pistachios, Pork, Quinoa, Royal Canin, Rye berries, Salmon, Soy, Spirulina, Sunflower seeds, Turkey, White beans, and Yellow split peas.

94-118. (canceled)

119. A method for improving the digestion of proteins in a food product by a subject, the method comprising ingesting with the food product, a food supplement comprising an S53 family protease having an amino acid sequence that is at least 80% identical to SEQ ID NO: 1 and comprises at least one mutation in the amino acid sequence's 513-loop which corresponds to amino acids 513 to 517 of SEQ ID NO: 1.

120-144. (canceled)


SEQUENCE LISTING (11 sequences)

SEQ ID NO: 1 - protein, 552 aa, Bacillus sp.
MSDMEKPWKEEEKREVLAGHARRQAPQAVDKGPVTGDQRISVTVVLRRQRGDELEAHVERQAALAPHARVHLEREAFAASHGASLDDFAEIRKFAEAHGLTLDRAHVAAGTAVLSGPVDAVNQAFGVELRHFDHPDGSYRSYVGDVRVPASIAPLIEAVLGLDTRPVARPHFRLRRRAEGEFEARSQSAAPTAYTPLDVAQAYQFPEGLDGQGQCIAIIELGGGYDETSLAQYFASLGVSAPQVVSVSVDGATNQPTGDPNGPDGEVELDIEVAGALAPGAKIAVYFAPNTDAGFLNAITTAVHDPTHKPSIVSISWGGPEDSWAPASIAAMNRAFLDAAALGVTVLAAAGDSGSTDGEQDGLYHVDFPAASPYVLACGGTRLVASAGRIERETVWNDGPDGGSTGGGVSRIFPLPSWQERANVPPSANPGAGSGRGVPDVAGNADPATGYEVVIDGETTVIGGTSAVAPLFAALVARINQKLGKPVGYLNPTLYQLPPEVFHDITEGNNDIANRARIYQAGPGWDPCTGLGSPIGIRLLQALLPSASQAQP

SEQ ID NO: 2 - protein, 5 aa, synthetic construct
ANRAR

SEQ ID NO: 3 - protein, 5 aa, synthetic construct
ANRAQ

SEQ ID NO: 4 - protein, 552 aa, synthetic construct
MSDMEKPWKEEEKREVLAGHARRQAPQAVDKGPVTGDQRISVTVVLRRQRGDELEAHVERQAALAPHARVHLEREAFAASHGASLDDFAEIRKFAEAHGLTLDRAHVAAGTAVLSGPVDAVNQAFGVELRHFDHPDGSYRSYVGDVRVPASIAPLIEAVLGLDTRPVARPHFRLRRRAEGEFEARSQSAAPTAYTPLDVAQAYQFPEGLDGQGQCIAIIELGGGYDETSLAQYFASLGVSAPQVVSVSVDGATNQPTGDPNGPDGEVELDIEVAGALAPGAKIAVYFAPNTDAGFLNAITTAVHDPTHKPSIVSISWGGPEDSWAPASIAAMNRAFLDAAALGVTVLAAAGDSGSTDGEQDGLYHVDFPAASPYVLACGGTRLVASAGRIERETVWNDGPDGGSTGGGVSRIFPLPSWQERANVPPSANPGAGSGRGVPDVAGNADPATGYEVVIDGETTVIGGTSAVAPLFAALVARINQKLGKPVGYLNPTLYQLPPEVFHDITEGNNDIANRAQIYQAGPGWDPCTGLGSPIGIRLLQALLPSASQAQP

SEQ ID NO: 5 - protein, 552 aa, synthetic construct
MSDMEKPWKEEEKREVLAGHARRQAPQAVDKGPVTGDQRISVTVVLRRQRGDELEAHVERQAALAPHARVHLEREAFAASHGASLDDFAEIRKFAEAHGLTLDRAHVAAGTAVLSGPVDAVNQAFGVELRHFDHPDGSYRSYVGDVRVPASIAPLIEAVFGLDTRPVARPHFRLRRRAEGEFEARSQSAAPTAYTPLDVAQAYQFPEGLDGQGQCIAIIELGGGYDETSLAQYFASLGVSAPQVVSVSVDGATNQPTGDPNGPDGEVELDIEVAGALAPGAKIAVYFAPNTDAGFLNAITTAVHDPTHKPSIVSISWGGPEDSWAPASIAAMNRAFLDAAALGVTVLAAAGDSGSTDGEQDGLYHVDFPAASPYVLACGGTRLVASAGRIERETVWNDGPDGGSTGGGVSRIFPLPSWQERANVPPSANPGAGSGRGVPDVAGNADPATGYEVVIDGETTVIGGTSAVAPLFAALVARINQKLGKPVGYLNPTLYQLPPEVFHDITEGNNDIANRARIYQAGPGWDPCTGLGSPIGIRLLQALLPSASQAQP

SEQ ID NO: 6 - DNA, 68 bp, synthetic construct
  source 1..68: mol_type = other DNA
cgctccgcaggttgttagcgttagcgtgnnknnknnknnkaatcagccgacaggtgatccgaatggtc

SEQ ID NO: 7 - DNA, 26 bp, synthetic construct
  source 1..26: mol_type = other DNA
gaggcagctgatacagtgtcggattc

SEQ ID NO: 8 - DNA, 26 bp, synthetic construct
  source 1..26: mol_type = other DNA
gaatccgacactgtatcagctgcctc

SEQ ID NO: 9 - DNA, 27 bp, synthetic construct
  source 1..27: mol_type = other DNA
cacgctaacgctaacaacctgcggagc

SEQ ID NO: 10 - DNA, 69 bp, synthetic construct
  source 1..69: mol_type = other DNA
ccgggtgcaaaaattgcagtttattttgcaccgnnknnknnkgccggttttctgaatgcaattaccacc

SEQ ID NO: 11 - DNA, 33 bp, synthetic construct
  source 1..33: mol_type = other DNA
cggtgcaaaataaactgcaatttttgcacccgg